SEQUENCE LISTING (32 sequences)

SEQ ID NO: 1 - protein, 120 aa, synthetic construct
  REGION 1..120: note = VH
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSS

SEQ ID NO: 2 - protein, 107 aa, synthetic construct
  REGION 1..107: note = VL
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIK

SEQ ID NO: 3 - protein, 22 aa, synthetic construct
  REGION 1..22: note = Signal/leader peptide (GM-CSF)
MLLLVTSLLLCELPHPAFLLIP

SEQ ID NO: 4 - protein, 28 aa, synthetic construct
  REGION 1..28: note = CD28tm
MFWVLVVVGGVLACYSLLVTVAFIIFWV

SEQ ID NO: 5 - protein, 38 aa, synthetic construct
  REGION 1..38: note = CD28 linker
IEVMYPPPYLDNEKSNGTIIHVKGKHLCPSPLFPGPSK

SEQ ID NO: 6 - protein, 66 aa, synthetic construct
  REGION 1..66: note = CD28 linker and transmembrane domain
IEVMYPPPYLDNEKSNGTIIHVKGKHLCPSPLFPGPSKPFWVLVVVGGVLACYSLLVTVAFIIFWV

SEQ ID NO: 7 - protein, 41 aa, synthetic construct
  REGION 1..41: note = CD28 cytoplasmic
RSKRSRLLHSDYMNMTPRRPGPTRKHYQPYAPPRDFAAYRS

SEQ ID NO: 8 - protein, 42 aa, synthetic construct
  REGION 1..42: note = 4-1BB signaling domain
KRGRKKLLYIFKQPFMRPVQTTQEEDGCSCRFPEEEEGGCEL

SEQ ID NO: 9 - protein, 112 aa, synthetic construct
  REGION 1..112: note = CD3-zeta chain signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 21 aa, synthetic construct
  REGION 1..21: note = T2A
GGGEGRGSLLTCGDVEENPGP

SEQ ID NO: 11 - protein, 335 aa, synthetic construct
  REGION 1..335: note = EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 12 - protein, 12 aa, synthetic construct
  REGION 1..12: note = S (small) spacer
ESKYGPPCPPCP

SEQ ID NO: 13 - protein, 119 aa, synthetic construct
  REGION 1..119: note = M (medium) spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 229 aa, synthetic construct
  REGION 1..229: note = L (long) spacer
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 1045 aa, synthetic construct
  REGION 1..1045: note = VHVL "S" Construct
MLLLVTSLLLCELPHPAFLLIPQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGGGGSGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 16 - protein, 1152 aa, synthetic construct
  REGION 1..1152: note = VHVL "M" Construct
MLLLVTSLLLCELPHPAFLLIPQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGGGGSGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 1262 aa, synthetic construct
  REGION 1..1262: note = VHVL "L" Construct
MLLLVTSLLLCELPHPAFLLIPQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGGGGSGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 18 - protein, 1045 aa, synthetic construct
  REGION 1..1045: note = VLVH "S" Construct
MLLLVTSLLLCELPHPAFLLIPDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGGGGSGGGGSGGGGSQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 19 - protein, 1152 aa, synthetic construct
  REGION 1..1152: note = VLVH "M" Construct
MLLLVTSLLLCELPHPAFLLIPDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGGGGSGGGGSGGGGSQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 20 - protein, 1262 aa, synthetic construct
  REGION 1..1262: note = VLVH "L" Construct
MLLLVTSLLLCELPHPAFLLIPDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGGGGSGGGGSGGGGSQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKNDGGGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 187 aa, synthetic construct
  REGION 1..187: note = DHFRdm
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 22 - protein, 22 aa, synthetic construct
  REGION 1..22: note = P2A
GSGATNFSLLKQAGDVEENPGP

SEQ ID NO: 23 - protein, 242 aa, synthetic construct
  REGION 1..242: note = scFv (VHVL)
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGGGGSGGGGSGGGGSDIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIK

SEQ ID NO: 24 - protein, 242 aa, synthetic construct
  REGION 1..242: note = scFv (VLVH)
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGGGGSGGGGSGGGGSQVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSS

SEQ ID NO: 25 - protein, 231 aa, synthetic construct
  REGION 1..231: note = EF1 promoter
AGGCTCCGGTGCCCGTCAGTGGGCAGAGCGCACATCGCCCACAGTCCCCGAGAAGTTGGGGGGAGGGGTCGGCAATTGAACCGGTGCCTAGAGAAGGTGGCGCGGGGTAAACTGGGAAAGTGATGTCGTGTACTGGCTCCGCCTTTTTCCCGAGGGTGGGGGAGAACCGTATATAAGTGCAGTAGTCGCCGTGAACGTTCTTTTTCGCAACGGGTTTGCCGCCAGAACACA

SEQ ID NO: 26 - protein, 15 aa, synthetic construct
  REGION 1..15: note = Linker
GGGGSGGGGSGGGGS

SEQ ID NO: 27 - protein, 5 aa, synthetic construct
  REGION 1..5: note = HVR-H1
DTFFQ

SEQ ID NO: 28 - protein, 16 aa, synthetic construct
  REGION 1..16: note = HVR-H1
RIDPANGFTKYAQKFQ

SEQ ID NO: 29 - protein, 11 aa, synthetic construct
  REGION 1..11: note = HVR-H1
WDTYGAAWFAY

SEQ ID NO: 30 - protein, 11 aa, synthetic construct
  REGION 1..11: note = HVR-L1
RASGNIHNYLS

SEQ ID NO: 31 - protein, 7 aa, synthetic construct
  REGION 1..7: note = HVR-L2
SAKTLAD

SEQ ID NO: 32 - protein, 9 aa, synthetic construct
  REGION 1..9: note = HVR-L3
QHFWSSIYT

(19) United States
(12) Patent Application Publication
(10) Pub. No.: US 2026/0184759 A1
(43) Pub. Date: Jul. 2, 2026

(54) CHIMERIC ANTIGEN RECEPTORS SPECIFIC FOR CONJUGATED BIOTIN

(71) Applicant: Seattle Children's Hospital (dba Seattle Children's Research Institute), Seattle, WA (US)

(72) Inventors: Clinton M. Heinze, Seattle, WA (US); Michael C. Jensen, Seattle, WA (US)

(21) Appl. No.: 19/130,507

(22) PCT Filed: Nov. 17, 2023
(86) PCT No.: PCT/US2023/080257

§ 371 Date: May 15, 2025

Related U.S. Application Data
(60) Provisional application No. 63/384,560, filed on Nov. 21, 2022.

(51) Int. Cl. C07K 14/725; C07K 16/44

(52) U.S. Cl. CPC C07K 14/7051; C07K 16/44; C07K 2317/622; C07K 2317/92

(57) ABSTRACT

Some embodiments provided herein include methods and compositions comprising chimeric antigen receptors (CARs) which specifically bind biotin conjugated to a molecule (conjugated biotin). Some embodiments include therapies for inhibiting, ameliorating, or treating a disorder, such as cancer, in a subject. Some such embodiments include administration of a biotinylated composition, such as conjugated biotin, in combination with the CAR to the subject. In some such embodiments, an immune cell, such as a T cell comprises the CAR.

### CROSS-REFERENCE TO RELATED APPLICATIONS

This application claims priority to U.S. Prov. App. No. 63/384,560 filed Nov. 21, 2022 entitled “CHIMERIC ANTIGEN RECEPTORS SPECIFIC FOR CONJUGATED BIOTIN” which is expressly incorporated by reference herein in its entirety.

### REFERENCE TO SEQUENCE LISTING

The present application is being filed along with a Sequence Listing in electronic format. The Sequence Listing is provided as a file entitled SCRI360WOSEQLIST.xml, created Nov. 13, 2023, which is approximately 49,743 bytes in size. The information in the electronic format of the Sequence Listing is incorporated herein by reference in its entirety.

### FIELD OF THE INVENTION

Some embodiments provided herein include methods and compositions comprising chimeric antigen receptors (CARs), which specifically bind biotin conjugated to a molecule (conjugated biotin). Some embodiments include therapies for inhibiting, ameliorating, or treating a disorder, such as cancer, in a subject. Some such embodiments include administration of a biotinylated composition, such as the conjugated biotin, in combination with the CAR to the subject. In some such embodiments, an immune cell, such as a T cell comprises the CAR.

### BACKGROUND OF THE INVENTION

Chimeric antigen receptors (CARs), are also referred to as artificial T-cell receptors, chimeric T-cell receptors, chimeric immunoreceptors and chimeric receptors, can graft a desired specificity onto an immune effector cell. For example, these receptors can be used to graft the specificity of a monoclonal antibody onto a T-cell.

Adoptive transfer of human T lymphocytes that are engineered by gene transfer to express chimeric antigen receptors (chimeric receptors) specific for surface molecules expressed on tumor cells have the potential to effectively treat advanced malignancies and cancer. Chimeric receptors are synthetic receptors that can include an extracellular ligand binding domain, which can be an antibody or a binding portion thereof and most commonly, it is a single chain variable fragment of a monoclonal antibody (scFv) linked to intracellular signaling components, most commonly CD3ζ alone, or it can be combined with one or more co-stimulatory domains, such as, for example, 4-1BB. Much of the research in the design of chimeric receptors has focused on defining scFvs and other ligand binding elements that target malignant cells without causing serious toxicity to normal cells and tissues.

As this field is still in its infancy, there remains a need for methods for determining the elements of chimeric receptor design that are important for therapeutic activity and approaches to identify host cell populations suitable for genetic modification and adoptive cell transfer so as to generate targeted regenerative cell therapeutics, which exhibit enhanced survival and efficacy in vivo.

### SUMMARY OF THE INVENTION

Some embodiments of the methods and compositions disclosed herein include an isolated polynucleotide encoding a chimeric antigen receptor (CAR) capable of specifically binding to biotin conjugated to a molecule (conjugated biotin), wherein the CAR comprises: (i) an scFv domain configured to bind to conjugated biotin; (ii) a spacer domain; (iii) a transmembrane domain; and (iv) an intracellular signaling domain. In some embodiments, the scFv domain selectively binds to conjugated biotin as compared to biotin not conjugated to a molecule (free biotin). In some embodiments, the scFv domain comprises a VH domain and a VL domain.

In some embodiments, the VH domain comprises: (a) a heavy chain hyper variable region 1 (HVR-H1) comprising the amino acid sequence of SEQ ID NO: 27 comprising 0-3 conservative substitutions thereof; (b) a heavy chain hyper variable region 2 (HVR-H2) comprising the amino acid sequence of SEQ ID NO: 28 comprising 0-3 conservative substitutions thereof; and/or (c) a heavy chain hyper variable region 3 (HVR-H3) comprising the amino acid sequence of SEQ ID NO: 29 comprising 0-3 conservative substitutions thereof. In some embodiments, the VH domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1.

In some embodiments, the VL domain comprises: (a) a light chain hyper variable region 1 (HVR-L1) comprising the amino acid sequence of SEQ ID NO: 30 comprising 0-3 conservative substitutions thereof; (b) a light chain hyper variable region 2 (HVR-L2) comprising the amino acid sequence of SEQ ID NO: 31 comprising 0-3 conservative substitutions thereof; and/or (c) a light chain hyper variable region 3 (HVR-L3) comprising the amino acid sequence of SEQ ID NO: 32 comprising 0-3 conservative substitutions thereof. In some embodiments, the VL domain comprises an amino acid sequence having at least or at least about 80%, 81%, 82%, 83%, 84%, 85%, 86%, 87%, 88%, 89%, 90%, 91%, 92%/a, 93%, 94%, 95%, 96%, 97%, 98%, 99%, or 100% sequence identity to the amino acid sequence of SEQ ID NO: 2.

In some embodiments, the VH4 domain is located in the polypeptide N-terminal of the VL domain. In some embodiments, the VL domain is located in the polypeptide N-terminal of the VH domain. In some embodiments, the VH domain and the VL domain are joined via a linker domain. In some embodiments, the linker domain comprises or consists of the amino acid sequence of SEQ ID NO:23. In some embodiments, the scFv domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 23 or 24.

In some embodiments, the intracellular signaling domain comprises a 4-1BB domain, a CD3-zeta domain, and/or a CD28tm domain. In some embodiments, the 4-1BB domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 8. In some embodiments, the CD3-zeta domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the CD28tm domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO; 4.

In some embodiments, the spacer domain comprises an IgG4 hinge domain, and IgG4 hinge-CH3 domain, or an IgG4 hinge -CH2-CH3 domain. In some embodiments, the spacer domain has a length: (i) in a range greater than or equal to 1 and less than or equal to 12 consecutive amino acids, or a length of 12 residues or about 12 residues; (ii) in a range from greater than or equal to 13 and less than or equal to 119 consecutive amino acids, or a length of 119 residues or about 119 residues; or (iii) in a range greater than or equal to 120 and less than or equal to 229 consecutive amino acids, or a length of 229 residues or about 229 residues. In some embodiments, the spacer domain comprises an amino acid sequence having at least or at least about 80%, 81%, 82%, 83%, 84%, 85%, 86%, 87%, 88%, 89%, 90%, 91%, 92%, 93%, 94/o, 95%, 96%, 97%, 98%, 99%, or 100% sequence identity to the amino acid sequence of any one of SEQ ID NOs: 12, 13 or 14. In some embodiments, the CAR comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 15-20.

In some embodiments, the CAR further comprises a signal peptide.

In some embodiments, the polynucleotide further comprises a nucleic acid encoding a selectable marker. In some embodiments, the selectable marker is selected from a truncated EGFR (EGFRt) polypeptide or a truncated Her2 (Her2t)polypeptide. In some embodiments, the selectable marker comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 11.

In some embodiments, the polynucleotide further comprises a ribosomal skip sequence. In some embodiments, the ribosomal skip sequence is selected from T2A, P2A, E2A, or F2A. In some embodiments, the ribosomal skip sequence is located in the polynucleotide between a nucleic acid encoding the CAR and the nucleic acid encoding the selectable marker,

In some embodiments, the conjugated biotin comprises or consists of biotin conjugated to the molecule via a linker. In some embodiments, the molecule is selected from fluorescein, a fluorescein derivative, folic acid, folate, a folate derivative, dinitrophenol (DNP), a phospholipid ether (PLE), polyethylene glycol (PEG), a lipid, a protein, a Bite, or an aptamer. In some embodiments, the linker comprises a PEG or a polypeptide.

In some embodiments, the conjugated biotin is selected from the group consisting of: FA-biotin. FSL-biotin or any other biotinylated lipid such as biotin-PLE, fluorescently-labeled biotin, biotin-folate, a biotinylated small molecule, biotin-PEG3-Cy5, a biotinylated protein, biotinylated peptide, biotinylated antibody, or biotinylated aptamer.

In some embodiments, the conjugated biotin comprises biotin conjugated to folate via a polypeptide linker. In some embodiments, the conjugated biotin has a mass in a range from about 500 Da to about 1000 Da or 500 Da to 1000 Da. In some embodiments, the conjugated biotin has a mass of about 700 Da or 700 Da.

Some embodiments of the methods and compositions disclosed herein include a vector comprising the polynucleotide of any one of the embodiments of the present disclosure. In some embodiments, the vector is a viral vector. In some embodiments, the viral vector is a lentivirus, an adeno-associated virus (AAV).

Some embodiments of the methods and compositions disclosed herein include a CAR encoded by the polynucleotide of any one of the embodiments of the present disclosure.

Some embodiments of the methods and compositions disclosed herein include a composition comprising any one of the CARs provided herein bound to the conjugated biotin. In some embodiments, the conjugated biotin comprises or consists of biotin conjugated to the molecule via a linker. In some embodiments, the molecule is selected from fluorescein, a fluorescein derivative, folic acid, folate, a folate derivative, dinitrophenol (DNP), a phospholipid ether (PLE), polyethylene glycol (PEG), a lipid, a protein, a Bite, or an aptamer. In some embodiments, the linker comprises a PEG or a polypeptide. In some embodiments, the conjugated biotin is selected from the group consisting of. FA-biotin, FSL-biotin or any other biotinylated lipid such as biotin-PLE, fluorescently-labeled biotin, biotin-folate, a biotinylated small molecule, biotin-PEG3-Cy5, a biotinylated protein, biotinylated peptide, biotinylated antibody, or biotinylated aptamer. In some embodiments, the conjugated biotin comprises biotin conjugated to folate via a polypeptide linker. In some embodiments, the conjugated biotin has a mass in a range from about 500 Da to about 1000 Da or 500 Da to 1000 Da. In some embodiments, the conjugated biotin has a mass of about 700 Da or 700 Da. In some embodiments, the spacer domain of the CAR has a length in a range greater than or equal to 120 and less than or equal to 229 consecutive amino acids. In some embodiments, the spacer domain of the CAR has a length of 229 residues or about 229 residues. In some embodiments, the conjugated biotin has a mass in a range from about 500 Da to about 1000 Da or 500 Da to 1000 Da; and the spacer domain of the CAR has a length in a range greater than or equal to 120 and less than or equal to 229 consecutive amino acids. In some embodiments, the composition also includes a cell comprising the CAR. In some embodiments the cell is an immune cell, such as a T cell.

Some embodiments of the methods and compositions disclosed herein include a cell comprising the polynucleotide of any one of the embodiments of the present disclosure, or the CAR of any one of the embodiments of the present disclosure. In some embodiments, the cell is an immune cell. In some embodiments, the cell is selected from a T-cell, a natural killer cell, a T cell precursor, and a hematopoietic stem cell. In some embodiments, the cell is a CD4+ T-cell or a CD8+ T-cell. In some embodiments, the cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell. In some embodiments, the cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell. In some embodiments, the cell is mammalian. In some embodiments, the cell is human. In some embodiments, the cell is in vitro or ex vivo. In some embodiments, the cell is in vivo.

Some embodiments of the methods and compositions disclosed herein include a pharmaceutical composition comprising the cell of any one of the embodiments of the present disclosure, and a pharmaceutically acceptable excipient.

Some embodiments of the methods and compositions disclosed herein include a method of preparing the cell of any one of the embodiments of the present disclosure, comprising transducing or transfecting a cell with the polynucleotide of any one of the embodiments of the present disclosure, to obtain a transduced cell. In some embodiments, the method further comprises selecting for the transduced cell, optionally, wherein the selecting comprises selecting for a transduction marker; optionally, wherein the transduction marker comprises a truncated EGFR (EGFRt) polypeptide or a truncated Her2 (Her2t)polypeptide. In some embodiments, the method further comprises contacting the transduced cell with an anti-CD3 antibody, an anti-CD28 antibody, and/or antigen binding fragment thereof.

Some embodiments of the methods and compositions disclosed herein include a method for treating, inhibiting or ameliorating a disorder in a subject, comprising administering the cell of any one of the embodiments of the present disclosure to the subject. In some embodiments, the method further comprises administering the conjugated biotin to the subject. In some embodiments, the conjugated biotin is capable of specifically binding to a cell comprising the disorder; optionally, wherein the conjugated biotin comprises a folate, a folate derivative, a phospholipid ether (PLE), an antibody capable of binding to a cancer antigen or an antigen-binding fragment thereof. In some embodiments, the conjugated biotin comprises biotin conjugated to folate via a polypeptide linker. In some embodiments, the conjugated biotin has a mass in a range from about 500 Da to about 1000 Da or 500 Da to 1000 Da; and the spacer domain of the CAR has a length in a range greater than or equal to 120 and less than or equal to 229 consecutive amino acids. In some embodiments, the cell is autologous to the subject. In some embodiments, the cell is allogeneic to the subject. In some embodiments, the cell is modified to reduce a likelihood of an allogeneic response in the subject; optionally, wherein the cell lacks expression of an MHC class I protein, an MHC class II protein, an HLA protein, or an HLA-E protein. In some embodiments, the cell is administered prior to administration of the conjugated biotin. In some embodiments, the cell is administered at least 1 day prior to administration of the conjugated biotin. In some embodiments, the cell is administered subsequent to administration of the conjugated biotin. In some embodiments, the cell is administered to the subject at least 1 day subsequent to administration of the conjugated biotin. In some embodiments, the subject is administered more than a single dose of the cell. In some embodiments, the subject is administered more than a single dose of the conjugated biotin. In some embodiments, the subject is administered no more than a single dose of the cell. In some embodiments, the subject is administered no more than a single dose of the conjugated biotin. In some embodiments, the disorder is a cancer or a viral infection; optionally, wherein the cancer is a solid tumor or a leukemia. In some embodiments, the cancer is selected from a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer. In some embodiments, the solid tumor is capable of specifically binding the conjugated biotin. In some embodiments, the solid tumor is selected from breast cancer, rhabdomyosarcoma, Ewing sarcoma, neuroblastoma, alveolar soft-part sarcoma, osteosarcoma, synovial sarcoma, soft tissue sarcoma, glioblastoma, medulloblastoma, or diffuse intrinsic pontine glioma (DIPG, midline granuloma). In some embodiments, the subject is mammalian. In some embodiments, the subject is human.

Some embodiments of the methods and compositions disclosed herein include a system for use as a medicament such as a medicament for use in treating, inhibiting, or ameliorating a disorder, such as a cancer or a viral disease, in a subject, comprising: (i) the polynucleotide of any one of the embodiments of the present disclosure, the CAR of any one of the embodiments of the present disclosure, or the cell of any one of the embodiments of the present disclosure; and (ii) the conjugated biotin. In some embodiments, the conjugated biotin is capable of specifically binding to a cell comprising the disorder. In some embodiments; the cell is a cancer cell. In some embodiments, the conjugated biotin is capable of specifically binding to a cancer antigen. In some embodiments, the conjugated biotin comprises a folate, a folate derivative, a phospholipid ether (PLE), an antibody capable of binding to a cancer antigen or an antigen-binding fragment thereof. In some embodiments, the conjugated biotin is selected from the group consisting of: FA-biotin, FSL-biotin or any other biotinylated lipid such as biotin-PLE, fluorescently-labeled biotin, biotin-folate, a biotinylated small molecule, biotin-PEG3-Cy5, a biotinylated protein, biotinylated peptide, biotinylated antibody, or biotinylated aptamer. In some embodiments, the conjugated biotin comprises biotin conjugated to folate via a polypeptide linker. In some embodiments, the conjugated biotin has a mass in a range from about 500 Da to about 1000 Da or 500 Da to 1000 Da. In some embodiments, the conjugated biotin has a mass of about 700 Da or 700 Da. In some embodiments, the conjugated biotin comprises biotin conjugated to folate via a polypeptide linker. In some embodiments, the conjugated biotin has a mass in a range from about 500 Da to about 1000 Da or 500 Da to 1000 Da; and the spacer domain of the CAR has a length in a range greater than or equal to 120 and less than or equal to 229 consecutive amino acids. In some embodiments, the disorder comprises a cancer, or a viral infection; optionally, wherein the cancer comprises a solid tumor or a leukemia; optionally, wherein the cancer is a breast cancer, a rhabdomyosarcoma, a Ewing sarcoma, a neuroblastoma, an alveolar soft-part sarcoma, an osteosarcoma, a synovial sarcoma, a soft tissue sarcoma, a glioblastoma, a medulloblastoma, or a diffuse intrinsic pontine glioma (DIPG, midline granuloma).

Some embodiments of the methods and compositions disclosed herein include use of the polynucleotide of any one of the embodiments of the present disclosure, the CAR of any one of the embodiments of the present disclosure, or the cell of any one of the embodiments of the present disclosure, as a medicament such as in the preparation of a medicament or use thereof for treating, inhibiting, or ameliorating a disorder, such as a viral disease or a cancer in a subject. In some embodiments, the medicament and/or use is in combination with the conjugated biotin. In some embodiments, the conjugated biotin is capable of specifically binding to a cell comprising the disorder. In some embodiments, the cell is a cancer cell. In some embodiments, the conjugated biotin is capable of specifically binding to a cancer antigen.

### BRIEF DESCRIPTION OF THE DRAWINGS

FIG. 1 depicts a non-limiting example of a VH sequence (SEQ ID NO: 1) and VL sequence (SEQ ID NO: 2).

FIG. 2A depicts an example of a schematic of a plasmid encoding conjugated-biotin-targeting CARs.

FIG. 2B depicts a schematic of repulsion of free biotin from a biotin receptor (left panel); and binding of conjugated biotin to a biotin receptor.

FIG. 3A and FIG. 3B each depict flow cytometry analysis of mixed CD4/CD8 cultures transduced with various polynucleotides encoding anti-conjugated biotin CARs. Cells were stained with an anti-EGFR antibody to assess transduction marker expression. The percentage of cells with positive EGFRt and MFI positive labeling are listed next to histograms for VHVL (FIG. 3A) and VLVH (FIG. 3B) CARs. Staining was conducted on S1D9.

FIG. 4A and FIG. 4B each depict flow cytometry analysis of mixed CD4/CD8 cultures transduced with various polynucleotides encoding anti-conjugated biotin CARs. Cells were stained with an anti-Fc antibody to directly measure CAR expression. The percentage of cells with Fc and MFI positive labeling are listed next to histograms for VHVL (FIG. 4A) and VLVH (FIG. 4B) CARs. Staining was conducted on S1D9.

FIG. 5 depicts a CD3z western blot analysis of anti-biotin CAR expression in SIR1D15 cells. CAR sizes were verified with benchmark CARs with long (anti-FITC), medium (anti-B7H3), and short (anti-CD19) spacers.

FIG. 6 depicts soluble biotin conjugated to a Cy5 label via a PEG linker (biotin-PEG-Cy5) with a molecular weight about 1136.39 Da.

FIG. 7A depicts biotin conjugated to folate via a PEG linker (Biotin-PEG2k-folate; intermediate 2 of Example 4).

FIG. 7B depicts a structure of non-conjugated biotin.

FIG. 8A and FIG. 8B each depict flow cytometry analysis of CARs incubated with 440 nM molecule 1 (soluble antigen) for 30 minutes at room temperature. Cy5+ cells and MFI are listed next to histograms for VHVL (FIG. 8A) and VLVH (FIG. 8B) CARs. The assay was performed on S1R1D12.

FIG. 9 depicts a graph for an anti-biotin CAR binding curve. VLVH anti-conjugated biotin CAR T cells were incubated with titrated concentrations of biotin-PEG-Cy5. A best-fit line was calculated in GraphPad Prism to model the data and the binding affinity was calculated to be 0.553 nM.

FIG. 10 depicts a function-spacer-lipid (FSL)-biotin (FSL-biotin; intermediate 1 of Example 4) having a molecular weight of about 2079.28 kDa. In some embodiments, FSL-biotin labels cell surfaces by incorporating into cell membranes. In some embodiments, FSL-biotin serves as an intermediate between target cells and CAR T cells.

FIG. 11A and FIG. 11B each depict bar graphs of MFIs from flow cytometry analysis of VHVL and VLVH CARs concurrently incubated with 440 nM (50 ng) biotin-PEG-Cy5 (soluble antigen) and 0 nM (no competition), 0.2 nM, 1 nM, 5 nM, or 20 nM of competition molecules unconjugated biotin (FIG. 11A) or biotin-folate (FIG. 11B) for 30 minutes at room temperature. The experiment was performed on S1R1D12 cells.

FIG. 12A and FIG. 12B each depict bar graphs of activation marker CD107a expressed in CD8+ anti-biotin VHVL (FIG. 12A) and VLVH (FIG. 12B) CAR T cells co-incubated with the indicated target cells for 4 hours at 37° C. Activation markers were upregulated when stimulated with both intermediate-labelled cells but not unlabeled cells. The assay was performed on S1R1D11.

FIG. 13A and FIG. 13B each depict bar graphs of activation marker nur77 expressed in CD8+ anti-biotin VHVL (FIG. 13A) and VLVH (FIG. 13B) CAR T cells co-incubated with the indicated target cells for 4 hours at 37° C. Activation markers were upregulated when stimulated with both intermediate-labelled cells but not unlabeled cells. The assay was performed on S1R1D11.

FIG. 14A and FIG. 14B each depict bar graphs of activation marker 41BB expressed in CD8+ anti-biotin VHVL (FIG. 14A) and VLVH (FIG. 14B) CAR T cells co-incubated with the indicated target cells for 4 hours at 37° C. Activation markers were upregulated when stimulated with both intermediate-labelled cells but not unlabeled cells. The assay was performed on S1R1D11.

FIG. 15 depicts a series of pie charts of intracellular cytokines produced in CD4+ anti-biotin CAR T cells co-incubated with the indicated target cells for 4 hours at 37° C. The assay was performed on S1R1D11.

FIG. 16A and FIG. 16B each depict VHVL (FIG. 16A) and VLVH (FIG. 16B) graphs of specific lysis of K562 parental target cells incubated for 4 hours at 37° C. at various ratios with mixed CD4/8 anti-biotin CAR T cells. Specific lysis was measured by chromium released from chromium-loaded target cells (chromium loaded overnight at 37° C.). The assay was performed on S1R1D14.

FIG. 17A and FIG. 17B each depict VHVL (FIG. 17A) and VLVH (FIG. 17B) graphs of specific lysis of K562 parental target cells labeled with FSL-biotin (intermediate 1 of Example 4) and incubated for 4 hours at 37° C. at various ratios with mixed CD4/8 anti-biotin CAR T cells. Specific lysis was measured by chromium released from chromium-loaded target cells (chromium loaded overnight at 37° C.). The assay was performed on S1R1D14.

FIG. 18A and FIG. 18B each depict VHVL (FIG. 18A) and VLVH (FIG. 18B) graphs of specific lysis of K562 OKT3 target cells and incubated for 4 hours at 37° C. at various ratios with mixed CD4/8 anti-biotin CAR T cells. Specific lysis was measured by chromium released from chromium-loaded target cells (chromium loaded overnight at 37° C.). The assay was performed on S1R1D14.

FIG. 19A and FIG. 19B each depict VHVL (FIG. 19A) and VLVH (FIG. 19B) graphs of specific lysis of MDA-MB-231 target cells incubated for 4 hours at 37° C. at various ratios with mixed CD4/8 anti-biotin CAR T cells. Specific lysis was measured by chromium released from chromium-loaded target cells (chromium loaded overnight at 37° C.). The assay was performed on S1R1D14.

FIG. 20A and FIG. 20B each depict VHVL (FIG. 20A) and VLVH (FIG. 20B) graphs of specific lysis of MDA-MB-231 target cells labeled with biotin-folate (intermediate 2 of Example 4) and incubated for 4 hours at 37° C. at various ratios with mixed CD4/8 anti-biotin CAR T cells. Specific lysis was measured by chromium released from chromium-loaded target cells (chromium loaded overnight at 37° C.). The assay was performed on S1R1D14.

FIG. 21A and FIG. 21B each depict IFN released into media from mixed CD4/8 anti-biotin VHVL (FIG. 21A) and VLVH (FIG. 21B) CAR T cells incubated with target cells for 24 hours at 37° C. The assay was performed on S1R1D13.

FIG. 22A and FIG. 22B each depict TNF released into media from mixed CD4/8 anti-biotin VHVL (FIG. 22A) and VLVH (FIG. 22B) CAR T cells incubated with target cells for 24 hours at 37° C. The assay was performed on S1R1D13.

FIG. 23A and FIG. 23B each depict IL2 cytokines released into media from mixed CD4/8 anti-biotin VHVL (FIG. 23A) and VLVH (FIG. 23B) CAR T cells incubated with target cells for 24 hours at 37° C. The assay was performed on S1R1D13.

FIG. 24 depicts flow cytometry analysis to confirm intermediate 1 labeling of K562 P target cells. K562 P (“unstained”) or FSL-biotin-labelled cells (“target+intermediate”) were stained with a streptavidin-APC conjugate.

FIG. 25 depicts flow cytometry analysis to confirm intermediate 2 labeling of MDA-MB-231 target cells. MDA-MB-231 (“unstained”) or FSL-biotin-labelled cells (“target+intermediate”) were stained with a streptavidin-APC conjugate.

FIG. 26 depicts FACS analyses for CD4/8 distribution including the CD4/8 distribution for VHVL “L,” or long spacer cells, VLVH L cells, VHVL “M”, or medium spacer cells, VLVH M cells, VHVL “S”, or short spacer cells, VLVH S cells, and mock cells. The assay was performed on S1R1D15.

FIG. 27A depicts flow cytometry analysis for unlabeled K562 CD19 cells or K562 CD19 cells labeled with a biotinylated anti-CD19 antibody. Biotin presentation on each group is assessed by staining with streptavidin-APC.

FIG. 27B depicts pie charts for the distribution of triple positive, IFN+/IL2+, IFN+/TNF+, INF+, IL2+/TNF+, IL2+, TNF+, and triple negative cytokine production in CD4+ anti-biotin CAR T cells co-incubated with the indicated target cells for 4 hours at 37° C. The assay was performed on S1R1D8.

FIG. 28 depicts a series of pie charts of activation markers expressed on CD8+ anti-biotin CAR T cells co-incubated with the indicated target cells for 4 hours at 37° C. The assay was performed on S1R1D8.

FIG. 29A depicts flow cytometry analysis to confirm intermediate labeling of K562 P target cells. K562 P or FSL-biotin-labelled cells were stained with a streptavidin-APC conjugate.

FIG. 29B depicts a series of pie charts of intracellular cytokines produced in CD4+ anti-biotin CAR T cells co-incubated with the indicated target cells and indicated concentrations of unconjugated biotin for 4 hours at 37° C. The assay was performed on S1R1D10.

FIG. 30A depicts flow cytometry analysis to confirm intermediate labeling of MDA-MB-231 target cells. MDA-MB-231 or biotin-folate-labelled MDA-MB-231 cells were stained with a streptavidin-APC conjugate.

FIG. 30B depicts a series of pie charts of intracellular cytokines produced in CD4+ anti-biotin CAR T cells co-incubated with the indicated target cells and indicated concentrations of unconjugated biotin for 4 hours at 37° C. The assay was performed on S1R1D10.

FIG. 31 depicts a series of pie charts of activation/exhaustion markers expressed on CD8+ anti-biotin CAR T cells co-incubated with the indicated target cells and indicated concentrations of unconjugated biotin for 4 hours at 37° C. The assay was performed on S1R1D10.

FIG. 32 depicts a series of pie charts of activation/exhaustion markers expressed on CD8+ anti-biotin CAR T cells co-incubated with the indicated target cells and indicated concentrations of unconjugated biotin for 4 hours at 37° C. The assay was performed on S1R1D11.

FIG. 33A depicts the percent specific lysis of K562 OKT3 target cells incubated with mixed CD4/8 VHVL anti-conjugated biotin CAR T cells for 4 hours at 37° C. Specific lysis of target cells with VHVL (long), VHVL (medium), or VHVL (short) was measured by chromium released from chromium-loaded target cells (chromium loaded overnight at 37° C.). The ratio of effector cells to target cells was either 30:1, 10:1, 3:1, or 1:1.

FIG. 33B depicts the percent specific lysis of K562 OKT3 target cells incubated with mixed CD4/8 VLVH anti-conjugated biotin CAR T cells for 4 hours at 37° C. Specific lysis of target cells with VLVH (long), VLVH (medium), or VLVH (short) was measured by chromium released from chromium-loaded target cells (chromium loaded overnight at 37° C.). The ratio of effector cells to target cells was either 30:1, 10:1, 3:1, or 1:1.

FIG. 34A depicts the percent specific lysis of K562 CD19 target cells incubated with mixed CD4/8 VHVL anti-conjugated biotin CAR T cells for 4 hours at 37° C. Specific lysis of target cells with VHVL (long), VHVL (medium), or VHVL (short) was measured by chromium released from chromium-loaded target cells (chromium loaded overnight at 37° C.). The ratio of effector cells to target cells was either 30:1, 10:1, 3:1, or 1:1.

FIG. 34B depicts the percent specific lysis of K562 CD19 target cells incubated with mixed CD4/8 VLVH anti-conjugated biotin CAR T cells for 4 hours at 37° C. Specific lysis of target cells with VLVH (long), VLVH (medium), or VLVH (short) was measured by chromium released from chromium-loaded target cells (chromium loaded overnight at 37° C.). The ratio of effector cells to target cells was either 30:1, 10:1, 3:1, or 1:1.

FIG. 35A depicts the percent specific lysis of K562 CD19 target cells pre-labelled with a biotinylated anti-CD19 antibody and incubated with mixed CD4/8 VHVL anti-conjugated biotin CAR T cells for 4 hours at 37° C. Specific lysis of target cells with VHVL (long), VHVL (medium), or VHVL (short) was measured by chromium released from chromium-loaded target cells (chromium loaded overnight at 37° C.). The ratio of effector cells to target cells was either 30:1, 10:1, 3:1, or 1:1.

FIG. 35B depicts the percent specific lysis of K562 CD19 target cells pre-labelled with a biotinylated anti-CD19 antibody and incubated with mixed CD4/8 VLVH anti-conjugated biotin CAR T cells for 4 hours at 37° C. Specific lysis of target cells with VLVH (long), VLVH (medium), or VLVH (short) was measured by chromium released from chromium-loaded target cells (chromium loaded overnight at 37° C.). The ratio of effector cells to target cells was either 30:1, 10:1, 3:1, or 1:1.

FIG. 36A depicts the percent specific lysis of K562 parental target cells incubated with mixed CD4/8 VHVL anti-conjugated biotin CAR T cells for 4 hours at 37° C. Specific lysis of target cells with VHVL (long), VHVL (medium), or VHVL (short) was measured by chromium released from chromium-loaded target cells (chromium loaded overnight at 37° C.). The ratio of effector cells to target cells was either 30:1, 10:1, 3:1, or 1:1.

FIG. 36B depicts the percent specific lysis of K562 parental target cells incubated with mixed CD4/8 VLVH anti-conjugated biotin CAR T cells for 4 hours at 37° C. Specific lysis of target cells with VLVH (long), VLVH (medium), or VLVH (short) was measured by chromium released from chromium-loaded target cells (chromium loaded overnight at 37° C.). The ratio of effector cells to target cells was either 30:1, 10:1, 3:1, or 1:1.

FIG. 37A depicts the percent specific lysis of K562 parental target cells pre-labelled with a biotinylated anti-CD19 antibody and incubated with mixed CD4/8 VHVL anti-conjugated biotin CAR T cells for 4 hours at 37° C. Specific lysis of target cells with VLVH (long), VLVH (medium), or VLVH (short) was measured by chromium released from chromium-loaded target cells (chromium loaded overnight at 37° C.). The ratio of effector cells to target cells was either 30:1, 10:1, 3:1, or 1:1.

FIG. 37B depicts the percent specific lysis of K562 parental target cells pre-labelled with a biotinylated anti-CD19 antibody and incubated with mixed CD4/8 VLVH anti-conjugated biotin CAR T cells for 4 hours at 37° C. Specific lysis of target cells with VHVL (long), VHVL (medium), or VHVL (short) was measured by chromium released from chromium-loaded target cells (chromium loaded overnight at 37° C.). The ratio of effector cells to target cells was either 30:1, 10:1, 3:1, or 1:1.

FIG. 38A depicts illustrative schematics of the domains, as well as their corresponding SEQ ID NOS, for the VHVL “S”, VHVL “M”, and VHVL “L” constructs.

FIG. 38B depicts illustrative schematics of the domains, as well as their corresponding SEQ ID NOS, for the VLVH “S”, VLVH “M”, and VLVH “L” constructs.

FIG. 39 depicts a time course for cell production and assays. On day 9 of the original cell production, cells were characterized, and the antigen binding/competition assays were conducted. Five million cells from the original cell production were then expanded using the rapid cell expansion protocol (REP). Remaining cells were frozen down and used for in vivo experiments. Functional assays were performed after day 11 of REP, and a Western blot was conducted at the end of the study. Cells were cultured in methotrexate (MTX) from day 4 during each expansion.

FIG. 40A and FIG. 40B each depict a schematic of a biotin intermediate adaptor used in functional assays. Each intermediates has a conjugated biotin epitope, a flexible linker domain, and a tumor-targeting domain. FIG. 40A depicts a FSL-biotin intermediate/adapter which was used to label K562 Parental (K562 P) targets via integration into the cell membrane. FIG. 40B depicts a Biotin-PEG2k-folate intermediate/adapter which was used to label folate receptor-expressing MDA-MB-231 targets.

FIG. 41 depicts a graph from a bilayer interferometry (BLI) binding study to characterize biotin-PEG2k-folate affinity for FOLR1 protein. Biotin-PEG2k-folate was immobilized on streptavidin biosensor tips and exposed to descending doses of soluble FOLR1 protein (25 nM to 0.78125 nM). Data was fit and a dynamic Kd of 556+1-3.56 pM was calculated.

FIG. 42A depicts a graph for total integrated intensity (RCU×μm2/image) of mCherry signal from intermediate 2-labelled, mCherry-expressing MDA-MB-231 cells. VLVH anti-conjugated biotin CAR T cells were incubated with target cells at a 1:1 E:T ratio and imaged every other hour in an IncuCyte cell imaging system. CAR T cells were re-challenged with labeled target cells at 72 hours and 144 hours (indicated by dashed lines). n=3 technical replicates per condition. Data is presented as mean values+/−SEM. Data is representative of two donors. Intensities over 300,000 were excluded for plot clarity.

FIG. 42B depicts a graph a schematic diagram of the timelines for in vivo experiments with biotin CAR T cells. Five million MDA-MB-231 cells were subcutaneously engrafted in NSG mice. On day 0, 10E6 T cells were injected IV. On days −1, 2, 9, 16, and 23, 500 nmol/kg of drug was injected IV. Tumors were measured and mice were imaged with an IVIS twice per week until the conclusion of the study.

FIG. 42C depicts graphs for individual tumor measurements in each treatment group and shows survival of all CAR T cell-treated mice beyond the completion of treatment.

FIG. 42D depicts a Kaplan-Meier survival curve showing extended survival of animals (n=5) treated with anti-biotin CAR T cells+biotin-PEG2k-folate compared with mock+biotin-PEG2k-folate. A Mantel-Cox log-rank test was performed with a Bonferroni correction for multiple comparisons. “*” denotes significance of p<0.01667 for three comparisons. Exact p-values are p=0.009 for the short spacer, p=0.009 for the medium spacer, and p=0.0047 for the long spacer. No significant difference in animal survival between spacer groups was observed.

FIG. 42E depicts individual bioluminescence images from the spacer comparison in vivo study. Mice were treated with anti-biotin CAR T cells or mock cells on day 0. 500 nmol/kg biotin-folate or FITC-folate (EC17) were administered on days −1, 2, 9, 16, and 23. Each CAR was able to slow tumor growth but not eliminate any tumors. Mouse 2 from the anti-biotin(short)+biotin-folate group survived to day 51.

FIG. 42F depicts graphs for measurements of changes in mouse bodyweights. No significant drops in mouse body weight were observed in any group throughout the study (n=5).

FIG. 43 depicts graphs for a comparison of anti-tumor function enabled by biotin-folate conjugates with increasing molecular weights. MDA-MB-231 cells were labeled with 2 kDa, 5 kDa, or 10 kDa biotin-folate. Target cells were then incubated with VLVH short spacer anti-biotin CAR or mock T cells for 24 hours. Supernatant was harvested and cytokine concentration was quantified with a Meso Scale Delivery (MSD) kit.

FIG. 44A depicts a bar graph showing the percentage of CD107a+CD8+ anti-conjugated biotin CAR T cells following a 4-hour 1:1 E:T incubation with intermediate-labeled or unlabeled MDA-MB-231 target cells. Data are representative of trends in Nur77 and 4-1BB upregulation under the same conditions and same experimental groups. Graphs are representative of two donors.

FIG. 44B depicts a graph for a Meso Scale Discovery Assay quantifying IFNγ released into media by mixed CD4/CD8 anti-conjugated biotin CAR T cells after a 24-hour 2:1 E:T co-culture with target cells. Data are representative of trends in IL-2 and TNFαreleased under the same conditions and same experimental groups with two donors. n=3 technical replicates per condition. Data is presented as mean values+/−SD.

FIG. 44C depicts a graph for total integrated intensity (RCU×μm2/image) of mCherry signal from intermediate-labelled, mCherry-expressing MDA-MB-231 cells. Long or short spacer VLVH anti-conjugated biotin CAR T cells were incubated with target cells at a 1:1 E:T ratio and imaged every other hour in an IncuCyte cell imaging system. CAR T cells were re-challenged with labeled target cells at 72 hours and 144 hours (indicated with dashed lines). n=3 technical replicates per condition. Data is presented as mean values+/−SEM. Data is representative of two donors. Intensities over 600,000 were excluded for plot clarity.

FIG. 44D depicts a graph for caliper measurements of individual tumors.

FIG. 44E depicts a Kaplan-Meier survival curve showing animal survival (n=3) after treatment with anti-biotin CAR T cells+our two biotin folate compounds compared with one another and negative controls. A Mantel-Cox log-rank test was performed with a Bonferroni correction for multiple comparisons. After Bonferroni correction was performed, no significant difference in animal survival between groups was observed.

FIG. 45A depicts bar graphs showing the percentage of 41BB+ (upper panel), or Nur77+(lower panel) CD8+ anti-conjugated biotin CAR T cells following a 4-hour 1:1 E:T incubation with intermediate-labeled MDA-MB-231 target cells.

FIG. 45B depicts pie charts showing the makeup of intracellular cytokines produced in CAR T cells incubated 1:1 E:T with targets for 4 hours.

FIG. 46A depicts a graph for a cytokine release assay using a Meso Scale Discovery kit for IL2 released into media by mixed CD4/CD8 anti-conjugated biotin CAR T cells after a 24-hour 2:1 E:T co-culture with target cells. n=3 technical replicates per condition. Data is presented as mean values+/−SD.

FIG. 46B depicts a graph for a cytokine release assay using a Meso Scale Discovery kit for TNFα released into media by mixed CD4/CD8 anti-conjugated biotin CAR T cells after a 24-hour 2:1 E:T co-culture with target cells. n=3 technical replicates per condition. Data is presented as mean values+/−SD.

FIG. 47A depicts IVIS images of tumor burden over time.

FIG. 47B depicts caliper measurements of individual tumors.

FIG. 47C depicts survival of treated mice over time. A Mantel-Cox log-rank test was performed with a Bonferroni correction for multiple comparisons. “*” denotes significance of p<0.0125 for four comparisons, n=10 mice for all Biotin (L) groups and n=5 for mock and FL CAR groups.

### DETAILED DESCRIPTION

Some embodiments provided herein include methods and compositions comprising chimeric antigen receptors (CARs) which specifically bind biotin conjugated to a molecule (conjugated biotin). Some embodiments include therapies for inhibiting, ameliorating, or treating a disorder, such as cancer, in a subject. Some such embodiments include administration of a biotinylated composition in combination with the CAR to the subject.

CAR T cell therapy has demonstrated potent efficacy against hematological malignancies and holds great potential for the treatment of other cancers. However, conventional, monovalent CAR T cells can have high relapse rates and limited success in antigenically heterogenous solid tumors. The lack of versatility provided by standard CARs has led to the development of universal CARs, a class of highly modular and controllable synthetic receptors capable of multivalent antigen targeting via bifunctional intermediate adaptor molecules. Continuing the evolution of universal CAR systems toward increasingly biocompatible designs, provided herein is a new panel of CARs that selectively targeted any conjugated derivative of biotin. This selectivity for conjugated, but not unmodified, biotin was used with well-tolerated molecules to target a multiplicity of tumor antigens while avoiding therapeutic interference by dietary biotin. The versatility of the system to work with different intermediate adaptor molecules to mount a response against different tumors is described herein. Also described herein, is use of the system in a model in vivo application, where CAR T cell durability and robustness to eliminate aggressive solid tumors was demonstrated.

CAR-based therapy for hematologic malignancies has changed clinical practice and deepened the understanding of human tumor immunology. Fusion-positive (FP)-RMS is more aggressive and most in need of CAR-T based therapy due to its extremely low mutational burden. Mesenchymal cancers (sarcomas) differ from epithelial cancers (carcinomas) in that no EMT (epithelial to mesenchymal transition) is required for metastasis, and the tumor microenvironment differs.

Anti-fluorescein CARs are currently used in hapten-specific CARs orthogonal to endogenous tissue. As described herein, the introduction of CARs specific for conjugated biotin, such as biotin conjugated to a molecule, expands the repertoire of hapten-specific CARs. Some embodiments provided herein include use of two orthogonal CAR T cell therapies for refined therapies and new cellular circuits. For example, certain embodiments include therapies utilizing both an anti-fluorescein CAR and an anti-conjugated biotin CAR.

Some embodiments include an AND-gated anti-tumor function. In some such embodiments, both biotin and fluorescein are present on a tumor cell surface. Here, both biotin and fluorescein are conjugated to moities, such as, small molecules, lipids, proteins, peptides, or aptamers, that bind to the tumor for a partial anti-fluorescein CAR (e.g. zeta only) and partial anti-conj. biotin CAR (e.g. 4-1BB only) recognition and activation. In such embodiments, the anti-fluorescein CAR (zeta) and anti-conjugated biotin CAR (4-1BB) when simultaneously stimulated give the same response as a second generation CAR.

Some embodiments include an IF-THEN-gating of anti-tumor function. In some such embodiments, a biotinylated molecule binds to a small subset of a tumor or to a characteristic marker of the tumor microenvironment. Binding of a constitutively expressed anti-conjugated biotin CAR to the biotinylated molecule drives production of an inducible anti-fluorescein CAR within the same cell. The cells now expressing the anti-fluorescein CAR can then bind a more broadly targeting chemical (e.g. FL-PLE) for a direct anti-tumor response. This restricts the CAR T cells to only be active in the tumor microenvironment.

Some embodiments include an oscillating therapy using both anti-conj. biotin and anti-fluorescein CAR T cells and both biotin and fluorescein bispecific molecules (e.g. biotin-PLE and FL-PLE). In such embodiments, active and rest periods are controlled for both classes of CAR T cells via delivery of appropriate targeting molecules. This oscillating therapy includes longer persistence of CAR T cells by preventing terminally exhausted cells while continuously attacking the tumor.

Disclosed herein are chimeric antigen receptor (CAR) T cells specific to conjugated biotin. Also disclosed herein are therapies, which use the CAR T cells specific to conjugated biotin. In some embodiments, the conjugated biotin-directed CAR T cell therapy targets biotinylated tumor-targeting molecules, such as lipids, chemicals, proteins, peptides, and aptamers for the treatment of a cancer. Also disclosed herein is a highly regulatable CAR T cell therapy that works orthogonal to endogenous tissue via an otherwise immunologically inert small molecule.

In some embodiments, the anti-conjugated (anti-conj.) biotin CAR T cell differs from previous anti-biotin CAR T cells due to its explicit ability to only bind conjugated biotin (as opposed to free biotin, for example biotin not conjugated to another molecule), thereby mitigating the impact of biotin present in cultures and in the body on CAR T cell function. Some such embodiments include CAR T cells which target conjugated biotin and which are not functionally impacted by the presence of unconjugated biotin. Some embodiments described herein include retention of CAR function in vivo where unconjugated biotin is present, thereby providing orthogonality to endogenous tissue.

Certain aspects disclosed in the following may be useful with embodiments of the methods and compositions provided herein: U.S. Pat. Nos. 9,765,153; 11,311,576; 11,649,288; US 2020/0354477; Weichert, J. P., et al. (2014) Science Translational Medicine, 6(240), 240ra75-240ra75; Van der Luit, A. H., et al. (2007) Molecular Cancer Therapeutics, 6(8), 2337-2345; Ma, J. S. Y., et al. (2016) PNAS USA, 113(4), E450-E458; Lee, Y. G., et al. (2019) Cancer Research, 79(2), 387-396; and Lu, Y. et al. (2019) Frontiers in Oncology. See www.frontiersin.org/articles/10.3389/fonc.2019.00151/full; Lohmueller, J. J. et al., OncoImmunology, 7:1, DOI: 10.1080/2162402X.2017.1368604, which are each incorporated by reference herein in its entirety.

### Certain Definitions

Unless defined otherwise, all technical and scientific terms used herein have the same meaning as commonly understood by one of ordinary skill in the art to which the invention pertains.

As used herein, “a” or “an” may mean one or more than one.

The term “comprising” as used herein is synonymous with “including,” “containing,” or “characterized by,” and is inclusive or open-ended and does not exclude additional, unrecited elements or method steps.

“About” as used herein when referring to a measurable value is meant to encompass variations of ±20% or +10%, more preferably ±5%, even more preferably ±1%, and still more preferably ±0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally occurring nucleotides (such as DNA or RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid encoding the chimeric antigen receptor specific for conjugated biotin.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or CARs. These receptors can be used to graft the specificity of a monoclonal antibody or binding fragment thereof onto a cell, preferably a T-cell, with transfer of their coding sequence facilitated by any methodology that is well established in the field, such as electroporation, nucleofection, or with viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. CARs are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

As used herein, an “antibody” has its plain and ordinary meaning when read in light of the specification, and includes, for example, a large Y-shape protein produced by plasma cells that is used by the immune system to identify and neutralize foreign objects such as bacteria and viruses. The antibody protein can comprise four polypeptide chains: two identical heavy chains and two identical light chains connected by disulfide bonds. Each chain is composed of structural domains called immunoglobulin domains. These domains can contain or contain about 70, 80, 90, 100, 110, 120, 130, 140, or 150 amino acids or any number of amino acids in between in a range defined by any two of these values and are classified into different categories according to their size and function. In some embodiments, the ligand binding domain comprises a CDR, an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand. In some embodiments, the antibody fragment or binding portion thereof present on a CAR is specific for conjugated biotin. In some embodiments, the ligand binding domain is an antibody fragment or a binding portion thereof, such as a VH domain, or a single chain variable fragment (scFv) containing VH and VL domains. In some embodiments, the antibody fragment or binding portion thereof present on a CAR comprises one or more domains from a humanized antibody, or binding portion thereof.

As used herein, “complementarity-determining regions” or “CDR” includes certain portions of the variable region in an antibody or antigen binding fragment thereof, which forms the binding and specificity of various specific antibodies to their particular antigen. Variability is not uniformly distributed throughout an antibody variable region, and may be focused concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a pi-folded configuration, joined by three CDRs which form a linking loop, and in some cases can form a partially β-folded structure. The CDRs in each chain are closely adjacent to the others by the FR regions and form an antigen-binding site of the antibody with the CDRs of the other chain. The constant regions are not directly involved in the binding of the antibody to the antigen, but they exhibit different effects or functions, for example, involving in antibody-dependent cytotoxicity of the antibodies.

As used herein, “hypervariable region” or “HVR”, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Example hypervariable loops occur at amino acid residues 26-32 (L1), 50-52 (L2), 91-96 (L3), 26-32 (H1), 53-55 (H2), and 96-101 (H3). (Chothia, C. and Lesk, A. M., J. Mol. Biol. 196 (1987) 901-917) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat, E. A., et al., Sequences of Proteins of Immunological Interest, 5th ed. Public Health Service, National Institutes of Health, Bethesda, Md. (1991), NIH Publication 91-3242.) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Example a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro, J. C. and Fransson, J., Front. Biosci. 13 (2008) 1619-1633). Unless otherwise indicated, HVR residues and other residues in the variable domain (e.g., FR residues) are numbered herein according to Kabat et al. U.S. Pat. No. 9,765,153 is incorporated by reference herein in its entirety.

As used herein, a “single chain variable fragment” or “scFv” has its plain and ordinary meaning when read in light of the specification, and includes, for example, a fusion protein of the variable regions of the heavy (VH) and light chains (VL) of immunoglobulins, connected with a short linker peptide of ten to 25 amino acids or about 25 amino acids. In some embodiments, a CAR is provided, wherein the CAR comprises a ScFv, a VH domain, and/or a VL domain, specific for conjugated biotin.

The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a VH domain, a receptor ligand or mutants thereof, peptide, and/or polypeptide affinity molecule or binding partner. Without being limiting, a ligand binding domain can be a specific protein domain or an epitope on a protein that is specific for a ligand or ligands.

Some embodiments include a spacer. In some embodiments, the peptide spacer is 15 amino acids or less but not less than 1 or 2 amino acids. In some embodiments, the spacer is a polypeptide chain. In some aspects, the polypeptide chain may range in length, such as from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78,. 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208 209, 210, 211, 212, 213, 214, 215, 216, 217,218, 219, 220, 221, 222, 223, 224, 225, 226,227, 228, 229, 230,231, 232, 233, 234, 235, 236, 237, 238, 239 or 240 amino acids or a length within a range defined by any two of the aforementioned lengths. A spacer can comprise any 20 amino acids, for example, in any order to create a desirable length of polypeptide chain in a CAR, which includes the amino acids arginine, histidine, lysine, aspartic acid, glutamic acid, serine, threonine, asparagine, glutamine, cysteine, glycine, proline, alanine, valine, isoleucine, methionine, phenylalanine, tyrosine or tryptophan. A spacer sequence can be a linker between the ligand binding domain and the transmembrane domain of the CAR. In some embodiments, the chimeric antigen receptor further comprises a sequence encoding a spacer. In some embodiments, the spacer comprises a sequence with a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78,. 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117,118, 119, 120, 121, 122, 123, 124, 125, 126,127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214,215, 216, 217, 218, 219, 220, 221,222, 223, 224, 225, 226, 227, 228, 229, 230, 231, 232, 233, 234, 235, 236, 237, 238, 239 or 240 amino acids or a length within a range defined by any two of the aforementioned lengths. In some embodiments, the spacer resides between the ligand binding domain and the transmembrane region of the CAR. In some embodiments, the spacer resides between the ligand binding domain of the CAR and the transmembrane region of the CAR.

A spacer may also be customized, selected, or optimized for a desired length so as to improve or modulate binding of the ligand binding domain to the target cell, which may increase cytotoxic efficacy. In some embodiments, the linker or spacer between the ligand binding domain and the transmembrane can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

In some embodiments, the spacer comprises a CD8 hinge. In some embodiments, the spacer comprises a hinge region of a human antibody. In some embodiments, the spacer comprises an IgG4 hinge. In some embodiments, the IgG4 hinge region is a modified IgG4 hinge. In some embodiments, the IgG4 hinge region is a IgG4-CH3 hinge. In some embodiments, the IgG4 hinge region is a IgG4-CH2-CH3 hinge. A “modified IgG4 hinge” as described herein can refer to a hinge region that can have at least 90%, 91%, 92%, 93%, 94% 95%, 96%, 97%, 98%, 99%, or 100% sequence identity or a sequence identity within a range defined by any two of the aforementioned percentages, with a hinge region amino acid sequence as set forth in a spacer. In some embodiments, the CAR comprises a short (“S”) spacer, medium (“M”) spacer or a long (“L”) spacer. In some embodiments, a short spacer has a length in a range greater than or equal to 1 and less than or equal to 12 consecutive amino acids, or a length of 12 residues or about 12 residues. In some embodiments, a medium spacer has a length in a range from greater than or equal to 13 and less than or equal to 119 consecutive amino acids, or a length of 119 residues or about 119 residues. In some embodiments, a long spacer has a length in a range greater than or equal to 120 and less than or equal to 229 consecutive amino acids, or a length of 229 residues or about 229 residues.

As used herein, a “de-immunized spacer” has its plain and ordinary meaning when read in light of the specification, and includes, for example, a spacer that induces little to no immune response or a diminished or reduced immune response from a patient. In some embodiments, the CAR comprises a spacer, wherein the spacer does not induce an immune response in a subject, such as a human. It is important that the spacer does not induce an immune response or induces a reduced or diminished or low immune response in a subject, such as a human, in order to prevent or reduce the ability of the immune system to attack the chimeric antigen receptor.

In some embodiments, the transmembrane domain is a region of a membrane-spanning protein that is hydrophobic that can reside in the bilayer of a cell to anchor a protein that is embedded to the biological membrane. Without being limiting, the topology of the transmembrane domain can be a transmembrane alpha helix. In some embodiments, a CAR comprises a sequence encoding a transmembrane domain. In some embodiments, the transmembrane domain comprises a CD8 transmembrane sequence or a fragment thereof or a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some embodiments, the CD8 transmembrane sequence or a fragment thereof or the CD28 transmembrane sequence or fragment thereof comprises 28 amino acids in length.

In some embodiments, the signaling domains, such as primary signaling domains or costimulatory domains, include an intracellular or cytoplasmic domain of a protein or a receptor protein that interacts with components within the interior of the cells and is capable of or configured to relay or participate in the relaying of a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes one or more co-stimulatory domains. In some embodiments, the one or more costimulatory domains include a signaling moiety that provides a T-cell with a signal, which, in addition to the primary signal provided by for instance the CD3 zeta chain of the TCR/CD3 complex, enhances a response such as a T-cell effector response, such as, for example, an immune response, activation, proliferation, differentiation, cytokine secretion, cytolytic activity, perforin or granzyme activity or any combination thereof. In some embodiments, the intracellular signaling domain or the co-stimulatory domain can include all or a portion of CD3z, CD27, CD28, 4-1BB, OX40, CD30, CD40, ICOS, lymphocyte function-associated antigen-1 (LFA-1), CD2, CD7, LIGHT, NKG2C, B7-H3, or any combination thereof.

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some embodiments of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the embodiments described herein, the fusion protein provided can comprise a marker sequence that can be selected in experiments, such as flow cytometry. In some embodiments, the marker is a truncated EGFR polypeptide (EGFRt), or a truncated HER2 polypeptide (HER2t).

As used herein, a “signal sequence” for secretion, can also be referred to as a “signal peptide,” “leader sequence,” or “leader peptide.” The signal peptide can be used for secretion efficiency and in some systems, it is recognized by a signal recognition particle, which halts translation and directs the signal sequence to a SRP receptor for secretion. In some embodiments of the CARs provided herein, the CARs further comprise a signal sequence. In some embodiments, of the nucleic acid encoding a CAR, the nucleic acid comprises a sequence encoding a signal sequence. In some embodiments, the signal sequence is for targeting a protein to a cell membrane following translation of the protein.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “T-cells” or “T lymphocytes” can be from any mammal, preferably a primate, including monkeys or humans, a companion animal such as a dog, cat, or horse, or a domestic animal, such as a sheep, goat, or cattle. In some embodiments the T-cells are allogeneic (from the same species but different donor) as the recipient subject; in some embodiments the T-cells are autologous (the donor and the recipient are the same); in some embodiments the T-cells are syngeneic (the donor and the recipients are different but are identical twins).

As used herein, “T cell precursors” refer to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4−CD8−) cells. As they progress through their development, they become double-positive thymocytes (CD4+CD8+), and finally mature to single-positive (CD4+CD8− or CD4−CD8+) thymocytes that are then released from the thymus to peripheral tissues.

As used herein, “hematopoietic stem cells” or “HSC” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (LUM).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells have the ability to make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells have the ability to make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MHC class II, a CD4+ cell will aid those cells through a combination of cell-to-cell interactions (e.g. CD40 and CD40L) and through cytokines. Nevertheless, there are rare exceptions; for example, sub-groups of regulatory T-cells, natural killer cells, and cytotoxic T-cells express CD4. All of the latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof, and does not express or has decreased expression of CD45RA, as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells, and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to an antigen experienced cytotoxic T lymphocyte cells that do not express or have decreased expression of CD62L, CCR7, CD28, and are positive for granzyme B or perforin or both, as compared to central memory or naïve T-cells.

As used herein, “cytokines” has its plain and ordinary meaning when read in light of the specification, and includes, for example, small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, or tumor necrosis factor or any combination thereof. Cytokines can be produced by a broad range of cells, which can include, for example, immune cells like macrophages, B lymphocytes, T lymphocytes, mast cells, as well as endothelial cells, fibroblasts, or various stromal cells.

Cytokines can act through receptors and are important in the immune system as the cytokines can modulate the balance between humoral and cell-based immune responses, and they can regulate the maturation, growth, and responsiveness of particular cell populations. Some cytokines enhance or inhibit the action of other cytokines in complex ways. Without being limiting, cytokines can include, for example, Acylation stimulating protein, Adipokine, Albinterferon, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, Chemokine, Colony-stimulating factor, CX3CL1, CX3CR1, CXCL1, CXCL10, CXCLL1, CXCL13, CXCL14, CXCL15, CXCL16, CXCL17, CXCL2, CXCL3, CXCL5, CXCL6, CXCL7, CXCL9, Erythropoietin, Gc-MAF, Granulocyte colony-stimulating factor, Granulocyte macrophage colony-stimulating factor, Hepatocyte growth factor. IL 10 family of cytokines, IL 17 family of cytokines, IL1A, IL1B, Inflammasome, interferome, Interferon, Interferon beta 1a, Interferon beta 1b, Interferon gamma, Interferon type I, Interferon type II, Interferon type M, Interleukin, Interleukin 1 family, Interleukin 1 receptor antagonist, Interleukin 10, Interleukin 12, Interleukin 12 subunit beta, Interleukin 13, Interleukin 15, Interleukin 16, Interleukin 2, Interleukin 23, Interleukin 23 subunit alpha, Interleukin 34, Interleukin 35, Interleukin 6, Interleukin 7, Interleukin 8, Interleukin 36, Leukemia inhibitory factor, Leukocyte-promoting factor, Lymphokine, Lymphotoxin, Lymphotoxin alpha, Lymphotoxin beta, Macrophage colony-stimulating factor, Macrophage inflammatory protein, Macrophage-activating factor, Monokine, Myokine, Myonectin, Nicotinamide phosphoribosyltransferase, Oncostatin M, Oprelvekin, Platelet factor 4, Proinflammatory cytokine, Promegapoietin, RANKL, Stromal cell-derived factor 1, Talimogene laherparepvec, Tumor necrosis factor alpha, Tumor necrosis factors, XCL1, XCL2, GM-CSF, or XCR1 or any combination thereof. In some embodiments of the methods of making genetically modified T-cells, a transduced population of CD8+ expressing T-cells and/or CD4+ expressing T-cells is contacted with at least one cytokine so as to generate a transduced, cytokine-stimulated population of CD8+ T-cells and/or CD4+ T-cells. In some embodiments, the at least one cytokine utilized comprises GM-CSF, IL-7, IL-12, IL-15, IL-18, IL-2 or IL-21 or any combination thereof. In some embodiments, the period of contact with the cytokine is at least one day, such as for at least 1, 2, 3, 4, 5, 6, 7, 8, 9, 10, 11, 12, 13, 14, 15, 16, 17, 18, 19, or 20 days or any time that is within a range of times defined by any two of the aforementioned time points.

As used herein, “interleukins” or IL are cytokines that the immune system depends largely upon. Examples of interleukins, which can be utilized herein, for example, include IL-1, IL-2, IL-3, IL-4, IL-5, IL-6, 11-7, IL-8/CXCL8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36 or any combination thereof. Contacting T-cells with interleukins can have effects that promote, support, induce, or improve engraftment fitness of the cells. IL-1, for example can function in the maturation & proliferation of T-cells. IL-2, for example, can stimulate growth and differentiation of T-cell response. IL-3, for example, can promote differentiation and proliferation of myeloid progenitor cells. IL-4, for example, can promote proliferation and differentiation. IL-7, for example, can promote differentiation and proliferation of lymphoid progenitor cells, involved in B, T, and NK cell survival, development, and homeostasis. IL-15, for example, can induce production of natural killer cells. IL-21, for example, co-stimulates activation and proliferation of CD8+ T-cells, augments NK cytotoxicity, augments CD40-driven B cell proliferation, differentiation and isotype switching, and/or promotes differentiation of Th17 cells.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some embodiments of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some embodiments of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

As used herein, “genetically modified immune cells” or “Genetically engineered cells” are made by a process called genetic engineering, which can include but is not limited to manipulating a cell's own genome or inserting a new nucleic acid into a cell. In some embodiments, these cells can be macrophages and can also be referred to as genetically engineered macrophages (GEMs). These techniques can be used to change the genetic makeup of the cell, and can include inserting a vector encoding a gene of interest into a cell, and genome editing using RNAi systems, meganucleases, zinc finger nucleases, transcription activator like effector nucleases (TALENS), or CRISPRs. Without being limiting, the vectors encoding the gene of interest can be a viral vector, DNA or an mRNA. In some embodiments, described herein, genetically modified immune cells are provided. In some embodiments, the genetically modified immune cells are made using genome editing proteins or systems, such as for example, meganucleases, zinc finger nucleases, transcription activator like effector nucleases (TALENS), CRISPR-VP64-Cas9 systems or CRISPR/CAS9 systems.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

Some embodiments include polypeptide sequences or conservative variations thereof, such as conservative substitutions in a polypeptide sequence. In some embodiments, “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence | Gly, Pro |
| chain orientation | |
| aromatic | Trp, Tyr, Phe, His |

### Certain CARs

Some embodiments of the methods and compositions provided herein include a CAR, or nucleic acid encoding the CAR. In some embodiments, the CAR is capable of specifically binding to biotin and more particularly to conjugated biotin. In some embodiments, the CAR comprises a ligand binding domain capable of specifically binding to a conjugated biotin, a transmembrane domain, and an intracellular signaling domain.

In some embodiments, the ligand binding domain comprises a complementarity-determining region (CDR) derived from a binding moiety polypeptide, such as a binding moiety polypeptide capable of specifically binding to a conjugated biotin. In some embodiments, the ligand binding domain comprises an antigen-binding fragment of an antibody, an scFv, or a variable heavy chain (VH) domain and a variable heavy chain (VL) domain. In some embodiments, the VH domain and the VL domain are linked via a linker, such as a triple repeat of the amino acid sequence “GGGGS”. In some embodiments, the ligand binding domain comprises a VH-VL domains in which the VH domain precedes the VL domain in the polypeptide. In some embodiments, the ligand binding domain comprises a VL-VH domains in which the VL domain precedes the VH domain in the polypeptide.

In some embodiments, the ligand binding domain comprises, consists essentially of, or consists of a VH domain. In some embodiments, the ligand binding domain lacks a VL domain. In some embodiments, the ligand binding domain can consist essentially of a VH domain and no more than 10 other amino acids. In some embodiments, the ligand binding domain can consist essentially of a VH domain and no more than 5 other amino acids. In some embodiments, the ligand binding domain can consist essentially of a VH domain and no more than 3 other amino acids.

In some embodiments, the CAR is encoded by a polynucleotide in which the polynucleotide is operably linked to a promoter. In some embodiments, the promoter comprises a constitutive promoter. In some embodiments, the constitutive promoter comprises an EF1α promoter. In some embodiments, the promoter comprises an inducible promoter.

In some embodiments, the polynucleotide encoding the CAR also includes a nucleic acid encoding a cell-surface selectable marker. In some embodiments, the cell-surface selectable marker is selected from a truncated EGFR polypeptide (EGFRt) or a truncated Her2 polypeptide (Her2t). In some embodiments, the polynucleotide also includes a ribosome skip sequence located between a nucleic encoding the CAR and the nucleic acid encoding the cell-surface selectable marker. In some embodiments, the ribosome skip sequence is selected from the group consisting of a P2A sequence, a T2A sequence, an E2A sequence, and an F2A sequence.

Some embodiments of the methods and compositions provided herein include a vector comprising any one of the nucleic acids provided herein, such as a polynucleotide encoding a CAR. In some embodiments, the vector is a viral vector. In some embodiments, the vector is a lentiviral vector, foamy viral vector, retroviral vector, an adenoviral vector, or an adenovirus associated viral (AAV) vector. In some embodiments, the vector is a transposon, integrase vector system, or an mRNA vector.

In some embodiments, the CAR comprises: (i) an scFv domain configured to bind to conjugated biotin; (ii) a spacer domain; (iii) a transmembrane domain; and (iv) an intracellular signaling domain. In some embodiments, the scFv domain selectively binds to conjugated biotin compared to biotin not conjugated to a molecule (free biotin).

In some embodiments, the scFv domain comprises a VH domain and a VL domain.

In some embodiments, the VH domain comprises: (a) a heavy chain hyper variable region 1 (HVR-H1) comprising the amino acid sequence of SEQ ID NO: 27 comprising 0-3 conservative substitutions thereof; (b) a HVR-H2 comprising the amino acid sequence of SEQ ID NO: 28 comprising 0-3 conservative substitutions thereof; and/or (c) a HVR-H3 comprising the amino acid sequence of SEQ ID NO: 29 comprising 0-3 conservative substitutions thereof. In some embodiments, the VH domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%,a, or 100% sequence identity to the amino acid sequence of SEQ ID NO: 1.

In some embodiments, the VL domain comprises: (a) a light chain hyper variable region 1 (HVR-L1) comprising the amino acid sequence of SEQ ID NO: 30 comprising 0-3 conservative substitutions thereof; (b) a HVR-L2 comprising the amino acid sequence of SEQ ID NO: 31 comprising 0-3 conservative substitutions thereof; and/or (c) a HVR-L3 comprising the amino acid sequence of SEQ ID NO: 32 comprising 0-3 conservative substitutions thereof. In some embodiments, the VL domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 2.

In some embodiments, the VH domain is located in the scFv N-terminal of the VL domain. In some embodiments, the VL domain is located in the scFv N-terminal of the VH domain. In some embodiments, the VH domain and the VL domain are joined via a linker domain. In some embodiments, the linker domain comprises or consists of the amino acid sequence of SEQ ID NO:23. In some embodiments, the scFv domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 23 or 24.

In some embodiments, the intracellular signaling domain comprises a 4-1BB domain, a CD3-zeta domain, and/or a CD28tm domain. In some embodiments, the 4-1BB domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%/o, or 100% sequence identity to SEQ ID NO: 8. In some embodiments, the CD3-zeta domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the CD28tm domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 4.

In some embodiments, the spacer domain comprises an IgG4 hinge domain, and IgG4 hinge-CH3 domain, or an IgG4 hinge -CH2-CH3 domain. In some embodiments, the spacer domain has a length: (i) in a range greater than or equal to 1 and less than or equal to 12 consecutive amino acids, or a length of 12 residues or about 12 residues; (ii) in a range from greater than or equal to 13 and less than or equal to 119 consecutive amino acids, or a length of 119 residues or about 119 residues; or (iii) in a range greater than or equal to 120 and less than or equal to 229 consecutive amino acids, or a length of 229 residues or about 229 residues. In some embodiments, the spacer domain comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of any one of SEQ ID NOs: 12, 13 or 14.

In some embodiments, the CAR comprises an amino acid sequence having at least or at least about 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 15-20. In some embodiments, the CAR further comprises a signal peptide.

Certain sequences useful with embodiments provided herein are listed in TABLE 2.

TABLE 2

| Feature | |
|---|---|
| SEQ ID NO | Sequence |
| VH | QVQLVQSGAEVKKPGSSVKVSCKSSGFNNKDTFFQWVRQAPG |
| SEQ ID NO: 1 | QGLEWMGRIDPANGFTKYAQKFQGRVTITADTSTSTAYMELSSL |
| | RSEDTAVYYCARWDTYGAAWFAYWGQGTLVTVSS |
| VL | DIQMTQSPSSLSASVGDRVTITCRASGNIHNYLSWYQQKPGKVP |
| SEQ ID NO: 2 | KLLIYSAKTLADGVPSRFSGSGSGTDFTLTISSLQPEDVATYYCQ |
| | HFWSSIYTFGQGTKLEIK |
| Signal/leader | MLLLVTSLLLCELPHPAFLLIP |
| peptide (GM- | |
| CSF) | |
| SEQ ID NO: 3 | |
| CD28tm | MFWVLVVVGGVLACYSLLVTVAFIIFWV |
| SEQ ID NO: 4 | |
| CD28 linker | IEVMYPPPYLDNEKSNGTIIHVKGKHLCPSPLFPGPSK |
| SEQ ID NO: 5 | |
| CD28 linker and | IEVMYPPPYLDNEKSNGTIIHVKGKHLCPSPLFPGPSKPFWVLV |
| transmembrane | VVGGVLACYSLLVTVAFIIFWV |
| domain | |
| SEQ ID NO: 6 | |
| CD28 | RSKRSRLLHSDYMNMTPRRPGPTRKHYQPYAPPRDFAAYRS |
| cytoplasmic | |
| SEQ ID NO: 7 | |
| 4-1BB signaling | KRGRKKLLYIFKQPFMRPVQTTQBEDGCSCRFPEEEEGGCEL |
| domain | |
| SEQ ID NO: 8 | |
| CD3-zeta chain | RVKFSRSADAPAYQQGQNQLYNELNLGRREEYDVLDKRRGRD |
| signaling | PEMGGKPRRKNPQEGLYNELQKDKMAEAYSEIGMKGERRRGK |
| domain | GHDGLYQGLSTATKDTYDALHMQALPPR |
| SEQ ID NO: 9 | |
| T2A | GGGEGRGSLLTCGDVEENPGP |
| SEQ ID NO: 10 | |
| EGFRt | RKVCNGIGIGEFKDSLSINATNIKHFKNCTSISGDLHILPVAFRG |
| SEQ ID NO: 11 | DSFTHTPPLDPQELDILKTVKEITGFLLIQAWPENRTDLHAFENL |
| | EIIRGRTKQHGQFSLAVVSLNITSLGLRSLKEISDGDVIISGNKNL |
| | CYANTINWKKLFGTSGQKTKIISNRGENSCKATGQVCHALCSP |
| | EGCWGPEPRDCVSCRNVSRGRECVDKCNLLEGEPREFVENSEC |
| | IQCHPECLPQAMNITCTGRGPDNCIQCAHYIDGPHCVKTCPAGV |
| | MGENNTLVWKYADAGHVCHLCHPNCTYGCTGPGLEGCPTNG |
| | PKIPSIATGMVGALLLLLVVALGIGLFM |
| S (small) spacer | ESKYGPPCPPCP |
| SEQ ID NO: 12 | |
| M (medium) spacer | ESKYGPPCPPCPGQPREPQVYTLPPSQEEMTKNQVSLTCLVKGF |
| SEQ ID NO: 13 | YPSDIAVEWESNGQPENNYKTTPPVLDSDGSFFLYSRLTVDKSR |
| | WQEGNVFSCSVMHEALHNHYTQKSLSLSLGK |
| L (long) spacer | ESKYGPPCPPCPAPEFDGGPSVFLFPPKPKDTLMISRTPEVTCVV |
| SEQ ID NO: 14 | VDVSQEDPEVQFNWYVDGVEVHNAKTKPREEQFQSTYRVVSV |
| | LTVLHQDWLNGKEYKCKVSNKGLPSSIEKTISKAKGQPREPQV |
| | YTLPPSQEEMTKNQVSLTCLVKGFYPSDIAVEWESNGQPENNY |
| | KTTPPVLDSDGSFFLYSRLTVDKSRWQEGNVFSCSVMHEALHN |
| | HYTQKSLSLSLGK |
| VHVL “S” | MLLLVTSLLLCELPHPAFLLIPQVQLVQSGAEVKKPGSSVKVSC |
| Construct | KSSGFNNKDTFFQWVRQAPGQGLEWMGRIDPANGFTKYAQKF |
| SEQ ID NO: 15 | QGRVTITADTSTSTAYMELSSLRSEDTAVYYCARWDTYGAAW |
| | FAYWGQGTLVTVSSGGGGSGGGGSGGGGSDIQMTQSPSSLSAS |
| | VGDRVTITCRASGNIHNYLSWYQQKPGKVPKLLIYSAKTLADG |
| | VPSRFSGSGSGTDFTLTISSLQPEDVATYYCQHFWSSIYTFGQGT |
| | KLEIKESKYGPPCPPCPMFWVLVVVGGVLACYSLLVTVAFIIFW |
| | VKRGRKKLLYIFKQPFMRPVQTTQEEDGCSCRFPEEEEGGCELR |
| | VKFSRSADAPAYQQGQNQLYNELNLGRREEYDVLDKRRGRDP |
| | EMGGKPRRKNPQEGLYNELQKDKMAEAYSEIGMKGERRRGK |
| | GHDGLYQGLSTATKDTYDALHMQALPPRGSGATNFSLLKQAG |
| | DVEENPGPMVGSLNCIVAVSQNMGIGKNGDFPWPPLRNESRYF |
| | QRMTTTSSVEGKQNLVIMGKKTWFSIPEKNRPLKGRINLVLSRE |
| | LKEPPQGAHFLSRSLDDALKLTEQPELANKVDMVWIVGGSSVY |
| | KEAMNHPGHLKLFVTRIMQDFESDTFFPEIDLEKYKLLPEYPGV |
| | LSDVQEEKGIKYKFEVYEKNDGGGEGRGSLLTCGDVEENPGPM |
| | LLLVTSLLLCELPHPAFLLIPRKVCNGIGIGEFKDSLSINATNIKH |
| | FKNCTSISGDLHILPVAFRGDSFTHTPPLDPQELDILKTVKEITGF |
| | LLIQAWPENRTDLHAFENLEIIRGRTKQHGQFSLAVVSLNITSLG |
| | LRSLKEISDGDVIISGNKNLCYANTINWKKLFGTSGQKTKIISNR |
| | GENSCKATGQVCHALCSPEGCWGPEPRDCVSCRNVSRGRECV |
| | DKCNLLEGEPREFVENSECIQCHPECLPQAMNITCTGRGPDNCI |
| | QCAHYIDGPHCVKTCPAGVMGENNTLVWKYADAGHVCHLCH |
| | PNCTYGCTGPGLEGCPTNGPKIPSIATGMVGALLLLLVVALGIG |
| | LFM |
| VHVL “M” | MLLLVTSLLLCELPHPAFLLIPQVQLVQSGAEVKKPGSSVKVSC |
| Construct | KSSGFNNKDTFFQWVRQAPGQGLEWMGRIDPANGFTKYAQKF |
| SEQ ID NO: 16 | QGRVTITADTSTSTAYMELSSLRSEDTAVYYCARWDTYGAAW |
| | FAYWGQGTLVTVSSGGGGSGGGGSGGGGSDIQMTQSPSSLSAS |
| | VGDRVTITCRASGNIHNYLSWYQQKPGKVPKLLIYSAKTLADG |
| | VPSRFSGSGSGTDFTLTISSLQPEDVATYYCQHFWSSIYTFGQGT |
| | KLEIKESKYGPPCPPCPGQPREPQVYTLPPSQEEMTKNQVSLTC |
| | LVKGFYPSDIAVEWESNGQPENNYKTTPPVLDSDGSFFLYSRLT |
| | VDKSRWQEGNVFSCSVMHEALHNHYTQKSLSLSLGKMFWVL |
| | VVVGGVLACYSLLVTVAFIIFWVKRGRKKLLYIFKQPFMRPVQ |
| | TTQEEDGCCRFPEEEEGGCELRVKFSRSADAPAYQQGQNQLY |
| | NELNLGRREEYDVLDKRRGRDPEMGGKPRRKNPQEGLYNELQ |
| | KDKMAEAYSEIGMKGERRRGKGHDGLYQGLSTATKDTYDAL |
| | HMQALPPRGSGATNFSLLKQAGDVEENPGPMVGSLNCIVAVSQ |
| | NMGIGKNGDFPWPPLRNESRYFORMTTTSSVEGKONLVIMGK |
| | KTWFSIPEKNRPLKGRINLVLSRELKEPPQGAHFLSRSLDDALK |
| | LTEQPELANKVDMVWIVGGSSVYKEAMNHPGHLKLFVTRIMQ |
| | DFESDTFFPEIDLEKYKLLPEYPGVLSDVQEEKGIKYKFEVYEK |
| | NDGGGEGRGSLLTCGDVEENPGPMLLLVTSLLLCELPHPAFLLI |
| | PRKVCNGIGIGEFKDSLSINATNIKHFKNCTSISGDLHILPVAFRG |
| | DSFTHTPPLDPQELDILKTVKEITGFLLIQAWPENRTDLHAFENL |
| | EIIRGRTKQHGQFSLAVVSLNITSLGLRSLKEISDGDVIISGNKNL |
| | CYANTINWKKLFGTSGQKTKIISNRGENSCKATGQVCHALCSP |
| | EGCWGPEPRDCVSCRNVSRGRECVDKCNLLEGEPREFVENSEC |
| | IQCHPECLPQAMNITCTGRGPDNCIQCAHYIDGPHCVKTCPAGV |
| | MGENNTLVWKYADAGHVCHLCHPNCTYGCTGPGLEGCPTNG |
| | PKIPSIATGMVGALLLLLVVALGIGLFM |
| VHVL “L” | MLLLVTSLLLCELPHPAFLLIPQVQLVQSGAEVKKPGSSVKVSC |
| Construct | KSSGFNNKDTFFQWVRQAPGQGLEWMGRIDPANGFTKYAQKF |
| SEQ ID NO: 17 | QGRVTITADTSTSTAYMELSSLRSEDTAVYYCARWDTYGAAW |
| | FAYWGQGTLVTVSSGGGGGGGGSGGGGSDIQMTQSPSSLSAS |
| | VGDRVTITCRASGNIHNYLSWYQQKPGKVPKLLIYSAKTLADG |
| | VPSRFSGSGSGTDFTLTISSLOPEDVATYYCQHFWSSIYTFGQGT |
| | KLEIKESKYGPPCPPCPAPEFDGGPSVFLFPPKPKDTLMISRTPEV |
| | TCVVVDVSQEDPEVQFNWYVDGVEVHNAKTKPREEQFQSTYR |
| | VVSVLTVLHQDWLNGKEYKCKVSNKGLPSSIEKTISKAKGQPR |
| | EPQVYTLPPSQEEMTKNQVSLTCLVKGFYPSDIAVEWESNGOP |
| | ENNYKTTPPVLDSDGSFFLYSRLTVDKSRWQEGNVFSCSVMHE |
| | ALHNHYTQKSLSLSLGKMFWVLVVVGGVLACYSLLVTVAFIIF |
| | WVKRGRKKLLYIFKQPFMRPVQTTQEEDGCSCRFPEEEEGGCE |
| | LRVKFSRSADAPAYQQGQNQLYNELNLGRREEYDVLDKRRGR |
| | DPEMGGKPRRKNPQEGLYNELQKDKMAEAYSEIGMKGERRRG |
| | KGHDGLYQGLSTATKDTYDALHMQALPPRGSGATNFSLLKQA |
| | GDVEENPGPMVGSLNCIVAVSQNMGIGKNGDFPWPPLRNESRY |
| | FORMTTTSSVEGKONLVIMGKKTWFSIPEKNRPLKGRINLVLSR |
| | ELKEPPQGAHFLSRSLDDALKLTEQPELANKVDMVWIVGGSSV |
| | YKEAMNHPGHLKLFVTRIMQDFESDTFFPEIDLEKYKLLPEYPG |
| | VLSDVQEEKGIKYKFEVYEKNDGGGEGRGSLLTCGDVEENPGP |
| | MLLLVTSLLLCELPHPAFLLIPRKVCNGIGIGEFKDSLSINATNIK |
| | HFKNCTSISGDLHILPVAFRGDSFTHTPPLDPQELDILKTVKEITG |
| | FLLIQAWPENRTDLHAFENLEIIRGRTKQHGQFSLAVVSLNITSL |
| | GLRSLKEISDGDVIISGNKNLCYANTINWKKLFGTSGQKTKIISN |
| | RGENSCKATGQVCHALCSPEGCWGPEPRDCVSCRNVSRGREC |
| | VDKCNLLEGEPREFVENSECIQCHPECLPQAMNITCTGRGPDNC |
| | IQCAHYIDGPHCVKTCPAGVMGENNTLVWKYADAGHVCHLC |
| | HPNCTYGCTGPGLEGCPTNGPKIPSIATGMVGALLLLLVVALGI |
| | GLFM |
| VLVH “S” | MLLLVTSLLLCELPHPAFLLIPDIQMTQSPSSLSASVGDRVTITC |
| Construct | RASGNIHNYLSWYQQKPGKVPKLLIYSAKTLADGVPSRFSGSG |
| SEQ ID NO: 18 | SGTDFTLTISSLQPEDVATYYCQHFWSSIYTFGQGTKLEIKGGG |
| | GSGGGGSGGGGSQVQLVQSGAEVKKPGSSVKVSCKSSGFNNK |
| | DTFFQWVRQAPGOGLEWMGRIDPANGFTKYAQKFQGRVTITA |
| | DTSTSTAYMELSSLRSEDTAVYYCARWDTYGAAWFAYWGQG |
| | TLVTVSSESKYGPPCPPCPMFWVLVVVGGVLACYSLLVTVAFII |
| | FWVKRGRKKLLYIFKQPFMRPVQTTQEEDGCSCRFPEEEEGGC |
| | ELRVKFSRSADAPAYQQGQNQLYNELNLGRREEYDVLDKRRG |
| | RDPEMGGKPRRKNPQEGLYNELQKDKMAEAYSEIGMKGERRR |
| | GKGHDGLYQGLSTATKDTYDALHMQALPPRGSGATNFSLLKQ |
| | AGDVEENPGPMVGSLNCIVAVSQNMGIGKNGDFPWPPLRNESR |
| | YFORMTTTSSVEGKQNLVIMGKKTWFSIPEKNRPLKGRINLVLS |
| | RELKEPPQGAHFLSRSLDDALKLTEQPELANKVDMVWIVGGSS |
| | VYKEAMNHPGHLKLFVTRIMQDFESDTFFPEIDLEKYKLLPEYP |
| | GVLSDVQEEKGIKYKFEVYEKNDGGGEGRGSLLTCGDVEENPG |
| | PMLLLVTSLLLCELPHPAFLLIPRKVCNGIGIGEFKDSLSINATNI |
| | KHFKNCTSISGDLHILPVAFRGDSFTHTPPLDPQELDILKTVKEIT |
| | GFLLIQAWPENRTDLHAFENLEIIRGRTKQHGOFSLAVVSLNITS |
| | LGLRSLKEISDGDVIISGNKNLCYANTINWKKLFGTSGQKTKIIS |
| | NRGENSCKATGQVCHALCSPEGCWGPEPRDCVSCRNVSRGRE |
| | CVDKCNLLEGEPREFVENSECIQCHPECLPQAMNITCTGRGPDN |
| | CIQCAHYIDGPHCVKTCPAGVMGENNTLVWKYADAGHVCHL |
| | CHPNCTYGCTGPGLEGCPTNGPKIPSIATGMVGALLLLLVVALG |
| | IGLFM |
| VLVH “M” | MLLLVTSLLLCELPHPAFLLIPDIQMTQSPSSLSASVGDRVTITC |
| Construct | RASGNIHNYLSWYQQKPGKVPKLLIYSAKTLADGVPSRFSGSG |
| SEQ ID NO: 19 | SGTDFTLTISSLOPEDVATYYCQHFWSSIYTFGQGTKLEIKGGG |
| | GSGGGGSGGGGSQVQLVQSGAEVKKPGSSVKVSCKSSGFNNK |
| | DTFFQWVRQAPGQGLEWMGRIDPANGFTKYAQKFQGRVTITA |
| | DTSTSTAYMELSSLRSEDTAVYYCARWDTYGAAWFAYWGQG |
| | TLVTVSSESKYGPPCPPCPGQPREPQVYTLPPSQEEMTKNQVSL |
| | TCLVKGFYPSDIAVEWESNGQPENNYKTTPPVLDSDGSFFLYSR |
| | LTVDKSRWQEGNVFSCSVMHEALHNHYTQKSLSLSLGKMFW |
| | VLVVVGGVLACYSLLVTVAFIIFWVKRGRKKLLYIFKQPFMRP |
| | VQTTQEEDGCSCRFPEEEEGGCELRVKFSRSADAPAYQQGQNQ |
| | LYNELNLGRREEYDVLDKRRGRDPEMGGKPRRKNPQEGLYNE |
| | LQKDKMAEAYSEIGMKGERRRGKGHDGLYQGLSTATKDTYD |
| | ALHMQALPPRGSGATNFSLLKQAGDVEENPGPMVGSLNCIVA |
| | VSQNMGIGKNGDFPWPPLRNESRYFQRMTTTSSVEGKQNLVIM |
| | GKKTWFSIPEKNRPLKGRINLVLSRELKEPPQGAHFLSRSLDDA |
| | LKLTEQPELANKVDMVWIVGGSSVYKEAMNHPGHLKLFVTRI |
| | MQDFESDTFFPEIDLEKYKLLPEYPGVLSDVQEEKGIKYKFEVY |
| | EKNDGGGEGRGSLLTCGDVEENPGPMLLLVTSLLLCELPHPAF |
| | LLIPRKVCNGIGIGEFKDSLSINATNIKHFKNCTSISGDLHILPVA |
| | FRGDSFTHTPPLDPQELDILKTVKEITGFLLIQAWPENRTDLHAF |
| | ENLEIIRGRTKQHGQFSLAVVSLNITSLGLRSLKEISDGDVIISGN |
| | KNLCYANTINWKKLFGTSGQKTKIISNRGENSCKATGQVCHAL |
| | CSPEGCWGPEPRDCVSCRNVSRGRECVDKCNLLEGEPREFVEN |
| | SECIQCHPECLPQAMNITCTGRGPDNCIQCAHYIDGPHCVKTCP |
| | AGVMGENNTLVWKYADAGHVCHLCHPNCTYGCTGPGLEGCP |
| | TNGPKIPSIATGMVGALLLLLVVALGIGLFM |
| VLVH “L” | MLLLVTSLLLCELPHPAFLLIPDIQMTQSPSSLSASVGDRVTITC |
| Construct | RASGNIHNYLSWYQQKPGKVPKLLIYSAKTLADGVPSRFSGSG |
| SEQ ID NO: 20 | SGTDFTLTISSLOPEDVATYYCQHFWSSIYTFGQGTKLEIKGGG |
| | GSGGGGSGGGGSQVQLVQSGAEVKKPGSSVKVSCKSSGFNNK |
| | DTFFQWVRQAPGOGLEWMGRIDPANGFTKYAQKFOGRVTITA |
| | DTSTSTAYMELSSLRSEDTAVYYCARWDTYGAAWFAYWGQG |
| | TLVTVSSESKYGPPCPPCPAPEFDGGPSVFLFPPKPKDTLMISRT |
| | PEVTCVVVDVSQEDPEVQFNWYVDGVEVHNAKTKPREEQFQS |
| | TYRVVSVLTVLHQDWLNGKEYKCKVSNKGLPSSIEKTISKAKG |
| | QPREPQVYTLPPSQEEMTKNQVSLTCLVKGFYPSDIAVEWESN |
| | GQPENNYKTTPPVLDSDGSFFLYSRLTVDKSRWQEGNVFSCSV |
| | MHEALHNHYTQKSLSLSLGKMFWVLVVVGGVLACYSLLVTV |
| | AFIIFWVKRGRKKLLYIFKQPFMRPVQTTQEEDGCSCRFPEEEE |
| | GGCELRVKFSRSADAPAYQQGQNQLYNELNLGRREEYDVLDK |
| | RRGRDPEMGGKPRRKNPQEGLYNELQKDKMAEAYSEIGMKGE |
| | RRRGKGHDGLYQGLSTATKDTYDALHMQALPPRGSGATNFSL |
| | LKQAGDVEENPGPMVGSLNCIVAVSQNMGIGKNGDFPWPPLR |
| | NESRYFORMTTTSSVEGKQNLVIMGKKTWFSIPEKNRPLKGRIN |
| | LVLSRELKEPPQGAHFLSRSLDDALKLTEQPELANKVDMVWIV |
| | GGSSVYKEAMNHPGHLKLFVTRIMQDFESDTFFPEIDLEKYKLL |
| | PEYPGVLSDVQEEKGIKYKFEVYEKNDGGGEGRGSLLTCGDVE |
| | ENPGPMLLLVTSLLLCELPHPAFLLIPRKVCNGIGIGEFKDSLSIN |
| | ATNIKHFKNCTSISGDLHILPVAFRGDSFTHTPPLDPQELDILKT |
| | VKEITGFLLIQAWPENRTDLHAFENLEIIRGRTKQHGQFSLAVVS |
| | LNITSLGLRSLKEISDGDVIISGNKNLCYANTINWKKLFGTSGQK |
| | TKIISNRGENSCKATGQVCHALCSPEGCWGPEPRDCVSCRNVS |
| | RGRECVDKCNLLEGEPREFVENSECIQCHPECLPQAMNITCTGR |
| | GPDNCIQCAHYIDGPHCVKTCPAGVMGENNTLVWKYADAGH |
| | VCHLCHPNCTYGCTGPGLEGCPTNGPKIPSIATGMVGALLLLLV |
| | VALGIGLFM |
| DHFRdm | MVGSLNCIVAVSQNMGIGKNGDFPWPPLRNESRYFQRMTTTSS |
| SEQ ID NO: 21 | VEGKONLVIMGKKTWFSIPEKNRPLKGRINLVLSRELKEPPQGA |
| | HFLSRSLDDALKLTEQPELANKVDMVWIVGGSSVYKEAMNHP |
| | GHLKLFVTRIMQDFESDTFFPEIDLEKYKLLPEYPGVLSDVQEE |
| | KGIKYKFEVYEKND |
| P2A | GSGATNFSLLKQAGDVEENPGP |
| SEQ ID NO: 22 | |
| scFv (VHVL) | QVQLVQSGAEVKKPGSSVKVSCKSSGFNNKDTFFQWVRQAPG |
| SEQ ID NO: 23 | QGLEWMGRIDPANGFTKYAQKFQGRVTITADTSTSTAYMELSS |
| | LRSEDTAVYYCARWDTYGAAWFAYWGQGTLVTVSSGGGGSG |
| | GGGSGGGGSDIQMTQSPSSLSASVGDRVTITCRASGNIHNYLSW |
| | YQQKPGKVPKLLIYSAKTLADGVPSRFSGSGSGTDFTLTISSLQP |
| | EDVATYYCQHFWSSIYTFGQGTKLEIK |
| scFv (VLVH) | DIQMTQSPSSLSASVGDRVTITCRASGNIHNYLSWYQQKPGKVP |
| SEQ ID NO: 24 | KLLIYSAKTLADGVPSRFSGSGSGTDFTLTISSLQPEDVATYYCQ |
| | HFWSSIYTFGQGTKLEIKGGGGSGGGGSGGGGSQVQLVQSGAE |
| | VKKPGSSVKVSCKSSGFNNKDTFFQWVRQAPGQGLEWMGRID |
| | PANGFTKYAQKFQGRVTITADTSTSTAYMELSSLRSEDTAVYY |
| | CARWDTYGAAWFAYWGQGTLVTVSS |
| EF1 promoter | AGGCTCCGGTGCCCGTCAGTGGGCAGAGCGCACATCGCCCA |
| SEQ ID NO: 25 | CAGTCCCCGAGAAGTTGGGGGGAGGGGTCGGCAATTGAACC |
| | GGTGCCTAGAGAAGGTGGCGCGGGGTAAACTGGGAAAGTGA |
| | TGTCGTGTACTGGCTCCGCCTTTTTCCCGAGGGTGGGGGAGA |
| | ACCGTATATAAGTGCAGTAGTCGCCGTGAACGTTCTTTTTCG |
| | CAACGGGTTTGCCGCCAGAACACA |
| Linker | GGGGSGGGGSGGGGS |
| SEQ ID NO: 26 | |
| HVR-H1 | DTFFQ |
| SEQ ID NO: 27 | |
| HVR-HI | RIDPANGFTKYAQKFQ |
| SEQ ID NO: 28 | |
| HVR-H1 | WDTYGAAWFAY |
| SEQ ID NO: 29 | |
| HVR-LI | RASGNIHNYLS |
| SEQ ID NO: 30 | |
| HVR-L2 | SAKTLAD |
| SEQ ID NO: 31 | |
| HVR-L3 | QHFWSSIYT |
| SEQ ID NO: 32 | |

### Certain Cells Comprising CARs

Some embodiments of the methods and compositions provided herein include a cell, such as an immune cell, comprising any one of the CARs provided herein, or a nucleic acid encoding the CAR.

In some embodiments, the immune cell comprises a cell-surface selectable marker. In some embodiments, the cell-surface selectable marker is selected from a truncated EGFR polypeptide (EGFRt) or a truncated Her2 polypeptide (Her2t). In some embodiments, the immune cell comprises a suicide gene system. In some embodiments, the suicide gene system is selected from a herpes simplex virus thymidine kinase/ganciclovir (HSVTK/GCV) suicide gene system, or an inducible caspase suicide gene system.

In some embodiments, the immune cell is selected from a T cell, a natural killer cell, and a cell derived from a hematopoietic stem cell. In some embodiments, the immune cell is a CD4+ T-cell or a CD8+ T-cell. In some embodiments, the immune cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell. In some embodiments, the immune cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell. In some embodiments, the immune cell is mammalian. In some embodiments, the immune cell is human. In some embodiments, the immune cell is autologous to a subject, such as a recipient of an immunotherapy, such as a CAR T therapy. In some embodiments, the immune cell is allogeneic to a subject, such as a recipient of an immunotherapy, such as a CAR T therapy.

### Pharmaceutical Compositions

Some embodiments of the methods and compositions provided herein include a pharmaceutical composition comprising a cell comprising a CAR specific for a conjugated biotin, such as conjugated biotin as described in any one of the embodiments herein, or a nucleic acid encoding a CAR specific for conjugated biotin as described in any one of the embodiments herein, and a pharmaceutically acceptable excipient. Some embodiments include a pharmaceutical composition comprising a conjugated biotin provided herein and a pharmaceutically acceptable excipient. Some embodiments include a pharmaceutical composition comprising an immunoconjugate provided herein and a pharmaceutically acceptable excipient.

### Certain Therapies

Some embodiments of the methods and compositions provided herein include methods for inhibiting, ameliorating, or treating a cancer or a viral infection in a subject. In some embodiments, the cancer comprises a solid tumor or a leukemia. Some such methods include administering to the subject a cell comprising an anti-conjugated biotin CAR, such as an immune cell, of any one of the embodiments provided herein. In some embodiments the cell is administered in combination with a therapeutic compound, such as a conjugated biotin.

In some embodiments, the therapeutic compound, such as a conjugated biotin, is capable of specifically binding to a cell comprising the disorder. In some such embodiments, the conjugated biotin labels the cell comprising the disorder as a target cell which may be targeted by an effector cell, such as a cell comprising an anti-conjugated biotin CAR.

In some embodiments, the conjugated biotin is capable of specifically binding to a cancer cell. In some, embodiments, the conjugated biotin is capable of specifically binding to a cancer antigen. Examples of conjugated biotins include biotin conjugated to molecules such as a folate, a folate derivative, a phospholipid ether (PLE), an antibody capable of binding to a cancer antigen or an antigen-binding fragment thereof, such as an anti-CD19 antibody or antigen binding fragment thereof.

In some embodiments, the therapeutic compound is selected from: (i) an inhibitor of differentiation of a macrophage to an M2-type macrophage; (ii) a myeloid derived suppressor cell inhibitor; (iii) a colony stimulating factor-1 receptor (CSF-1R) kinase inhibitor, such as PLX3397, Pexidartinib, ARRY-382, JNJ-40346527, BLZ945, Emactuzumab, AMG820, or IMC-CS4; (iv) a macrophage migration inhibitory factor (MIF) inhibitor; (v) a transforming growth beta receptor-1 (TGFBR1) inhibitor, such as SD208; an indolamine 2,3-dioxyganse (IDO1) inhibitor, such as Epacadostat, navoximod, 1-methyl-d-tryptophan, or BMS-986205, or a COX-2 inhibitor; (vi) an inducible nitric oxide synthase (iNOS) inhibitor, such as N(G)-Nitro-L-arginine methyl ester (L-NAME), nitroarginine (L-NOARG), 4-amino-tetrahydrobiopterin (4-ABH4), cindunistat, A-84643, ONO-1714, NCX-456, VAS-2381, GW-273629, NXN-462, CKD-712, KD-7040, or guanidinoethyldisulfide; (vii) a PD-1 inhibitor, such as an anti-PD-1 antibody or antigen binding fragment thereof; (viii) an inducer of myeloid cell differentiation, such as all-trans retinoic acid (ATRA); or (ix) a combination of any one or more of the foregoing therapeutic compounds In some embodiments, the therapeutic compound is selected from ATRA, PLX3397, L-NAME, SD208, Epacadostat, an anti-PD-1 antibody or antigen binding fragment thereof, or a combination thereof.

In some embodiments, the cell, such as an immune cell, is administered to the subject prior to administration of the therapeutic compound. In some embodiments, the immune cell is administered to the subject at least or not more than 1, 2, 3, 4, 5, 6, 7, 8, 9, 10 day(s) prior to administration of the therapeutic compound. In some embodiments, the immune cell is administered to the subject concurrently with administration of the therapeutic compound. In some embodiments, the immune cell is administered to the subject subsequent to administration of the therapeutic compound. In some embodiments, the immune cell is administered to the subject at least or not more than 1, 2, 3, 4, 5, 6, 7, 8, 9, 10 day(s) subsequent to administration of the therapeutic compound.

In some embodiments, the subject is administered a single dose of the immune cell. In some embodiments, the subject is administered at least or not more than 1, 2, 3, 4, 5 doses of the immune cell. In some embodiments, the subject is administered more than a single dose of the therapeutic compound. In some embodiments, the subject is administered at least or not more than 1, 2, 3, 4, 5 doses of the therapeutic compound.

Some embodiments of the methods and compositions provided herein include methods the use of an immunoconjugate provided herein. Some embodiments of the methods and compositions provided herein include methods for inhibiting, ameliorating, or treating a cancer or a viral infection in a subject. In some embodiments, the cancer comprises a solid tumor or a leukemia. Some such methods include administering to the subject a conjugated biotin, such as a compound capable of targeting a cancer cell. Examples of conjugated biotins include biotin conjugated to molecules such as a folate, a folate derivative, a phospholipid ether (PLE), an antibody capable of binding to a cancer antigen or an antigen-binding fragment thereof, such as an anti-CD19 antibody or antigen binding fragment thereof. Some embodiments include administering to the subject comprising the compound, an immunoconjugate provided herein to target the conjugated biotin

### Certain Systems and Kits

Some embodiments of the methods and compositions provided herein include systems or kits for inhibiting, ameliorating, or treating a solid tumor in a subject. Some such systems include an immune cell of any one of the embodiments provided herein, and a therapeutic compound, such as a conjugated biotin. In some embodiments, the conjugated biotin includes biotin conjugated to molecules such as a folate, a folate derivative, a phospholipid ether (PLE), an antibody capable of binding to a cancer antigen or an antigen-binding fragment thereof. Some embodiments include an immunoconjugate provided herein.

In some embodiments, the therapeutic compound is selected from: (i) an inhibitor of differentiation of a macrophage to an M2-type macrophage; (ii) a myeloid derived suppressor cell inhibitor; (iii) a colony stimulating factor-1 receptor (CSF-1R) kinase inhibitor, such as PLX3397, Pexidartinib, ARRY-382, JNJ-40346527, BLZ945, Emactuzumab, AMG820, or IMC-CS4; (iv) a macrophage migration inhibitory factor (MIF) inhibitor; (v) a transforming growth beta receptor-1 (TGFBR1) inhibitor, such as SD208; an indolamine 2,3-dioxyganse (IDO1) inhibitor, such as Epacadostat, navoximod, 1-methyl-d-tryptophan, or BMS-986205, or a COX-2 inhibitor; (vi) an inducible nitric oxide synthase (iNOS) inhibitor, such as N(G)-Nitro-L-arginine methyl ester (L-NAME), nitroarginine (L-NOARG), 4-amino-tetrahydrobiopterin (4-ABH4), cindunistat, A-84643, ONO-1714, NCX-456, VAS-2381, GW-273629, NXN-462, CKD-712, KD-7040, or guanidinoethyldisulfide; (vii) a PD-1 inhibitor, such as an anti-PD-1 antibody or antigen binding fragment thereof; (viii) an inducer of myeloid cell differentiation, such as all-trans retinoic acid (ATRA); or (ix) a combination of any one or more of the foregoing therapeutic compounds In some embodiments, the therapeutic compound is selected from ATRA, PLX3397, L-NAME, SD208, Epacadostat, an anti-PD-1 antibody or antigen binding fragment thereof, or a combination thereof.

### Certain Immunoconjugates

Some embodiments of the methods and compositions provided herein include immunoconjugates or antibody-drug conjugates (ADCs) comprising an anti-biotin antibody or antigen binding fragment thereof as provided herein, conjugated to one or more cytotoxic agents, such as chemotherapeutic agents or drugs, growth inhibitory agents, toxins, such as protein toxins, enzymatically active toxins of bacterial, fungal, plant, or animal origin, or fragments thereof, radioactive isotopes.

In some embodiments, an immunoconjugate is an antibody-drug conjugate (ADC) in which an antibody or antigen binding fragment thereof is conjugated to one or more drugs, such as a maytansinoid (see e.g., U.S. Pat. Nos. 5,208,020, 5,416,064 and EP 0 425 235 B1); an auristatin such as monomethyl auristatin drug moieties DE and DF (MMAE and MMAF) (see e.g., U.S. Pat. Nos. 5,635,483, 5,780,588, and 7,498,298); a dolastatin; a calicheamicin or derivative thereof (see e.g., U.S. Pat. Nos. 5,712,374, 5,714,586, 5,739,116, 5,767,285, 5,770,701, 5,770,710, 5,773,001, and 5,877,296; Hinman, L. M., et al., Cancer Res. 53 (1993) 3336-3342; and Lode, H. N., et al., Cancer Res. 58 (1998) 2925-2928); an anthracycline such as daunomycin or doxorubicin (see e.g., Kratz, F., et al., Curr. Med. Chem. 13 (2006) 477-523; Jeffrey, S. C., et al., Bioorg. Med. Chem. Lett. 16 (2006) 358-362; Torgov, M. Y., et al., Bioconjug. Chem. 16 (2005) 717-721; Nagy, A., et al., Proc. Natl. Acad. Sci. USA 97 (2000) 829-834; Dubowchik, G. M., et al., Bioorg. & Med. Chem. Letters 12 (2002) 1529-1532; King, H. D., et al., J. Med. Chem. 45 (20029 4336-4343; and U.S. Pat. No. 6,630,579); methotrexate; vindesine; a taxane such as docetaxel, paclitaxel, larotaxel, tesetaxel, or ortataxel; a trichothecene; or CC1065. All the aforementioned references are expressly incorporated by reference in their entireties.

In some embodiments, an immunoconjugate comprises an antibody or antigen binding fragment thereof conjugated to an enzymatically active toxin or fragment thereof, such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, sapaonaria officinalis inhibitor, gelonin, mitogellin, restrictocin, phenomycin, enomycin, or the tricothecenes.

In some embodiments, an immunoconjugate comprises an antibody or antigen binding fragment thereof conjugated to a radioactive atom to form a radioconjugate. A variety of radioactive isotopes are available for the production of radioconjugates. Examples include At211, I131, I125, Y90, Rel86, Sm153, Bi212, P32, Pb212 or the radioactive isotopes of Lu. When the radioconjugate is used for detection, it may comprise a radioactive atom for scintigraphic studies, for example TC99m or 1123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

In some embodiments, conjugates of an antibody or an antigen binding fragment thereof and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta, E. S., et al., Science 238 (1987) 1098-1104. Carbon-14-labeled 1-isothiocyanatobenzyl-3-methyldiethylene triamine pentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari, R. V., et al., Cancer Res. 52 (1992) 127-131; U.S. Pat. No. 5,208,020) may be used.

In some embodiments, the immunoconjugates or ADCs include such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, or sulfo-SMPB, or SVSB (succinimidyl-(4-vinylsulfone)benzoate), which are commercially available (e.g., from Pierce Biotechnology, Inc., Rockford, Ill., U.S.A).

### Certain Biotinylated Compounds

Some embodiments of the methods and compositions provided herein include use of biotinylated compounds, such as biotin conjugated to a molecule, such as conjugated biotin. In some embodiments, the biotin is conjugated to the molecule via a linker. Examples of molecules includes fluorescein, a fluorescein derivative, folic acid, folate, a folate derivative, dinitrophenol (DNP), a phospholipid ether (PLE), polyethylene glycol (PEG), cholesterol, a lipid, a protein, a Bite, or an aptamer. Examples of linkers include phospholipid ether (PLE), polyethylene glycol (PEG), and polypeptides.

In some embodiments, the linker has a length such that the conjugated biotin has a mass in a range from about 100 Da to about 5000 Da, from about 200 Da to about 4000 Da. from about 300 Da to about 3000 Da, from about 400 Da to about 2000 Da, from about 500 Da to about 1000 Da, from about 500 Da to about 1500 Da, from about 1000 Da to about 2000 Da, from about 2000 Da to about 2500 Da, from about 2500 Da to about 3000 Da, from about 3000 Da to about 3500 Da, from about 3500 Da to about 4000 Da, from about 4500 Da to about 5000 Da, or from about 5000 Da to about 6000 Da. For example, the linker can include a polypeptide such that the conjugated biotin has a mass in a range from about 100 Da to about 5000 Da, from about 200 Da to about 4000 Da. from about 300 Da to about 3000 Da, from about 400 Da to about 2000 Da, from about 500 Da to about 1000 Da in a range from about 100 Da to about 5000 Da, from about 200 Da to about 4000 Da. from about 300 Da to about 3000 Da, from about 400 Da to about 2000 Da, from about 500 Da to about 1000 Da. In some embodiments, the conjugated biotin comprises folate, a polypeptide linker, and a mass in a range from about 400 Da to about 2000 Da, from about 500 Da to about 1000 Da, such as 700 Da.

In some embodiments, the biotin conjugated to the molecule is capable of specifically binding the anti-conjugated biotin antibodies or antigen binding fragments thereof provided herein.

In some embodiments, the molecule is capable of specifically binding a ligand and/or cell, and/or preferentially binding a ligand and/or a cell. For example, the molecule can include a folate, folic acid or derivatives thereof. In some embodiments, the folate can be folic acid, a folic acid analog, or another folate receptor-binding molecule. In various embodiments, analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate).

In some embodiments, the molecule can include a lipid of phospholipid capable of integrating into a plasma membrane of a target cell. In some embodiments, the molecule can include an antibody of antigen binding fragment thereof capable of binding a target antigen. Examples of target antigens include cancer cell antigens, such as EGFR or CD19. In some embodiments, the molecule lacks a capability of specifically binding a ligand and/or cell. For example, the molecule can include a detectable marker, such as fluorescein or a fluorescein derivative.

In some embodiments, the biotin can be covalently joined to the molecule via a linker. In some embodiments, the linker can comprise polyethylene glycol (PEG), polyproline, a hydrophilic amino acid, a sugar, an unnatural peptidoglycan, a polyvinylpyrrolidone, pluronic F-127, or a combination thereof.

Certain aspects disclosed in the following are useful with certain embodiments provided herein: U.S. Pat. Nos. 11,311,576; 11,649,288; US 2020/0354477, which are each incorporated by reference herein in its entirety.

### EXAMPLES

### Example 1—Generating a CAR Specific to Conjugated Biotin

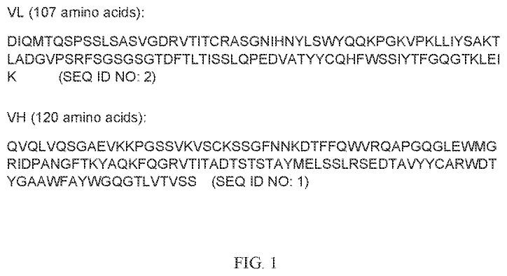

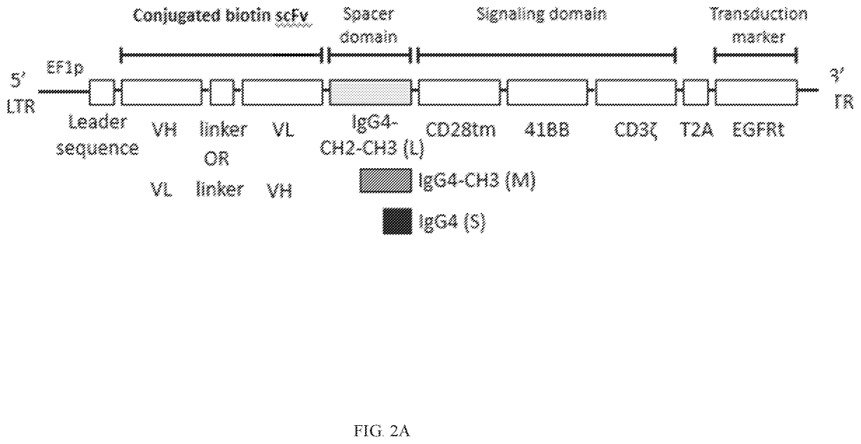

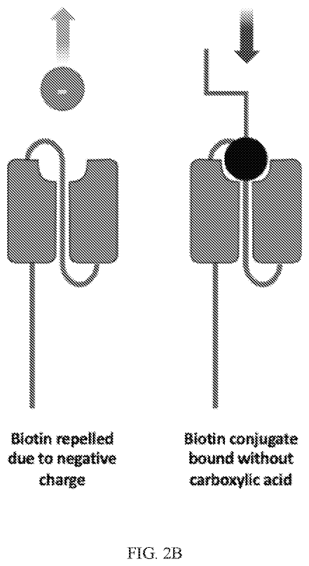

A series of constructs for conjugated-biotin-targeting CARs were generated as shown in FIG. 2. These constructs included a leader/signal sequence, an EGFRt transduction marker, T2A, a signaling domain, a spacer domain, and a scFv against conjugated biotin. The scFv region included VH and VL domain in either a VH/VL or VL/VH orientation, with sequences as shown in FIG. 1 (SEQ ID NOs: 1-2). Different constructs were also generated with either an IgG4 spacer domain (“S”), an IgG4-CH3 spacer domain (“M”), or an IgG4-CH2-CH3 spacer domain (“L”) (SEQ ID NOs: 15-20).

### Example 2—Expressing Conjugated-Biotin-Targeting CARs in Cell Lines

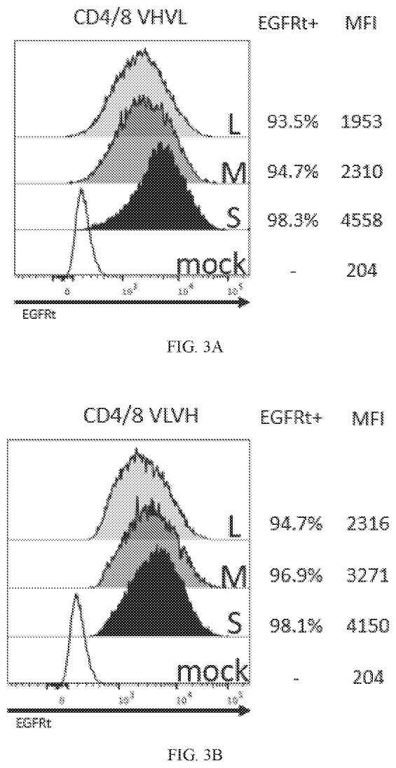

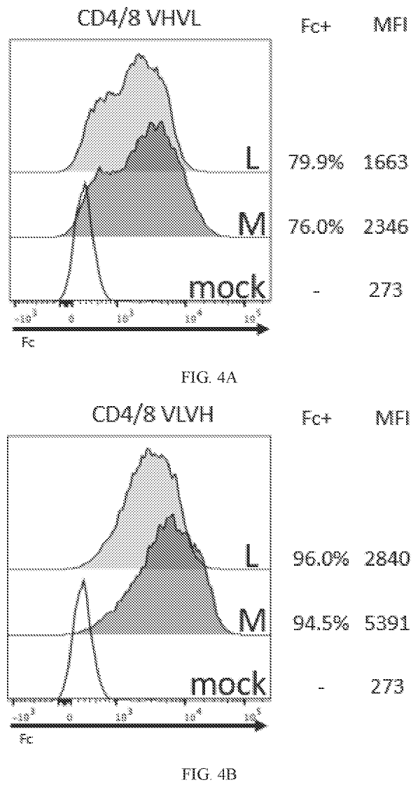

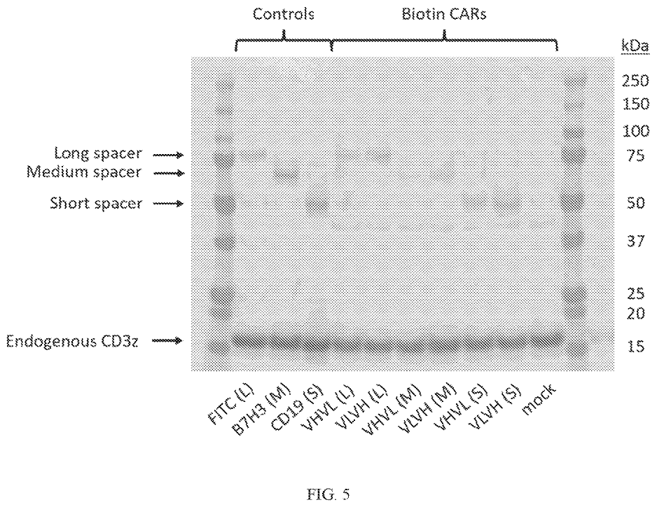

Once generated, the constructs were transduced into a mixed population of CD4/CD8 cells. On day 9 (S1D9), cells were analyzed via flow cytometry for CAR expression using an anti-EGFR antibody, as shown in FIGS. 3A-3B, and an anti-Fc antibody, as shown in FIGS. 4A-4B. Cells were also screened for CD3-zeta expression using a western blot on day 15 (S1R1D15) (FIG. 5).

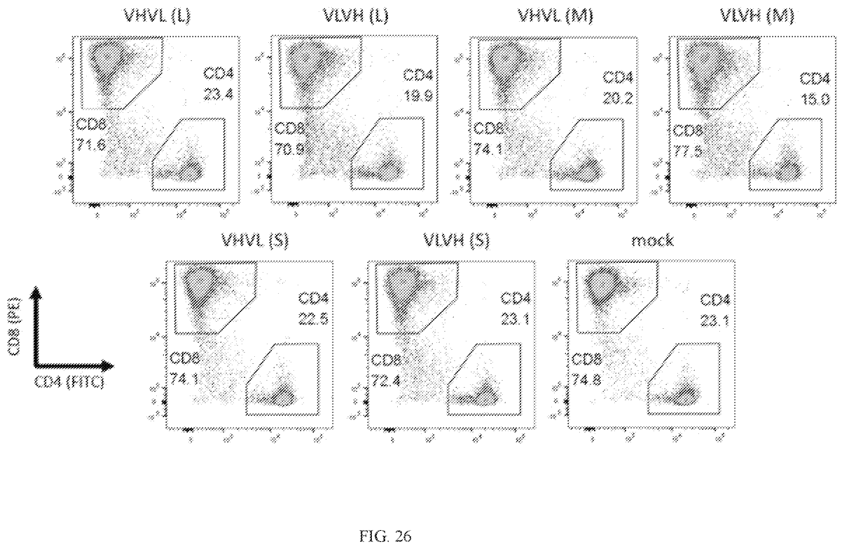

CD4/8 splits of the CAR T cells were assessed via flow cytometry. FIG. 26 depicts the CD4/8 distributions for VHVL “L,” or long spacer cells, VLVH L cells, VHVL “M”, or medium spacer cells, VLVH M cells, VHVL “S”, or short spacer cells, VLVH S cells, and mock cells. For each CAR T cell type, there were more CD8-expressing cells than CD4-expressing cells, although both were strongly present.

### Example 3—Binding of CARs to Conjugated Biotin

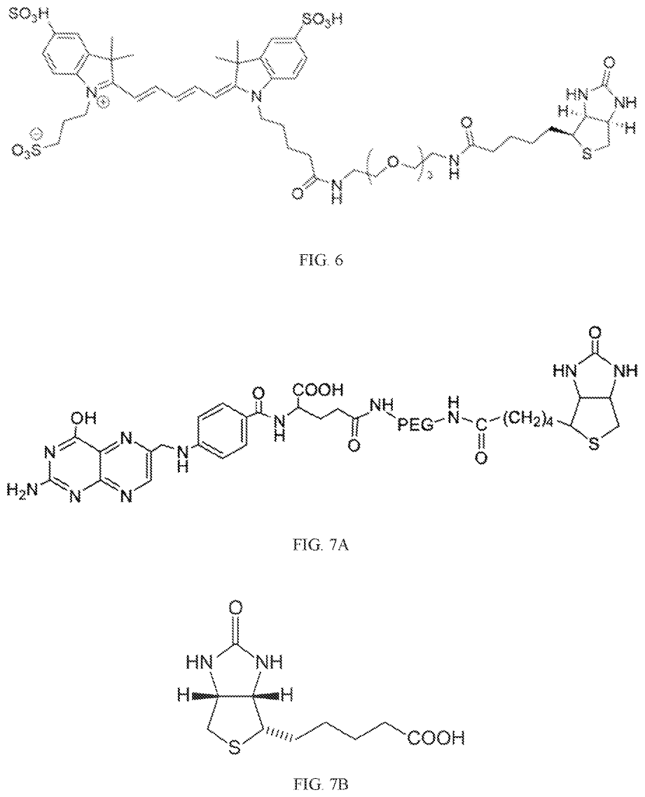

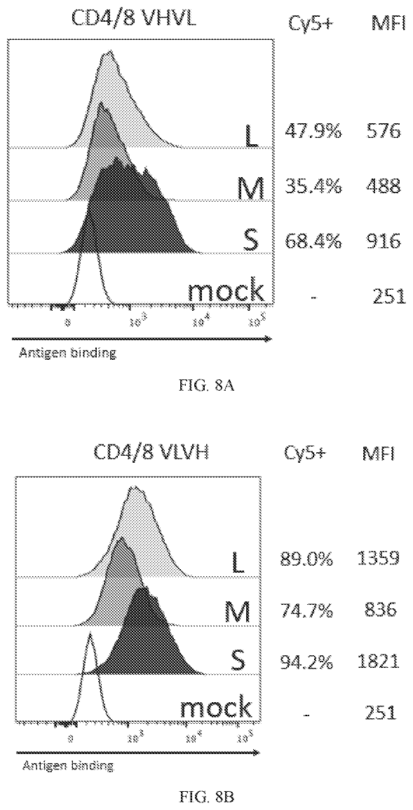

Various biotin derivatives were tested for binding by the conjugated-biotin-targeting CAR cell lines. Fluorescently labeled biotin (FIG. 6) was used to screen the CAR T cell lines for binding to conjugated biotin (FIGS. 8A and 8B). For constructs including VL-linker-VH sequences, antigen binding was higher than for constructs with VH-linker-VL sequences for each respective spacer length. In constructs with VH-linker-VL sequences, the construct with the S spacer had the highest binding to antigen, although all constructs showed higher binding than mock cells (FIG. 8A). In constructs with VL-linker-VH sequences, the constructs with the L or S spacer had the highest binding to antigen, although all constructs showed higher binding than mock cells (FIG. 8B).

### Example 4—Cell Activation Upon CARs Binding to Intermediate-Labeled Cells

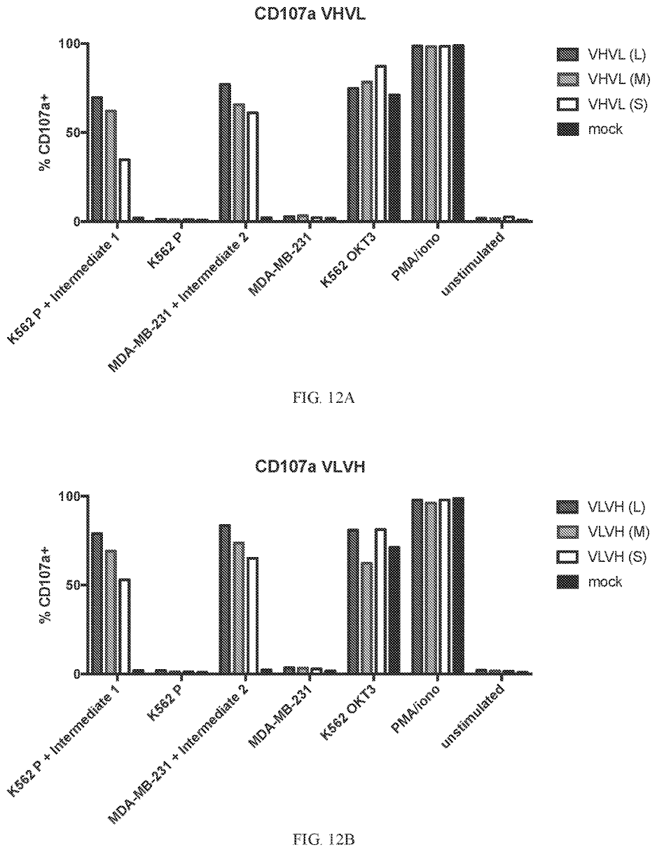

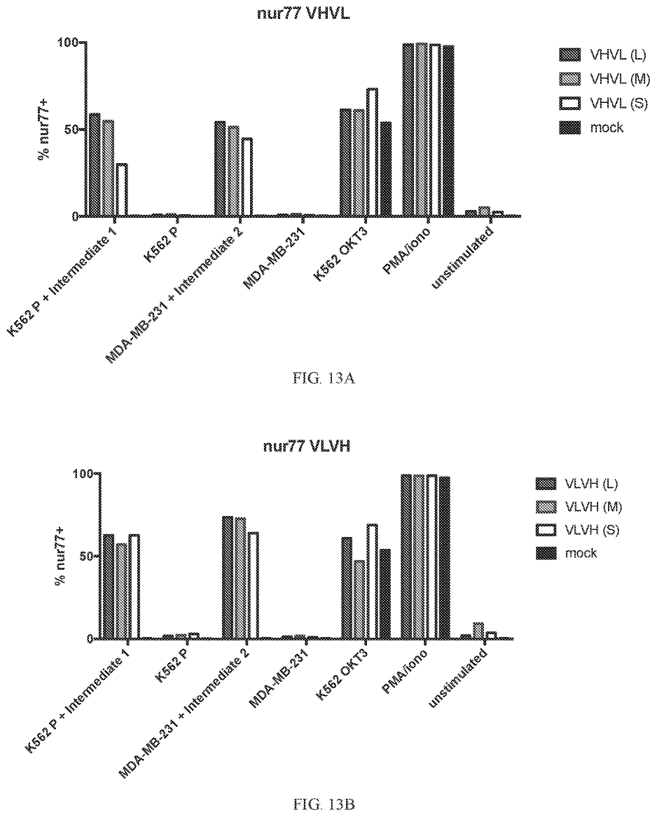

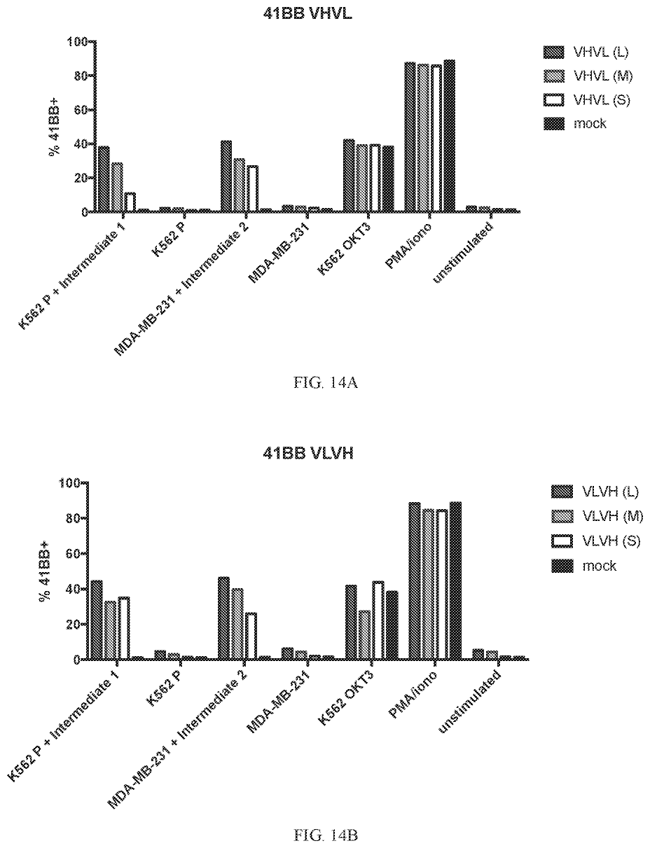

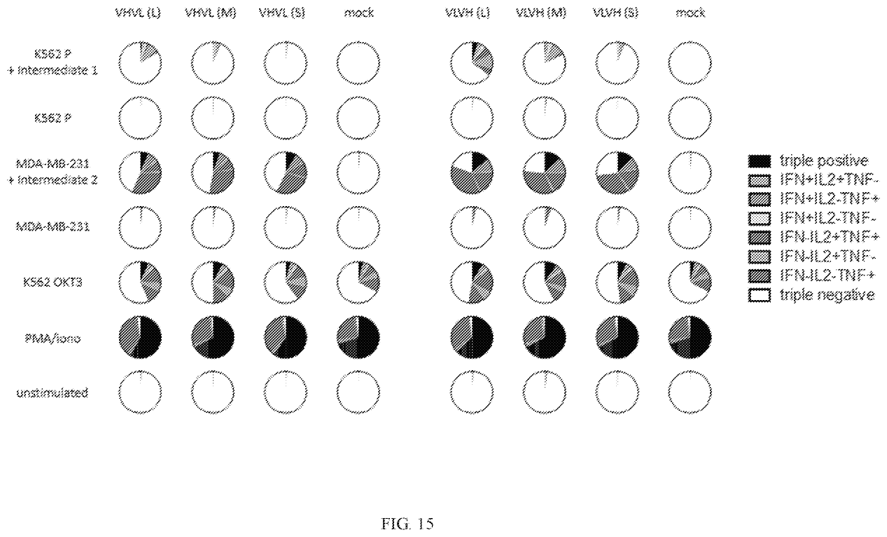

Cells were screened for the expression of activation markers upon exposure to conjugated biotin-labeled target cells. CD4/8+ conjugated-biotin-targeting CAR T cells were co-incubated with K562 P, MDA-MB-231, K562 OKT3 target cells, or PMA/ionomycin for 4 hours at 37° C. Target cells were either unstained, or labeled with FSL-biotin (K562 P cells) (“intermediate 1”) or biotin-folate (MDA-MB-231 cells) (“intermediate 2”). Activation markers CD107a, nur77, and 4-1BB were upregulated in CD8+ T cells when stimulated with intermediate-labelled cells but not unlabeled cells (FIGS. 12A-12B, 13A-13B, and 14A-14B). The cells were then assessed for production of the intracellular cytokines IFN, 112, and TNF (FIG. 15). Similar to the trend with activation marker production, intracellular cytokines increased in CD4+ T cells when stimulated with intermediate-labelled cells, but not unlabeled cells. These data indicated that the anti-biotin CAR T cells only produced cytokines when stimulated with positive controls PMA/ionomycin and K562 OKT3 cells or intermediate-labelled cells.

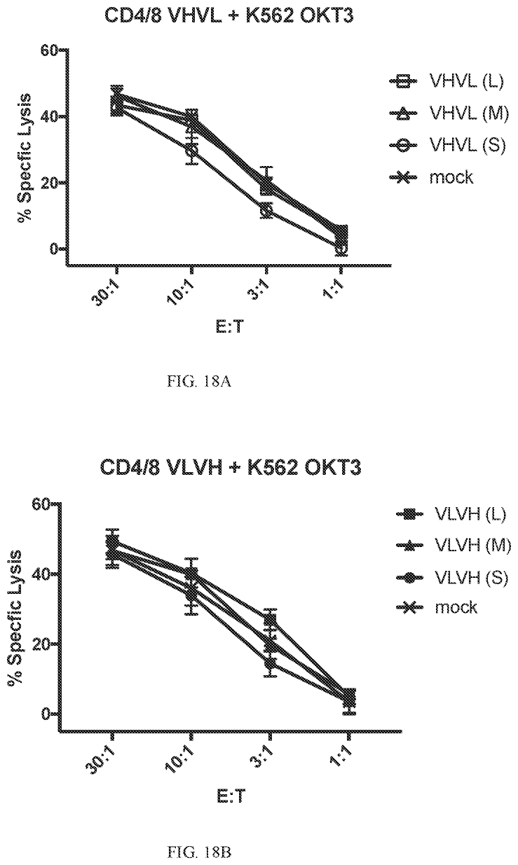

Target cells were then incubated with various effector:target (E:T) ratios of mixed CD4/8 anti-biotin CAR T cells for 4 hours at 37° C. Specific lysis of target cells was measured by chromium released from chromium-loaded target cells (chromium loaded overnight at 37° C.). The ratio of effector cells to target cells was either 30:1, 10:1, 3:1, or 1:1. In intermediate-labeled K562 cells, specific lysis was higher with the L and M constructs compared to the S construct for both VH-linker-VL and VL-linker-VH, although all three had higher lysis than mock cells (FIGS. 17A-17B). This was an experimental group and showed the ability of the anti-biotin CAR T cells to specifically lyse cells labelled with conjugated biotin. VLVH orientation and long/medium spacers qualitatively performed better than VHVL and short spacer CARs.

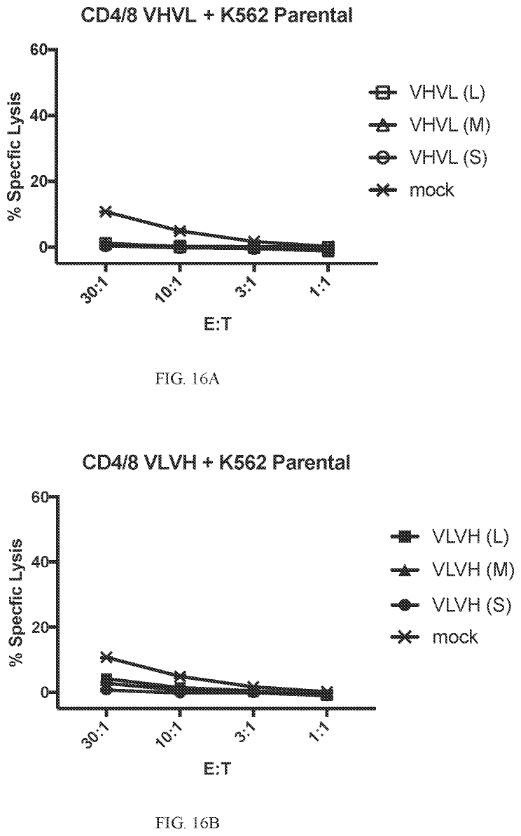

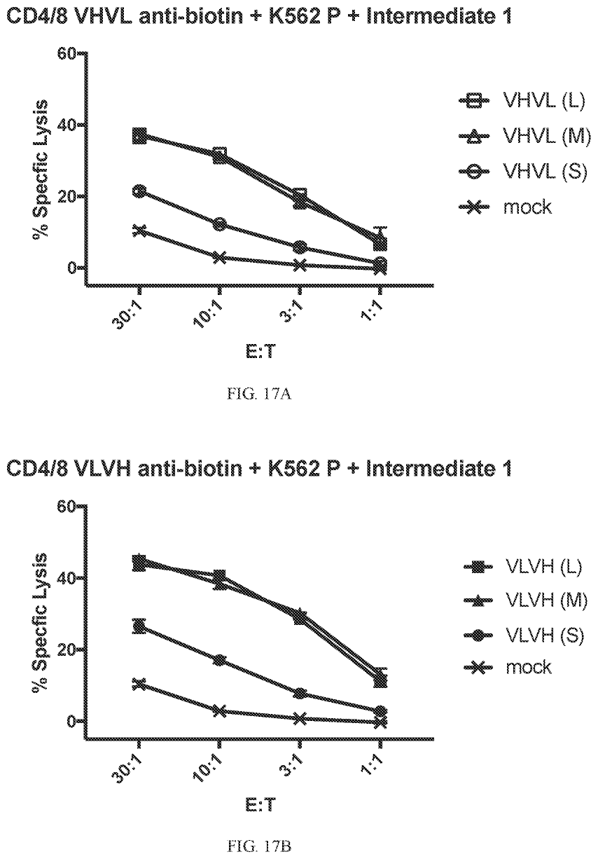

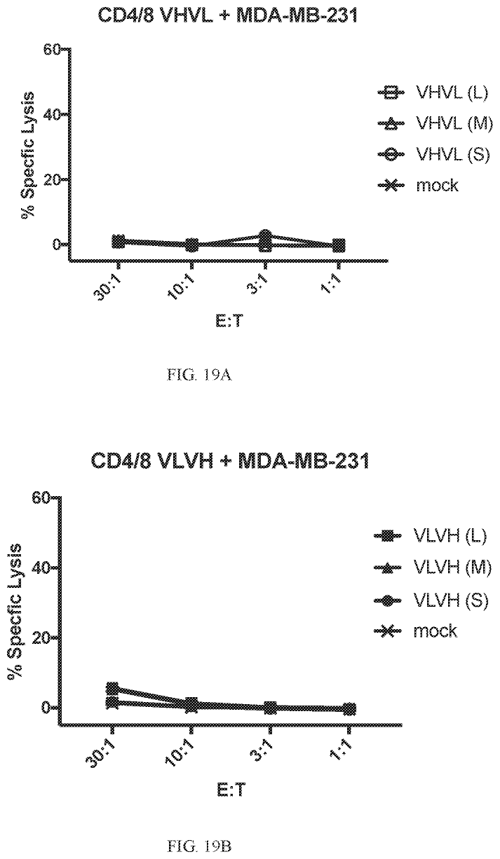

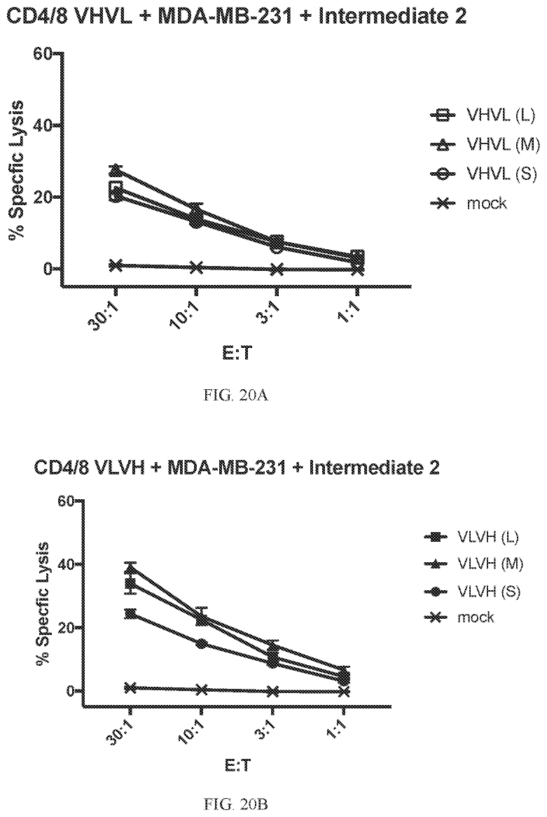

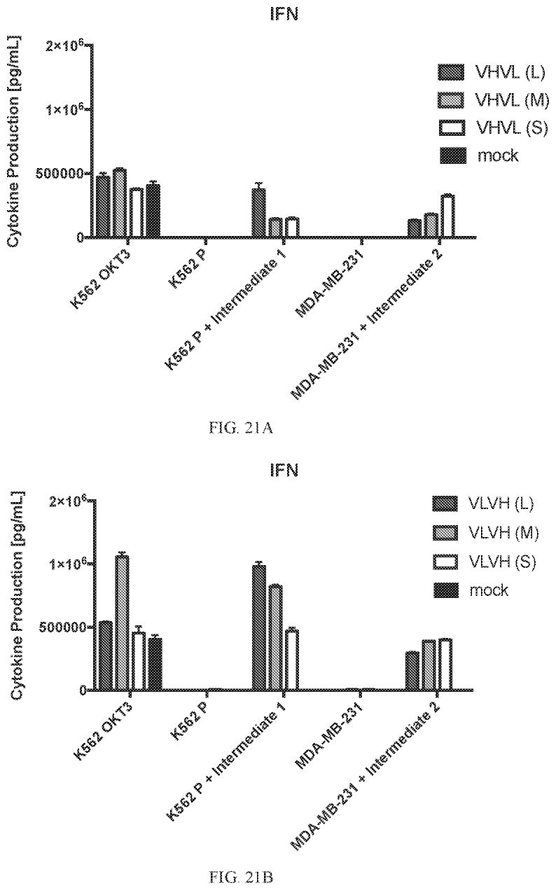

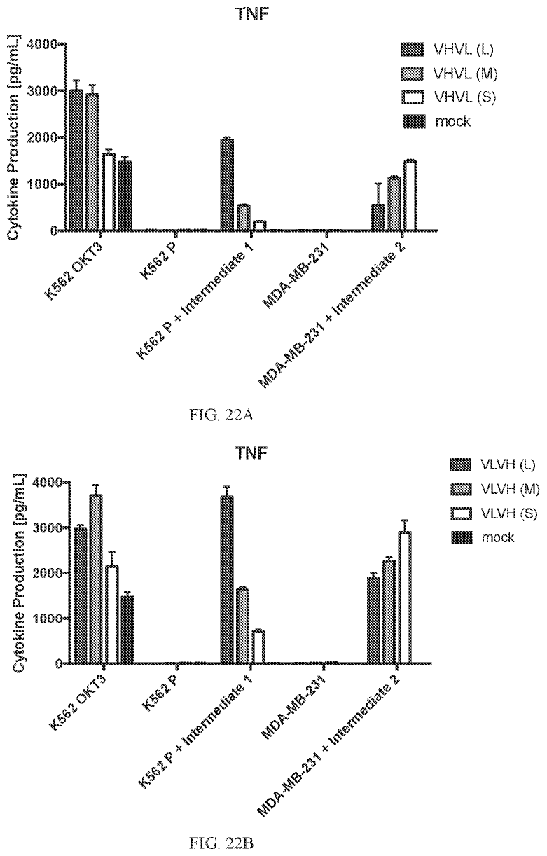

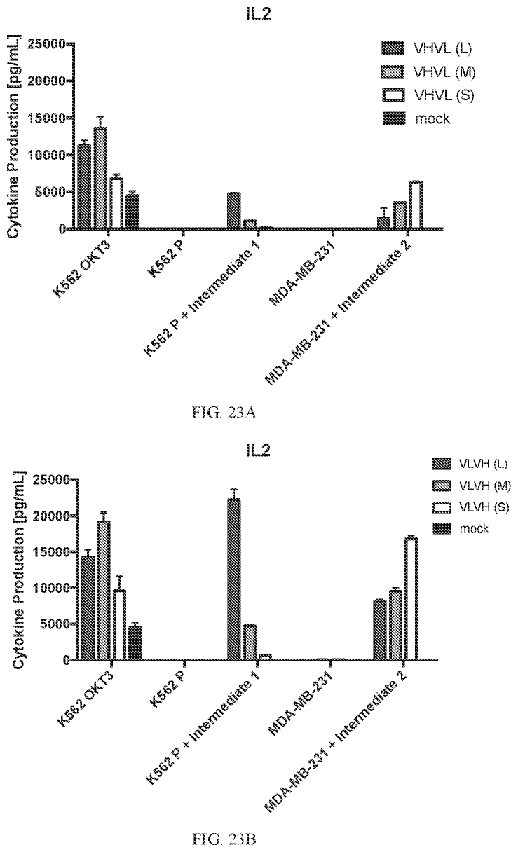

This trend carried to intermediate-labelled MDA-MB-231 target cells for the VL-linker-VH construct, yet the S construct was equally efficient as the L and M construct for the VH-linker-VL sequences (FIGS. 20A and 20B). This was an experimental group and showed the ability of the anti-biotin CAR T cells to specifically lyse cells labelled with conjugated biotin. VLVH orientation and long/medium spacers qualitatively performed better than VHVL and short spacer CARs. Without intermediate-labelled cells, no significant lysis was observed across all cell types (FIGS. 16A-16B and 19A-19B).

Mixed CD4/8 anti-biotin CAR T cells were incubated with target cells for 24 hours at 37° C. Extracellular IFN, IL2, and TNF was then measured (FIGS. 21A-21B, 22A-22B, and 23A-23B). The efficacy of each CAR construct for releasing extracellular cytokines varied by cell type but were each consistently more efficacious than mock T cells.

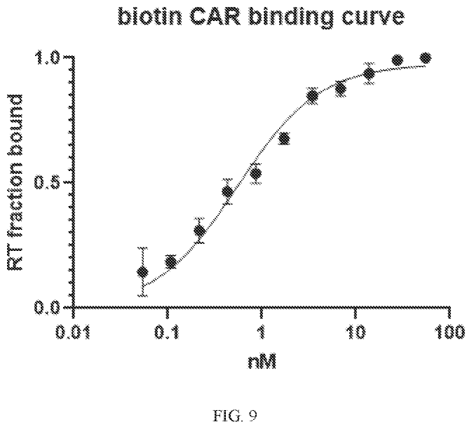

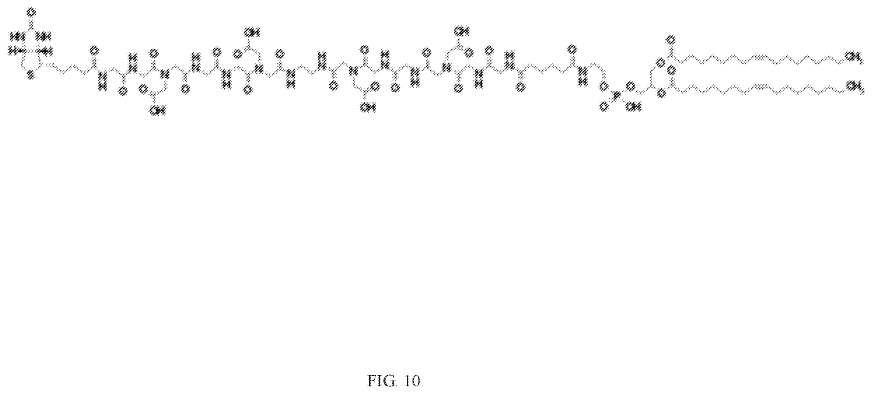

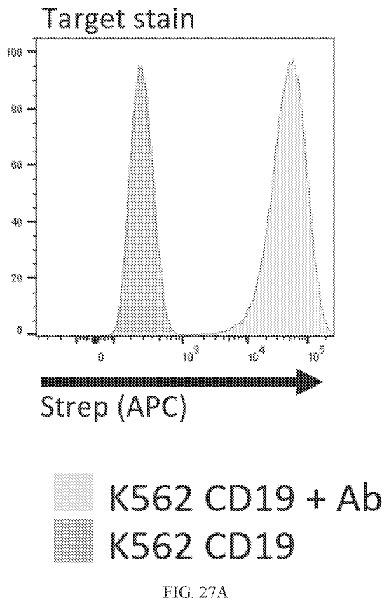

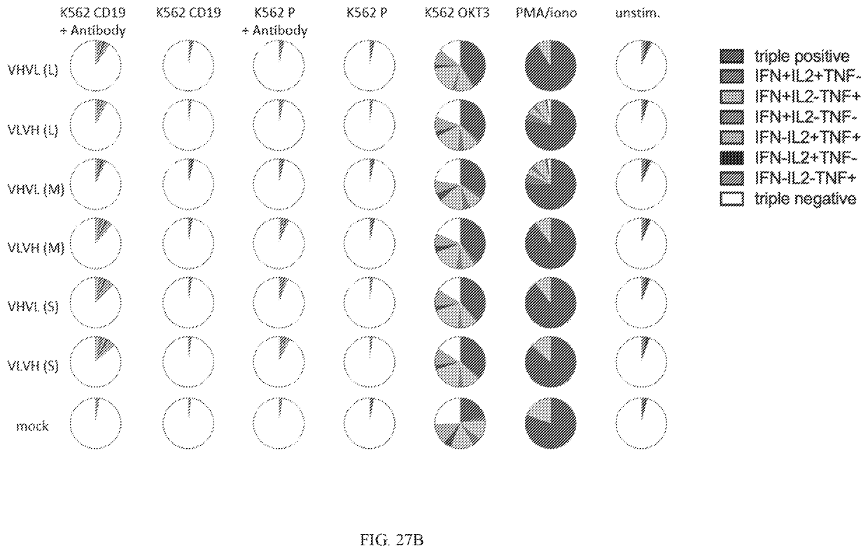

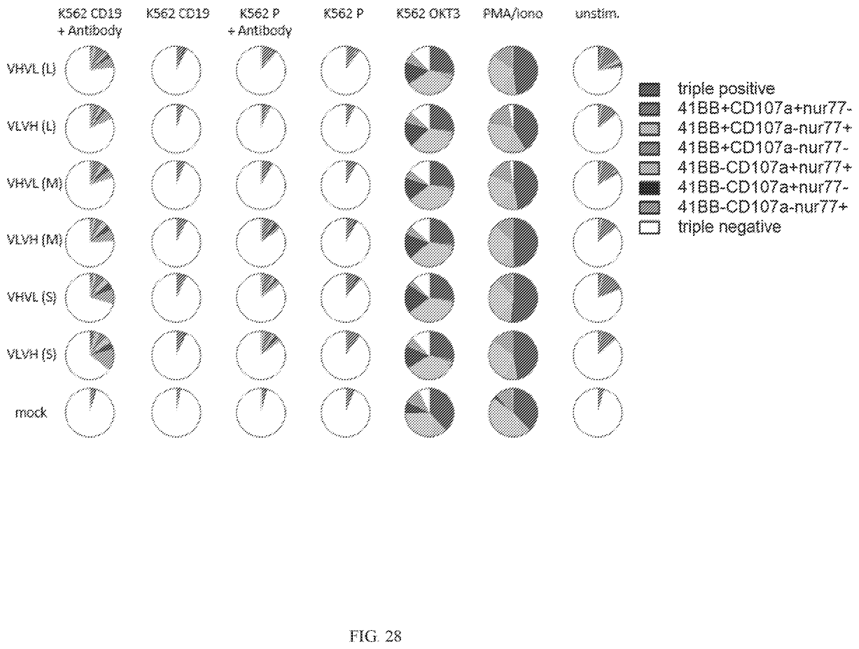

Intermediates 1 and 2 (FIGS. 9 and 10) were holistically the most successful intermediates at activating anti-conjugated biotin CAR T cells, but an additional intermediate was also used to label targets. Here, anti-conjugated biotin CAR T cells were co-incubated with K562 P, K562 CD19, K562 OKT3 target cells, or PMA/ionomycin for 4 hours at 37° C. Target cells were either unstained or labeled with a biotinylated anti-CD19 antibody (“+Antibody”, from Biolegend® Cat. 302203). Activation markers CD107a, nur77, and 41BB were upregulated in CD8+ T cells when stimulated with intermediate-labelled cells but not unlabeled cells (FIG. 28). Excluding K562 OKT3 and PMA/ionomycin positive controls, the greatest upregulation in activation markers was observed when CAR T cells were co-incubated with K562 CD19+Antibody targets, although there was some low level of activation observed in the K562 P+Antibody negative control group. The cells were then assessed for production of the intracellular cytokines IFN, IL2, and TNF (FIG. 27B). Similar to the trend with activation marker production, intracellular cytokines increased in CD4+ T cells when stimulated with intermediate-labelled cells, but not unlabeled cells. These data indicated that the anti-biotin CAR T cells only produce cytokines when stimulated with positive controls PMA/ionomycin and K562 OKT3 cells or intermediate-labelled cells. Furthermore, there was no observed cytokine production from negative control groups.

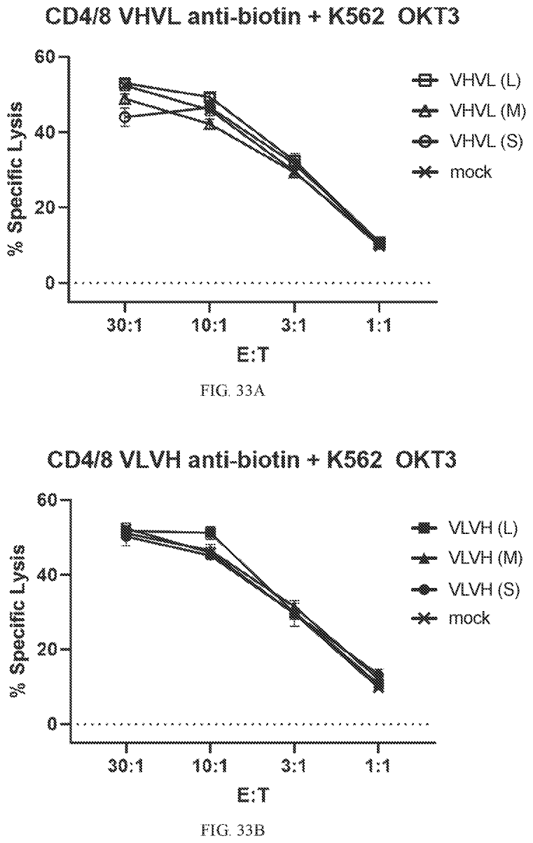

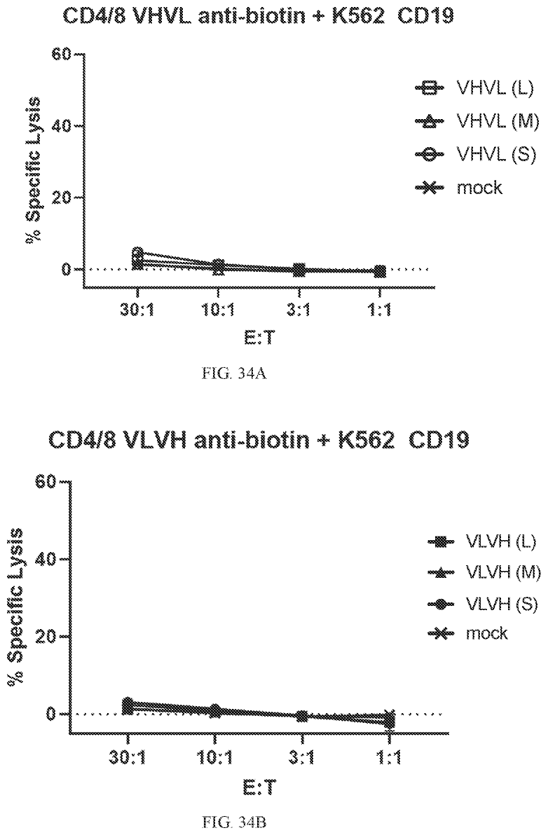

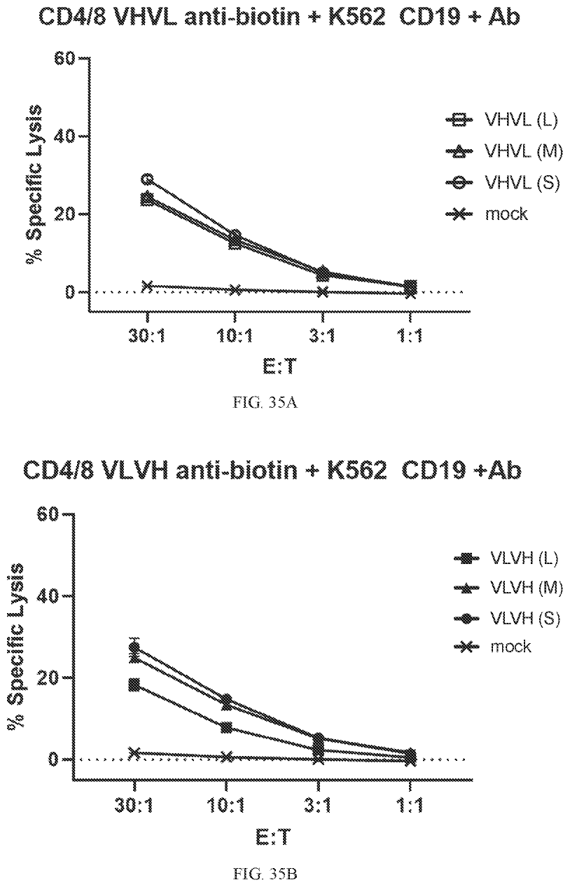

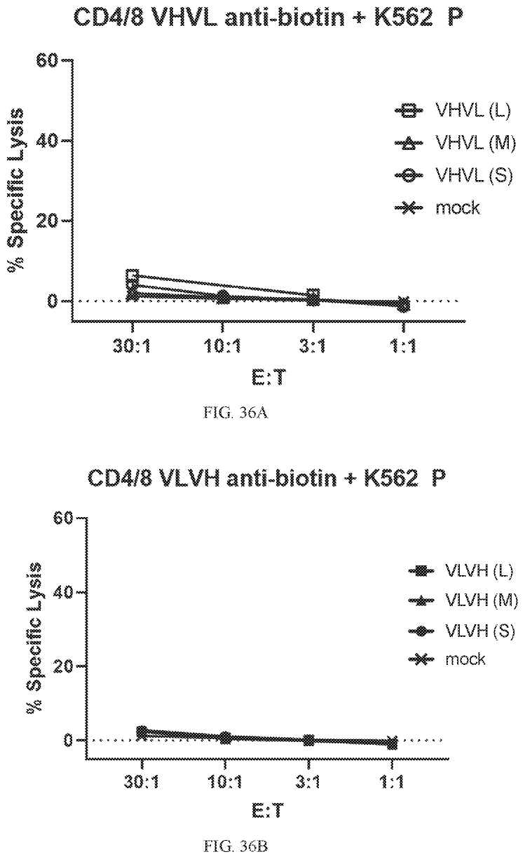

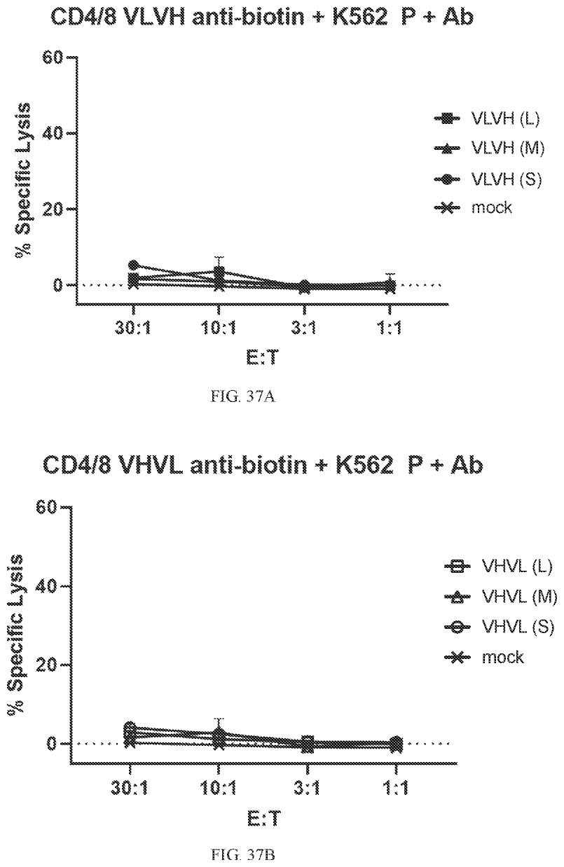

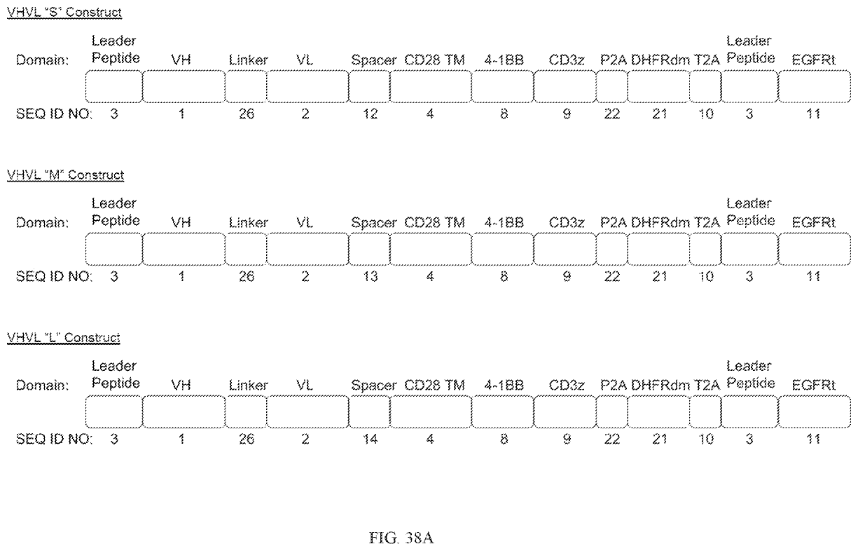

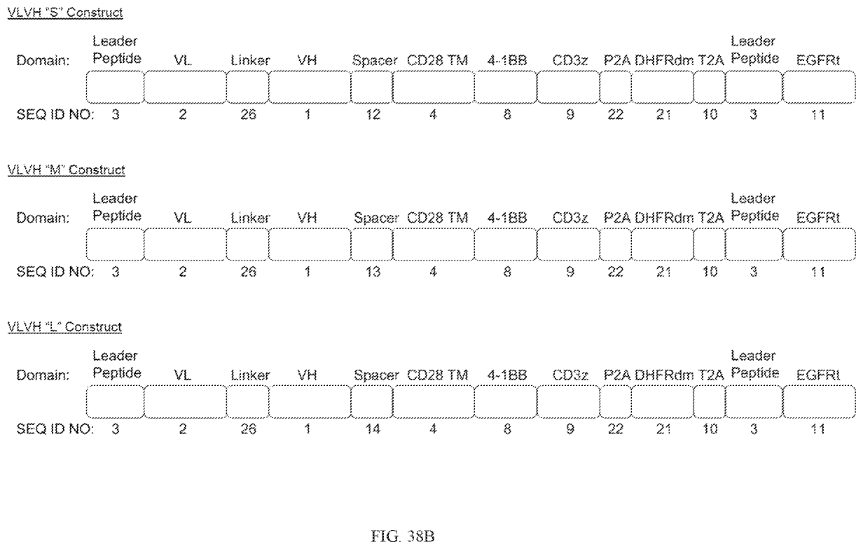

Target cells were then incubated with various Effector:Target (E:T) ratios of mixed CD4/8 anti-biotin CAR T cells for 4 hours at 37° C. Specific lysis of target cells was measured by chromium released from chromium-loaded target cells (chromium loaded overnight at 37° C.). The ratio of effector cells to target cells was either 30:1, 10:1, 3:1, or 1:1. These data demonstrated specific lysis of exclusively K562 OKT3 cells and K562 CD19+ antibody cells, therefore further demonstrating the antigen specificity of the anti-conjugated biotin CAR T cells (FIGS. 33A-37B). In antibody-labeled K562 CD19 cells, specific lysis was higher with all anti-conjugated biotin CARs than mock cells (FIGS. 35A-35B).

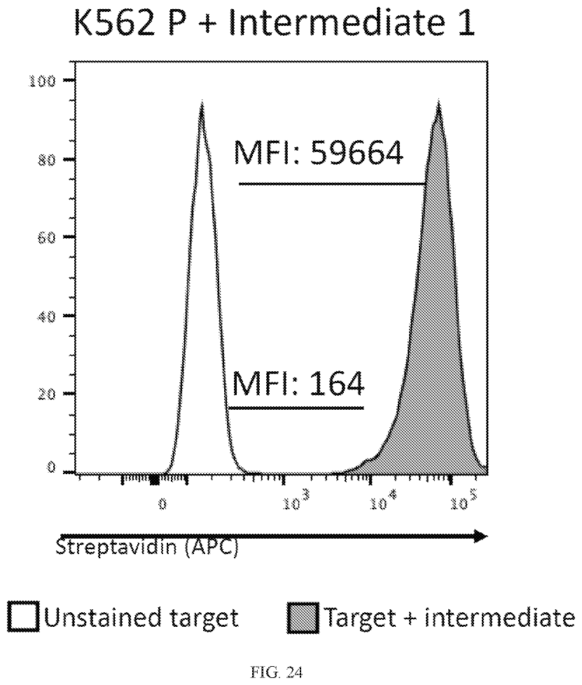

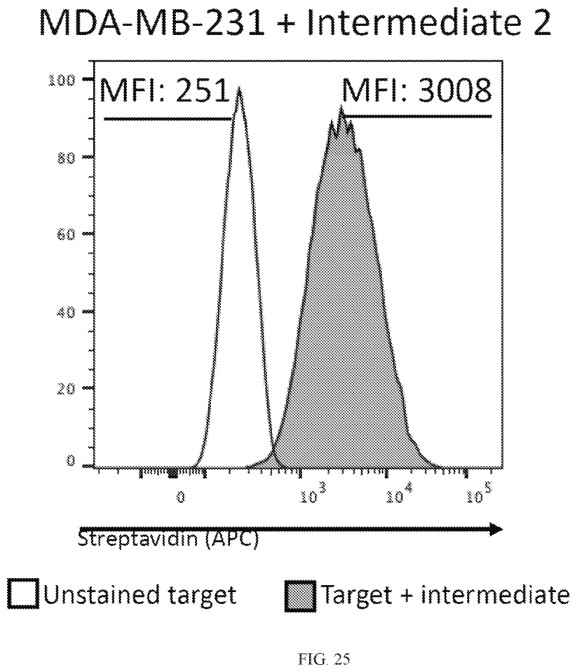

As a control, streptavidin was used to analyze biotin expression in target cells for these functional assays. Cells labeled with FSL-biotin (FIG. 24), biotin folate (FIG. 25), and biotinylated anti-CD19 antibody (FIG. 27A) expressed biotin at much higher levels than unstained cells.

### Example 5—Specificity of CARs for Conjugated Versions of Biotin

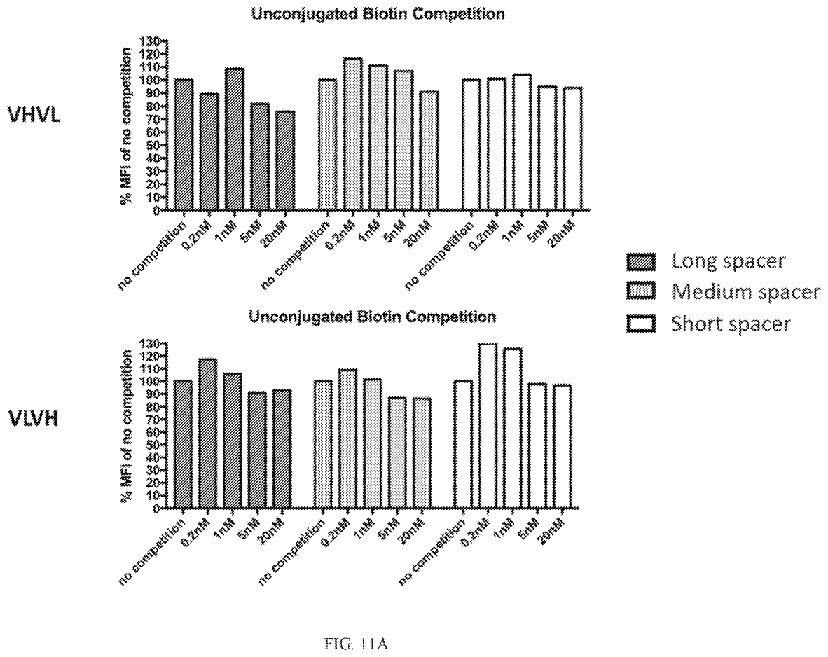

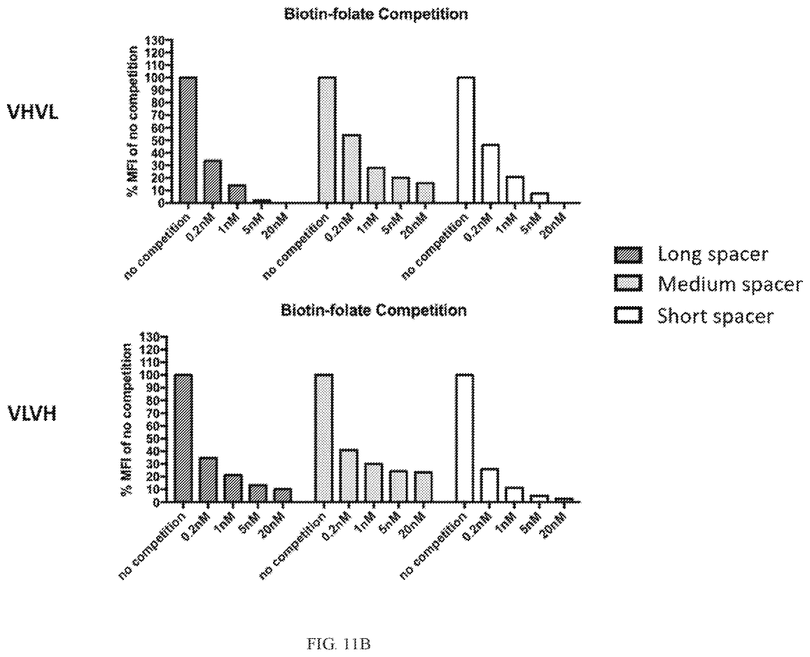

To demonstrate specificity for conjugated biotin, a series of competition assays were performed and analyzed by flow cytometry. First, cells expressing various CARs were incubated with 440 nM of biotin-PEG-Cy5 (FIG. 6) for 30 minutes at room temperature. Concurrently, they were exposed to 0 nM (no competition), 0.2 nM, 1 nM, 5 nM, or 20 nM of either unconjugated biotin or a non-fluorescent conjugated biotin (FIG. 7A). Cells exposed to competition with unconjugated biotin were not negatively impacted in their ability to bind biotin conjugated to fluorescent Cy5 (FIG. 11A). In contrast, cells exposed to competition with a non-fluorescent conjugated biotin (FIG. 7A) demonstrated reduced binding to biotin conjugated to fluorescent Cy5 in a concentration-dependent manner (FIG. 11B).

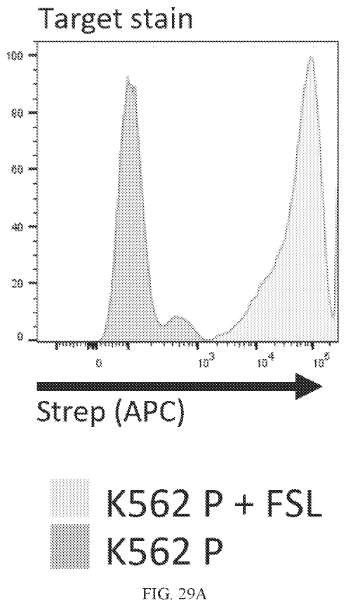

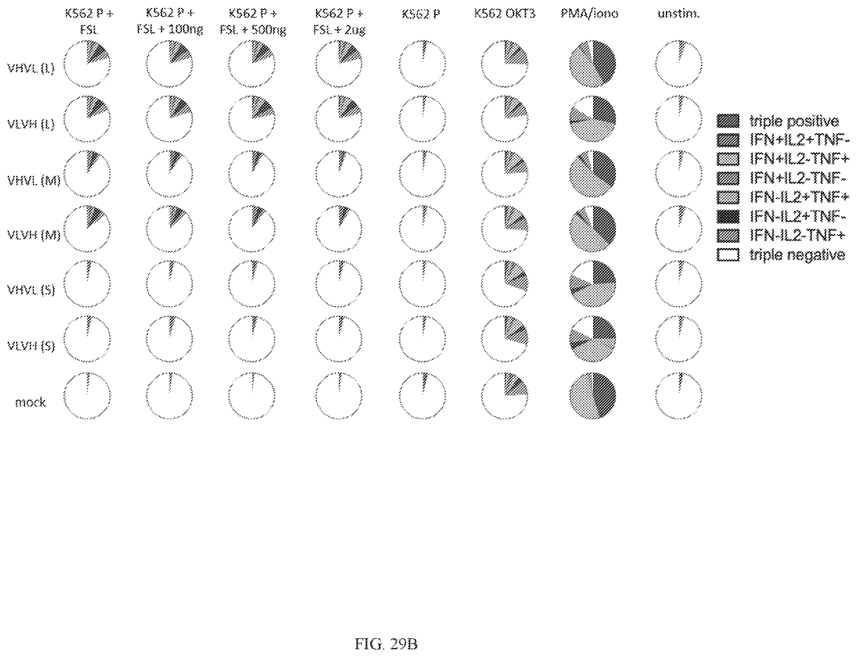

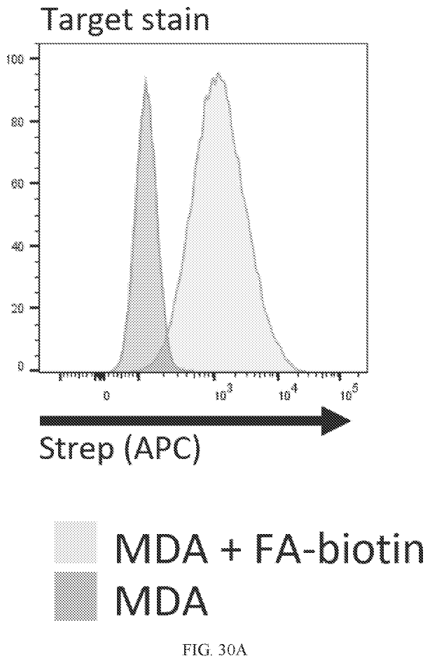

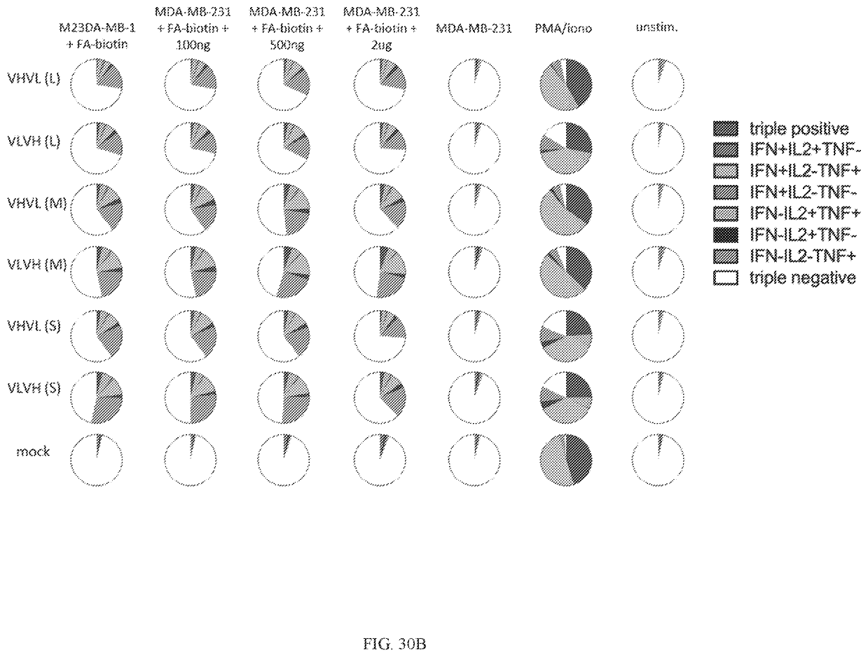

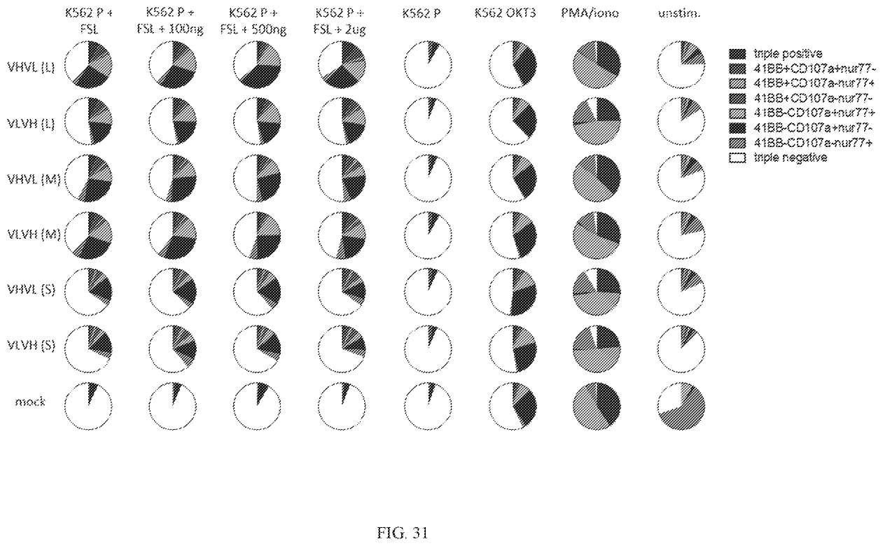

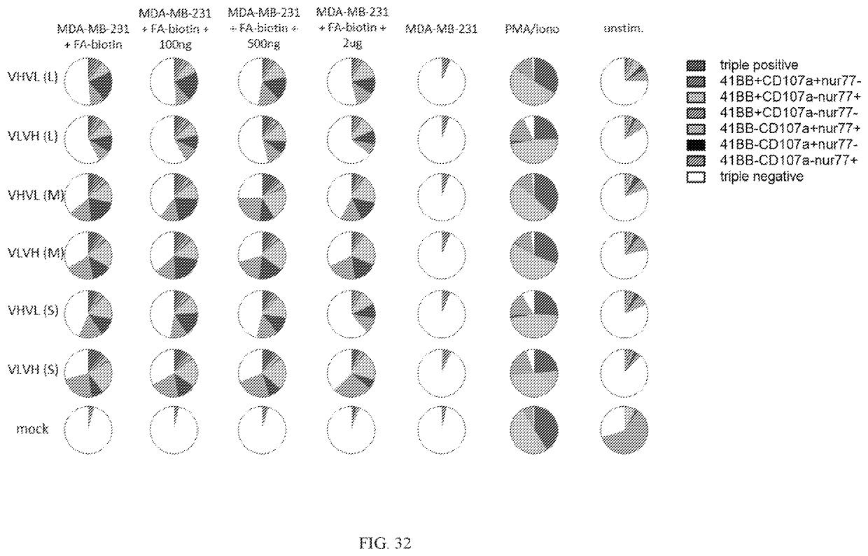

Next, CD4/8+ conjugated-biotin-targeting CAR T cells were co-incubated with K562 P, MDA-MB-231, K562 OKT3 target cells, or PMA/ionomycin for 4 hours at 37° C. Target cells were either unstained or labeled with FSL-biotin (K562 cells) (“intermediate 1”) or biotin-folate (MDA-MB-231 cells) (“intermediate 2”). Additionally, unconjugated biotin was added to co-incubating cells for the entire 4-hour co-incubation to determine whether unconjugated biotin can block anti-conjugated biotin binding to target cells. Unconjugated biotin was added at 4 different doses: 0 ng (no competition), 100 ng, 500 ng, and 2 ug. Activation markers CD107a, nur77, and 41BB were upregulated in CD8+ T cells when stimulated with intermediate-labelled cells but not unlabeled cells, and the unconjugated biotin dose had no impact on the activation profiles of the CAR T cells (FIGS. 31 and 32). The cells were then assessed for production of the intracellular cytokines IFN, IL2, and TNF. Similar to the trend with activation marker production, intracellular cytokines increased in CD4+ T cells when stimulated with intermediate-labelled cells, but not unlabeled cells, and unconjugated biotin had no impact on the activation profiles of the CAR T cells (FIGS. 29B and 30B). These data indicated that the function of anti-biotin CAR T cells was not impacted by environmental biotin, even at high concentrations.

The anti-tumor efficacy of the cells have proven to be highly sensitive to the bispecific chemical intermediate used (e.g. biotinylated antibodies vs. biotin-folate vs. biotinylated lipids). Furthermore, the in vitro data showed high anti-tumor functionality with the biotin-folate, a biotinylated lipid, and a biotinylated antibody.

### Example 6—Development of New Universal CARs

Chimeric antigen receptor (CAR) T cells have enabled substantial progress in the treatment of hematological malignancies,^{1,2} resulting in six FDA-approved CAR T cell treatments to date. Although many clinical trials have accomplished complete remission rates of 70-90% using anti-CD19 CAR T cells,^{1,3-5} relapse rates between 30% and 60% are not uncommon.⁶ Due to the selective pressure imposed by CAR T cells on the targeted antigen, malignant cells with certain mutations are resistant to the therapy and tend to thrive.⁷⁻⁹ As a result, it has been documented that 10-20% of responding patients ultimately relapse with antigen-negative disease.¹⁰ To prevent antigen negative relapse, future CAR T cells should account for diverse antigen profiles in tumors. This would be especially useful as CAR T cells are increasingly used in pre-clinical and clinical applications for solid tumors, which are notoriously more heterogenous and complex than hematological cancers.^{11,12} Another set of limitations to CAR T cell therapy are toxicities, such as cytokine release syndrome (CRS), a systemic, high-level immune reaction,¹³ neurological toxicities,¹⁴ and on-target/off-tumor recognition.^{15,16} Tumor-specific antigens are rare, so most therapies target tumor associated antigens (TAAs). Although highly upregulated on malignant cells, TAAs are also found on select healthy tissues, thereby making those tissues vulnerable to direct damage by CAR T cells. Currently, toxicities can be managed with corticosteroids and tocilizumab,¹⁷ but substantial effort is underway to improve our ability to regulate CAR T cell side effects. Due to the prevalence, unpredictability, and severity of these toxicities, enhancements to clinician control over CAR T cell activity are needed.

The control, modularity, and multivalent targeting required of next generation CAR T cell therapies are embodied in a new class of receptors: universal CARs.¹⁸ Universal CARs do not have specificity for a TAA or tumor-specific antigen. Rather, they bind a non-native epitope conjugated to a tumor targeting moiety. These targeting molecules, which effectively form bridges between CAR and target, are termed bifunctional intermediate adaptor molecules and can include small molecules, antibodies, aptamers, peptides, or bispecific T cell engagers (BiTEs).¹⁸ The highly modular nature of these systems is perfectly situated to address heterogenous tumor antigen profiles. By retaining a constant CAR epitope and altering tumor targeting domains, intermediate adaptors enable a single CAR to target multiple TAAs simultaneously. The short half-life of the intermediates also allows for highly regulated CAR T cell function and disengagement of cytotoxic function at the onset of severe side-effects. Intermediate dose can be adjusted patient-to-patient and quenching agents can be administered in the case of on-target, off-tumor toxicity to block CAR/intermediate interactions.¹⁹ Due to their promise, several universal receptors have been developed, including leucine zippers in the zipCAR,²⁰ and CARs for 5B9,²¹ a peptide motif derived from La/SS-B,²²⁻²⁴ peptide neo-epitope (PNE),²⁵ a peptide found in yeast transcription facto GCN4,²⁶ and fluorescein isothiocyanate (FITC), a synthetic dye.²⁷

Recently, similar universal receptors have been developed that bind biotin.^{28,29} Rather than utilizing a short chain variable fragment (scFv), the biotin-binding immunoreceptor (BBIR) CAR and monomeric streptavidin 2 (mSA2) receptor take advantage of the strong biotin/streptavidin bond by using an avidin as the antigen binding domain. Although both receptors demonstrated in vitro efficacy, there was no specificity between endogenous biotin and the biotinylated intermediate. Avidins are also known to be immunogenic, so there are concerns about the antigenicity of the immune receptors themselves.³⁰ To account for these limitations, a CAR was developed that binds to a specific biotin-linker conjugate sequence (AdCAR).³¹

As described herein, a CAR that binds any conjugated derivative of biotin, rather than biotin generally or a specific biotin-linker conjugate is provided. Employing a CAR that binds modified versions of biotin but not the molecule itself enabled use of biologically well-tolerated intermediate adaptors while retaining orthogonality to endogenous systems. This selectivity was possible because of the carboxylic acid present on unmodified biotin, which, in most aqueous environments, takes on a negative charge (FIG. 2B). When bound, biotin is positioned in a cluster of negatively charged amino acids within the binding pocket of the receptor. The negative charge of unmodified biotin therefore elicits a charge repulsion with the binding domain, leading to inefficient binding. Conjugated biotin, on the other hand, is well-suited to couple with the receptor, assuming negative charges near the CAR epitope are absent. As described herein, a panel of CARs was created and their specificity confirmed for conjugated derivatives of biotin. The versatility of the system was modeled by employing two different biotinylated intermediate adaptors to label and eliminate different cancer lines in vitro. In addition, a CAR/intermediate adaptor pairs was demonstrated to have maximized in vivo efficacy. Collectively, these data demonstrated a well-tolerated universal CAR system

### Results

### CAR T Cell Characterization and Confirmation of Antigen Specificity

A panel of anti-conjugated biotin CARs was designed with VHVL and VLVH scFv orientations paired with three different spacer domains (FIG. 2A). The spacer domains tested included a “Short” IgG4 hinge domain, a “Medium” IgG4 hinge-CH3 domain, or a “Long” IgG4 hinge-CH2-CH3 domain.³⁶⁻³⁸ Each spacer was fused to a CD28 transmembrane domain (CD28tm) and a 41BB-CD3ζ intracellular signaling domain to form a second generation CAR. Separated by 2A ribosomal skip sequences were a truncated EGFR (EGFRt) expression marker and a double mutant of dihydrofolate reductase (DHFRdm), which enabled methotrexate drug selection.³⁹ All constructs were codon optimized for humans using ThermoFisher Scientific's GeneArt Gene Optimizer and synthesized by ThermoFisher Scientific's GeneArt Gene Synthesis. The resulting products were cloned into lentiviral vectors. Equivalent numbers of primary human CD4+ and CD8+ T cells were transduced and CAR+ populations were selected with administration of 50 nM methotrexate for six days. Although an equivalent number of CD4 and CD8 T cells were transduced, the cell populations skewed CD8 by the end of the initial cell manufacturing protocol (SID9) (FIG. 26). On the same day, cells were stained for EGFRt expression using an Erbitux-APC conjugate. High CAR EGFRt expression was recorded in all cell groups, although the shorter spacers allowed for higher MFIs with this stain (FIGS. 3A, 3B). High CAR expression, especially in the VLVH groups, was corroborated with an anti-Fc antibody, which is capable of directly staining CARs with medium and long spacer domains (FIGS. 4A, 4B). CAR protein, determined by blotting for CD3ζ, was also confirmed via a western blot on CD3C at a later point in this study (FIG. 5).

To demonstrate the ability of our CARs to bind conjugated biotin, the panel of CAR T cells was incubated with 440 nM of a biotin-PEG-Cy5 conjugate (FIG. 6). Flow cytometry was used to analyze Cy5 expression and confirm that each CAR could bind this biotin conjugate (FIG. 8A, 8B). Once antigen binding was confirmed, a binding study was conducted with titrated doses of biotin-PEG-Cy5. The calculated VLVH CAR Kd to this intermediate was 0.553 nM (FIG. 9), which was slightly higher affinity than the anti-fluorescein CAR had to EC17 (a current benchmark system in the universal CAR space).” This calculation suggested that the CARs were high affinity binders, although it was likely that the CAR Kd would be different to each intermediate.

One differentiating factor for this CAR when compared with other biotin-binding receptors previously described^{28,29,31} is that the CAR may bind any biotin derivative but not the unmodified molecule itself (FIG. 2B). To confirm this selective binding to biotin conjugates, the panel of CAR T cells was incubated with 440 nM of a biotin-PEG-Cy5 conjugate, and titrated doses (20 nM, 5 nM, 1 nM, and 0.2 nM) of competition were incubated concurrently with the soluble antigen. Competition included another conjugated biotin, biotin-PEG2k-folate (FIG. 7A), and unmodified biotin (FIG. 7B). Flow cytometry analysis showed a dose-dependent decrease in MFI with the addition of higher biotin-folate doses. However, equivalent doses of unmodified biotin resulted in no decrease in MFI (FIGS. 11A, 11B). These data illustrated the specificity of the CARs for conjugated biotin.

### In Vitro Efficacy of Anti-Biotin CAR T Cells Targeting Different Biotinylated Intermediate Adaptors

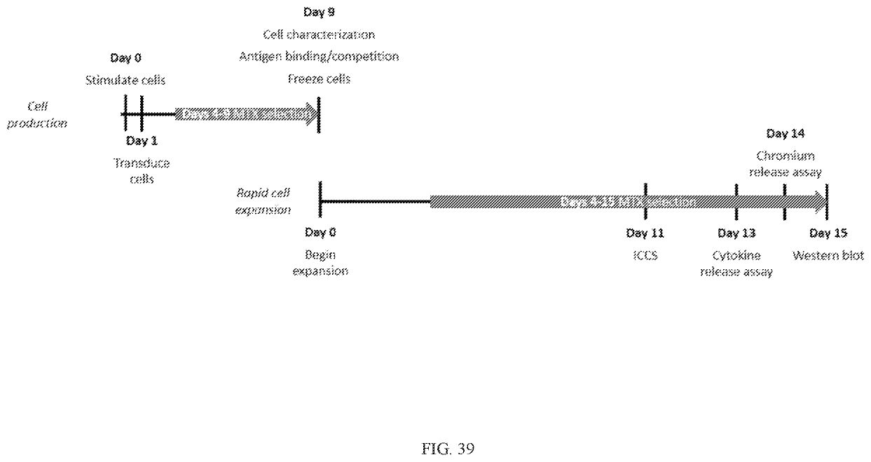

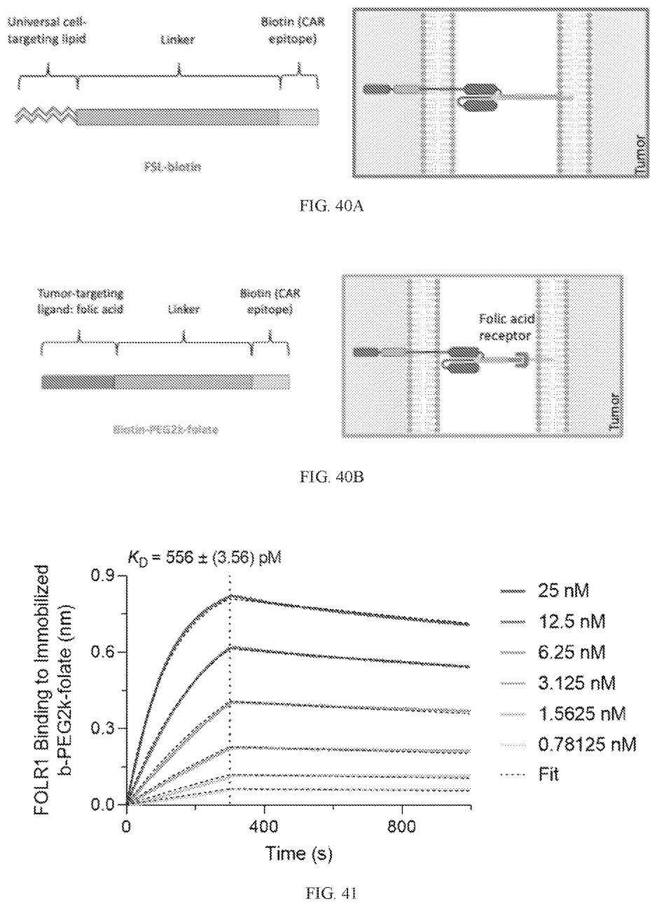

Once the cells were manufactured and antigen binding was confirmed, the cells were subjected to a rapid cell expansion protocol (REP) for in vitro functional assays (FIG. 39). To test the versatility of targeting biotin conjugates, two bifunctional intermediate adaptors were used to label different tumor lines. Two commercially available biotinylated molecules were selected. The first molecule, Function-spacer-lipid (FSL)-biotin (FSL-biotin; or “intermediate 1”), was used to label K562 cells (FIGS. 10, 40A). See e.g., Henry, S., Williams, E., et al. Sci Rep 8, 2845 (2018). A second molecule, Biotin-PEG2k-folate (“intermediate 2”), was used to label folate receptor 1+(FOLR1) MDA-MB-231 cells (FIGS. 7A, 40B). FSL-biotin dose was provided by the manufacturer, and a saturating biotin-PEG2k-folate dose was determined using biolayer interferometry (BLI) to measure biotin-PEG2k-lipid binding to immobilized FOLR1 (FIG. 41). K562 cells labeled with FSL-biotin and MDA-MB-231 cells labeled with biotin-PEG2k-folate were stained with streptavidin-APC and analyzed with flow cytometry. Resulting histograms indicated strong biotin signal from labeled cells (FIGS. 24A, 24B).

Expression of various activation markers and the makeup of cytokines produced by the CAR T cells in response to stimulation with targets was assessed. To this end, K562 P and MDA-MB-231 cells were labeled with their respective intermediate adaptors and incubated with mixed CD4/CD8 anti-biotin CAR T cells at an E:T ratio of 1:1 for 4 hours. Cells were then harvested, and activation marker (CD107a, nur77, and 41BB) expression (FIGS. 12A, 12B, 13A, 13B, 14A, 14B) and cytokine production (IFNγ, TNFα, and IL2) (FIG. 15) was assessed using intracellular cytokine staining (ICCS). Among CD8⁺ populations, every CAR T cell had upregulated activation marker expression when stimulated with intermediate-labeled targets. For each spacer length, the VLVH scFv orientation had a higher percentage of marker⁺ cells than the VHVL orientation. These trends were conserved when looking at cytokine production among CD4⁺ populations.

Cytokines released into the environment when interacting with target cells was evaluated. K562 P and MDA-MB-231 cells were labeled with their respective intermediates and incubated with anti-conjugated biotin CAR T cells at an E:T of 1:1 for 24 hours. Supernatant was harvested, and analyzed for IFNγ, TNFα, and IL2 (FIGS. 21A, 21B, 22A, 22B, 23A, 23B). In line with the data thus far, the VLVH scFv enabled a stronger T cell response than cells with the VHVL scFv orientation at each spacer length. These results indicated that long spacer CARs were best suited for FSL-biotin-labeled targets.

### Anti-Biotin CAR T Cells Lyse Tumors In Vitro and In Vivo

Anti-tumor efficacy of the anti-biotin CAR T cells was then assessed in vitro. Cytotoxicity of the anti-biotin CAR T cells was assessed using a chromium release assay. K562 parental (K562 P), K562 OKT3, and MDA-MB-231 target cells were loaded with Cr” overnight. Half of the K562 P and MDA-MB-231 cells were then labeled with FSL-biotin and biotin-PEG2k-folate, respectively. Mixed CD4/8 anti-conjugated biotin CAR T cells were incubated with labeled or unlabeled target cells at effector:target (E:T) ratios of 30:1, 10:1, 3:1, and 1:1 for four hours before analysis. Results demonstrate specific lysis of FSL-biotin-labeled K562 cells (FIGS. 17A, 17B) and biotin-PEG2k-folate-labeled MDA-MB-231 cells (FIGS. 20A, 20B) but no lysis of non-labeled targets (FIGS. 22A, 22B, 23A, 23B). Although all CAR T cells performed similarly when targeting biotin-PEG2k-folate, the short spacer demonstrated inferior lytic ability when targeting FSL-biotin.

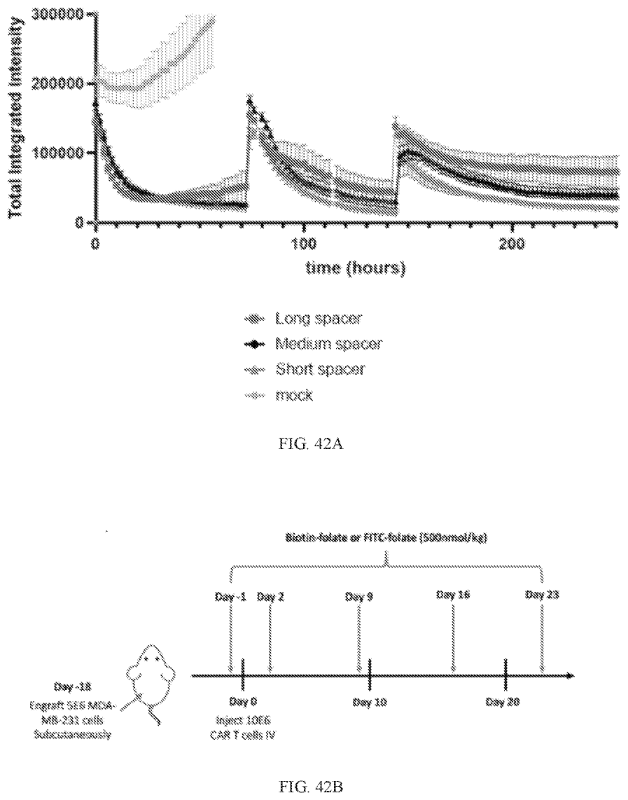

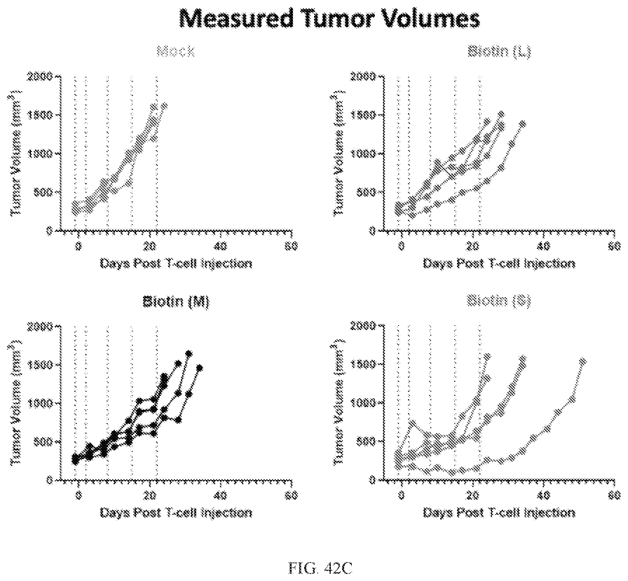

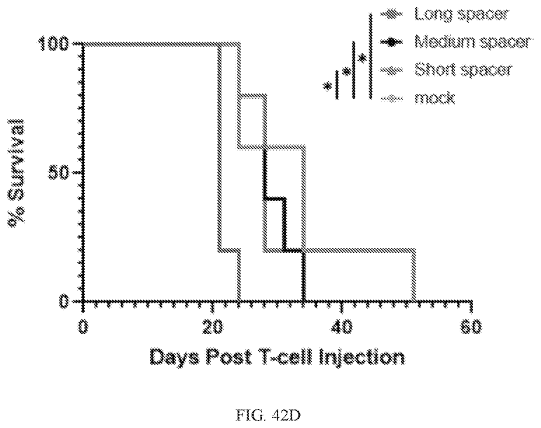

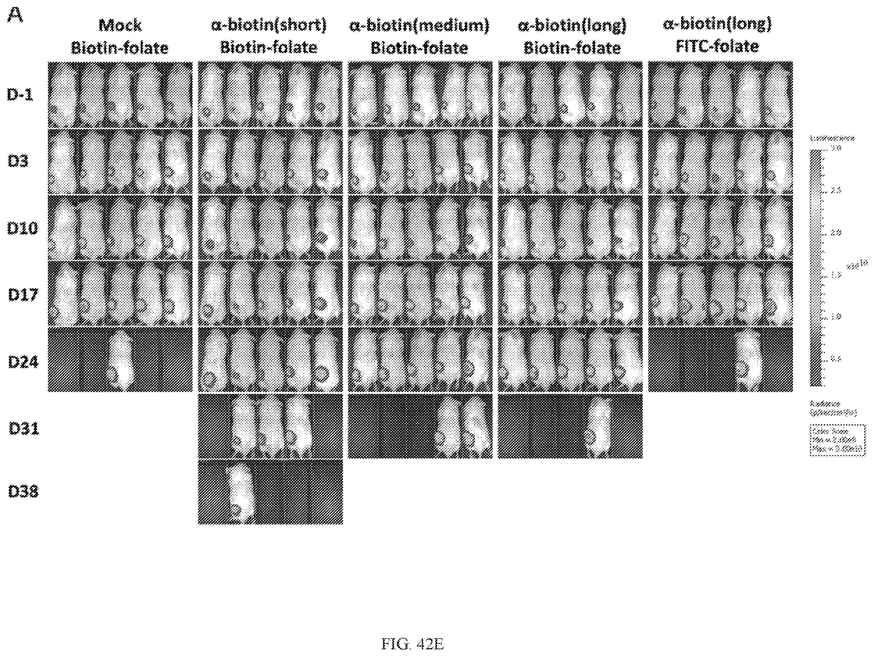

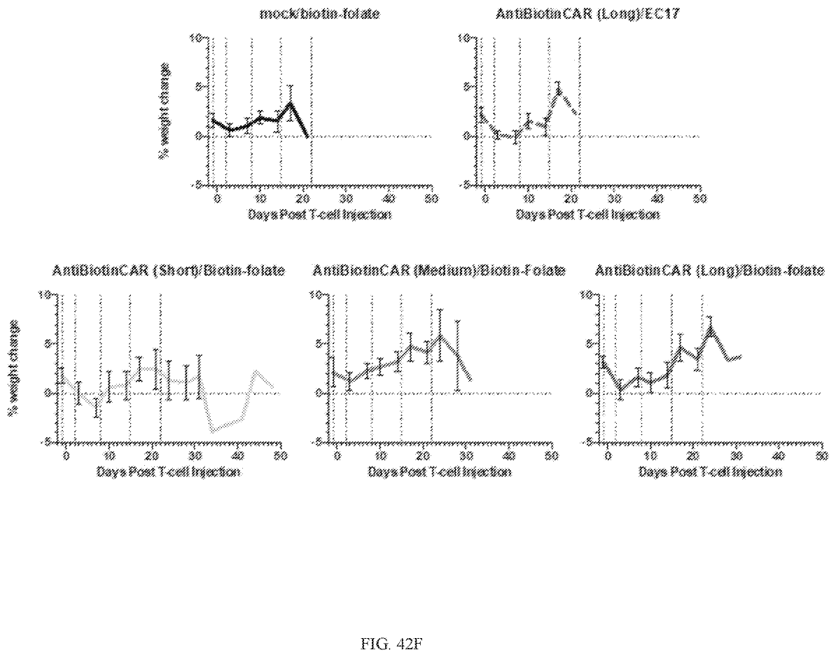

As FSL-biotin does not have relevancy for in vitro applications and VLVH orientation scFv's outperformed VHVL orientations in all assays up to this point, focus was directed toward the three VLVH CARs paired with biotin-PEG2k-folate. Mock (donor-matched, CAR-T cells) and VLVH CAR T cells were co-incubated with biotin-PEG2k-folate-labeled, mCherry MDA-MB-231 cells at a 1:1 E:T ratio and imaged every 2 hours in an IncuCyte cell imaging machine. Additional target cells were added every 72 hours, and wells were imaged for a total of 250 hours. The wells were then analyzed and the total integrated intensity (RCU×μm²/image) of mCherry signal, an indicator of the target cell population, was calculated. Results showed equivalent control of tumor growth with each CAR construct but not mock cells (FIG. 42A).

In vivo efficacy of the constructs was then assessed in a xenograft model featuring the aggressive breast cancer line MDA-MB-231.4 To enable serial bioluminescence imaging, MDA-MB-231 cells were genetically modified to express an mCherry:ffluc fusion protein. MDA-MB-231 mCherry:ffluc cells were injected subcutaneously on the flanks of NSG mice and allowed to grow to ˜200 mm³. On day −1, 500 nmol/kg of biotin-PEG2k-folate or FITC-folate (EC17) (placebo drug) was injected intravenously (IV) in accordance with previous literature.¹⁹ On day 0, 10E6 anti-biotin CAR T cells or corresponding mock cells were injected IV into mice in their appropriate groups (n=5). On day 2, another dose of 500 nmol/kg biotin-PEG2k-folate or EC17 was administered IV. Three more intermediate doses were given, spaced out every 7 days, for a total of 5 treatments (FIG. 42B). While all tumors continued to grow throughout the duration of this experiment, bioluminescence imaging (FIG. 42E) and individual tumor measurements (FIG. 42C) showed that all anti-biotin CAR T cells were able to slow the growth of tumors when compared with mock-treated mice. All CARs were able to provide a significant survival advantage compared with biotin-folate/mock-treated mice (short spacer: p=0.009, medium spacer: p=0.009, long spacer: p=0.0047) (FIG. 42D). None of the CAR T cells induced notable weight loss or other severe signs of toxicity (FIG. 42F).

### Modulating Intermediate Size and Linker Chemistry Improve Tumor Labeling and T Cell Reactivity

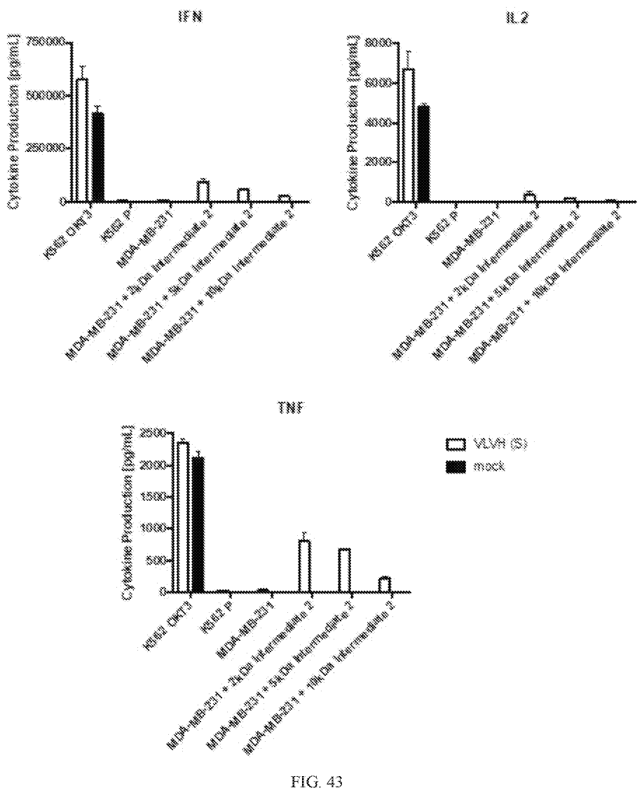

The size of biotin-folate was modulated to enhance the response of our CARs. Two larger biotin-PEG-folate conjugates (molecular weights 5 kDa and 10 kDa) were used to label MDA-MB-231 cells. Labeled targets were then incubated for 24 hours with our CAR T cells, supernatants were harvested, and cytokines were quantified with a Meso Scale Delivery (MSD) kit. Results with the short spacer CAR indicated that cytokine production decreased as the intermediate adaptor size increased (FIG. 43). Since the smallest intermediate elicited the strongest response, a 700 Dalton biotin-folate (biotin-folate 700) was designed and synthesized. In addition to producing a smaller bridge between target and T cell, this new molecule was less likely to be immunogenic than biotin-PEG2k-folate since it lacked the PEG linker.

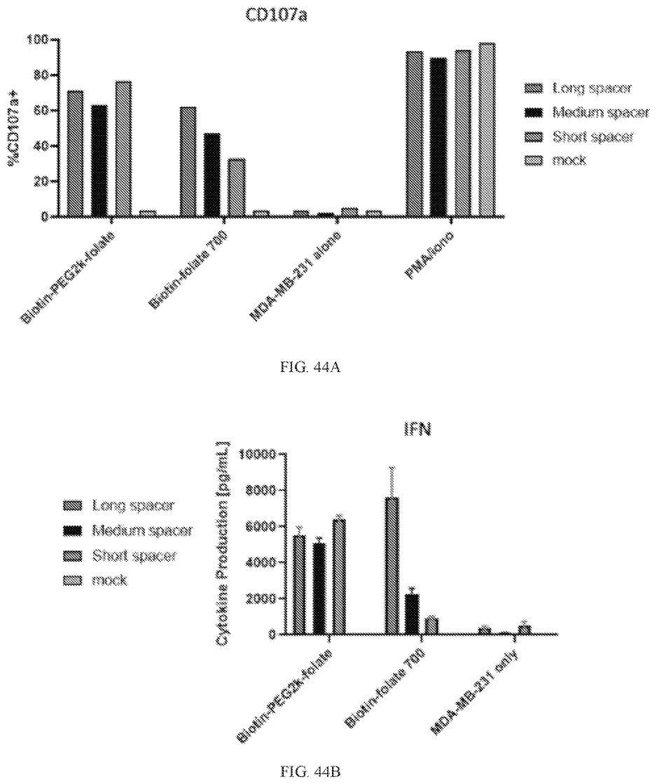

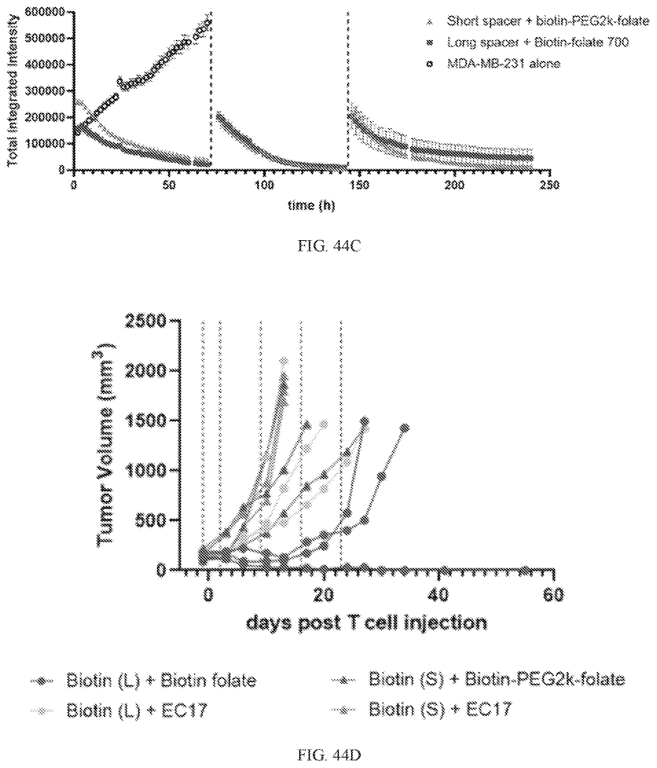

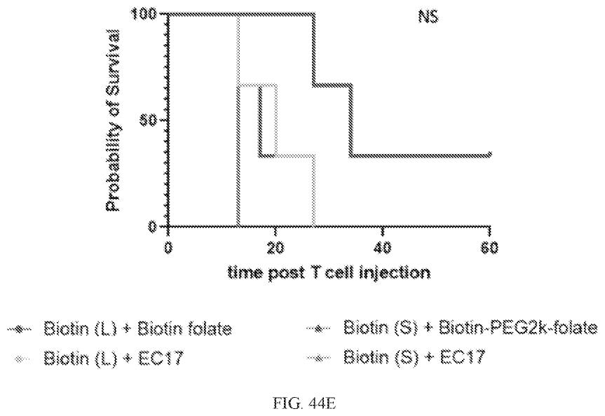

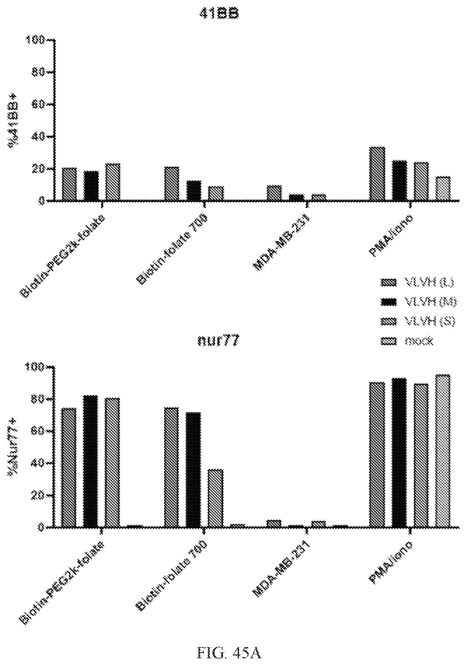

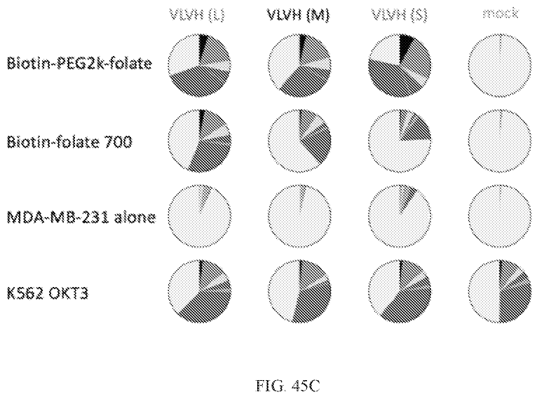

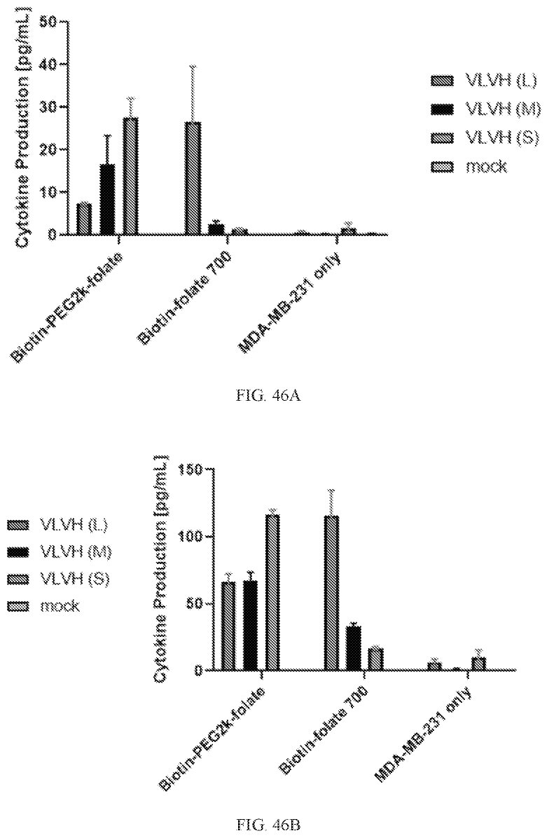

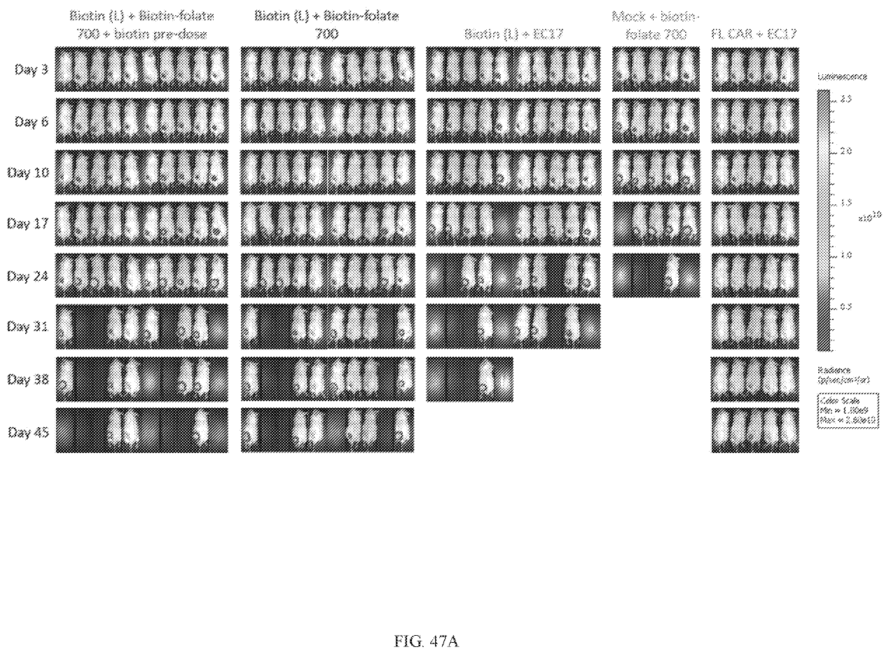

To compare the new intermediate adaptor with the original molecule, biotin-folate 700 and biotin-PEG2k-folate were used to label MDA-MB-231 cells, which were then stained with an anti-biotin secondary antibody. Flow cytometry analysis indicated enhanced tumor labeling with biotin-folate 700 compared with biotin-PEG2k-folate. The two molecules were then compared with a series of functional assays. First, MDA-MB231 cells were labeled with either biotin-folate 700 or biotin-PEG2k-folate and co-incubated with VLVH anti-biotin CAR T cells at a 1:1 E:T ratio for 4 hours. The cells were harvested and underwent intracellular cytokine staining (ICCS) of activation markers CD107a, nur77, and 41BB (FIG. 44A, FIG. 45A) and cytokines IFNγ, TNFα, and IL2 (FIG. 45B). Labeled targets were then incubated with anti-biotin CAR T cells at an E:T of 1:1 for 24 hours. At the end of the incubation, supernatant was harvested, and analyzed for IFNγ, TNFα, and IL2 concentrations (FIG. 44B, FIGS. 46A, 46B). Collectively, these data indicated that the long spacer CAR paired the best with biotin-folate 700, resulting in similar outputs to MDA-MB-231 cells labeled with biotin-PEG2k-folate. To assess anti-tumor efficacy, biotin-folate 700-labeled MDA-MB-231 cells and long spacer anti-biotin CAR T cells were co-incubated in parallel with biotin-PEG2k-folate-labeled MDA-MB-231 cells and short spacer anti-biotin CAR T cells at a 1:1 E:T ratio in an IncuCyte cell imaging system. As performed before, cells were imaged every 2 hours and targets were re-dosed every 72 hours. Results indicated similarly controlled tumor populations in both groups (FIG. 44C).

The pairings of the short spacer CAR+ biotin-PEG2k-folate and the long spacer CAR+ biotin-folate 700 were then compared head-to-head in a small xenograft model using the same experimental design previously described (FIG. 42B). Biotin receptors are present throughout the body, and MDA-MB-231 cells have moderate biotin receptor expression. To account for the possibility that biotin receptors were sequestering the intermediates from therapeutic relevancy, all mice were also pre-dosed IV with 5 mg/kg unmodified biotin 10 minutes before injections to saturate these receptors. Compared with their placebo groups, trends suggested that CARs were able to slow tumor growth when provided their respective intermediates (FIG. 44D). The tumor in one mouse treated with the long spacer+biotin-folate 700 was fully regressed. The “short spacer+biotin-PEG2k-folate” group had similar activity to the “long spacer+biotin-folate 700 group”; however, the latter group produced a disease-free mouse in an initial study.

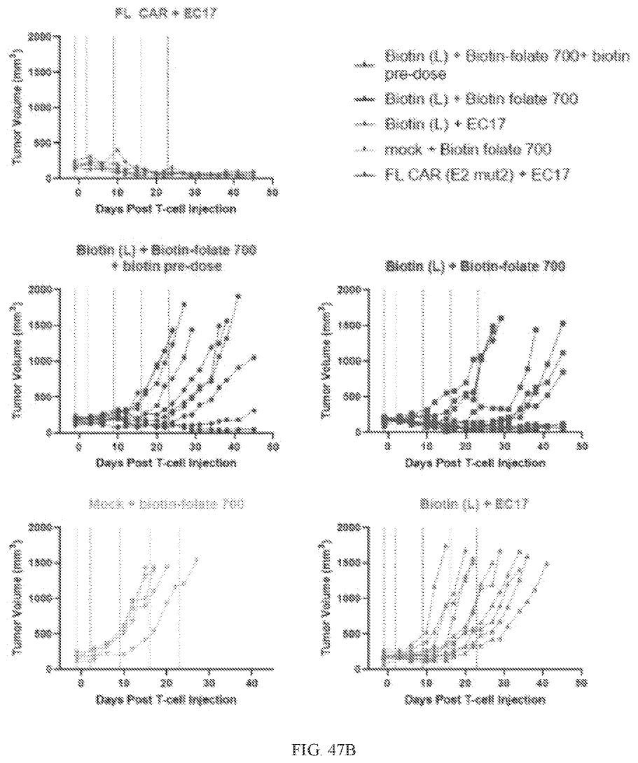

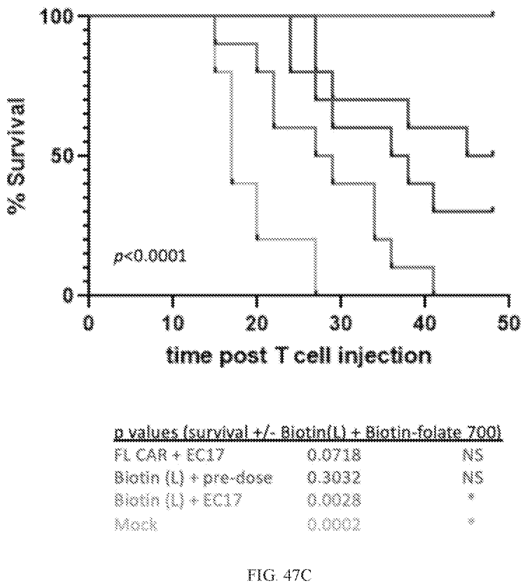

A large-scale in vivo experiment was performed to interrogate the reactivity of cells in biotin-folate 700-treated mice further. To answer whether the biotin pre-dose is necessary, a pre-dose group and a group without the pre-dose were included. Also, one group was treated with an anti-FL CAR and EC17 to serve as a positive control. The long spacer CAR+biotin-folate 700 treatment group provided significant therapeutic benefit to the animals. IVIS images showed tumor regression in 7/10 mice in this group with 2/10 potentially being rendered disease-free (FIG. 47A). In the 7/10 mice responsive to the therapy, tumor growth was tightly controlled until completion of drug dosing on day 23 post-T cell injection (FIG. 47B), thereby illustrating the dependency of CAR T cell function on the intermediate. In addition, the long spacer CAR+biotin-folate 700 provided a significant survival benefit to treated mice over both negative control groups (p=0.0028 compared with long spacer+EC17 and p=0.0002 compared with mock+biotin folate 700) (FIG. 47C). There was no statistical difference between the long spacer+biotin-folate 700 and anti-FL CAR+EC17 (p=0.0718). Pre-dosing biotin before biotin-folate 700 did not improve the efficacy of the therapy by any metric.

### Discussion

Universal enable targeting multiple antigens with a single mono-specific CAR, thereby providing a new way to target a multitude of different tumor antigens with a single CAR T cell product. Since the receptor epitope on these molecules is generally non-native, universal CAR T cells are orthogonal to the body, and their function is tied to the dosing and pharmacokinetic properties of the intermediate adaptors. This feature provides a level of control more reflective of small molecule and protein therapies, therefore imparting several benefits unique to these systems. For instance, CAR T cell persistence can be promoted with periodic, rather than continuous, delivery of antigen to minimize T cell overstimulation. This feature also makes universal CAR systems uniquely capable of mitigating toxicities due to the ability to cease or block of antigen presentation to quench overreactive T cells.

However, these systems are often highly synthetic in nature, and there are immunogenicity concerns surrounding many of them. Non-human-derived molecules on intermediate adaptors, such as PNE and FITC, may elicit a host immune response.^{25,45} On the other hand, human derived PNE may induce autoimmunity issues.²¹ One target, biotin, or vitamin B7, appears to circumvent immunogenicity concerns with the intermediate adaptors. However, the first anti-biotin receptors are avidin- and streptavidin-based, meaning they bind all versions of biotin, including dietary biotin in circulation.^{28,29} This likely has a quenching effect on CAR T cell function, thereby limiting their in vivo applications. Furthermore, avidin and streptavidin are non-human derived and known to be immunogenic.^{46,47} To reduce the immunogenicity of the CAR while targeting a biologically compatible molecule, like biotin, another CAR that binds a specific biotin-linker conjugate was previously developed.³¹ This receptor, adCAR, demonstrated selective binding to their biotin conjugate over environmental biotin, as well as robust in vitro and in vivo efficacy. However, the adCAR may be limited by its required linker sequence since it mandates the use of relatively larger intermediate molecules, which as described herein was detrimental with some targets (FIG. 43). The adCAR scFv was also murine-derived, which has been shown to produce lower proliferation, lower cytotoxicity, shorter survival, and a stronger host response to the CAR than human-derived scFv's.⁴⁸⁻⁵⁰

As described herein, a panel of fully humanized CARs that specifically bind conjugated derivatives of biotin, irrespective of linker chemistry. This system incorporated the benefits of targeting a small, endogenous molecule like biotin while maximizing biocompatibility and flexibility in intermediate adaptor design. The panel of CAR T cells was matched with different tumors labeled with different biotinylated intermediate adaptors and linker chemistries. Despite observing high efficacy in vitro, the original in vivo model described herein was not as effective as would have been expected. As a result, the modularity of the system was used, and a smaller intermediate adaptor was developed to better match with one of the CARs described herein. Pairing this new molecule with the VLVH long spacer CAR, restricted tumor growth during the intermediate adaptor treatment window was observed. This ultimately resulted in a significant survival benefit and complete and durable regression of aggressive MDA-MB-231 tumors in 20% of mice.

Because of its biocompatibility and versatility, the anti-biotin CARs described herein can be integrated with other CAR T cell technologies to create a universal CAR T cell therapy. For example, the CARs can be combined with allogenic T cells to provide an “off-the-shelf product” that can target any variety of antigens. To this end, the CAR T cells can be paired with other adaptor molecules for combinatorial or sequential targeting of different antigens.

### Materials and Methods

### Cloning of Lentiviral Constructs

All oligonucleotides were synthesized by Integrated DNA Technologies. DNA fragments encoding a Kozac sequence, open reading frame encoding a GM-CSF signal, and our anti-biotin CARs (scFv, spacer, transmembrane domain, signaling domains, DHFRdm, and EGFRt) were codon optimized for humans using ThermoFisher Scientific's GeneArt Gene Optimizer and synthesized by ThermoFisher Scientific's GeneArt Gene Synthesis. Products were amplified by PCR using the Phusion High-fidelity DNA polymerase (NEB) prior to cloning. The six anti-biotin CARs, VHVL and VLVH scFv orientations with short (IgG4 hinge), medium (IgG4 hinge-CH3), and long (IgG4 hinge-CH2-CH3) extracellular spacers each, were cloned into epHIV7.3 lentiviral vectors. To do this, the lentiviral vector was digested with NheI and NotI restriction enzymes (NEB) and gel purified with a Zymoclean Gel DNA Recovery kit (Zymo Research). Anti-biotin CAR DNA fragments were then cloned into the lentiviral vectors via Gibson assembly using NEBuilder® HiFi DNA Assembly master mix (NEB). Stellar™ chemically competent E. coli(Takara) were transformed with the Gibson assembly products, and kanamycin-selected colonies were picked for DNA isolation via minipreps (Qiagen). Ends of inserts were then sequenced by Sangar sequencing (Genewiz). Final colonies were then selected and grown up for transfection-grade plasmid DNA preparation with maxipreps (Machery-Nagel). Full CAR sequencing was conducted to ensure DNA quality using Sanger Sequencing (Genewiz).

### Biotin-Folate 700 Synthesis

A Liberty Blue synthesizer (CEM corp.) for standard solid-phase peptide synthesis (SPPS) methods on a Biotin Novatag resin (Novabiochem) was used. Briefly, FMOC-protected gGlu was mixed (4-fold molar excess) in dimethylformamide (DMF; Fisher Scientific) with dicyclohexylcarbodiimide (DIC; Chem-Impex Intl.) and ethyl cyanohydroxyiminoacetate (Oxyma; Chem-Impex Intl.) and incubated at 98° C. for 4 minutes. The resin was rinsed with DMF, and the Fmoc-protecting group was removed in 20% piperidine (Sigma Aldrich) to expose the amino-terminal primary amine. N10-(trifluoroacetyl)pteroic acid (BOC Sciences) was linked to the resin with 2 molar equivalents Oxyma, and 3 molar equivalents N,N-diisopropylethylamine (DIPEA; Sigma Aldrich, Inc.) in DMF for synthesis of folate at the N-terminal position. Biotin-folate 700 was then cleaved off the resin with 5% (1,3-dimethylabenzen (DMB; Sigma-Aldrich, Inc), 2.5% triisoproplysilane (TIPS; Sigma-Aldrich, Inc) in trifluoroacetic acid (TFA; Sigma-Aldrich, Inc) and precipitated in ice cold ethyl Ether (Thermo-Fisher, Inc.) and dried overnight before purification by HPLC.

### Bio-laver Interferometry

Intermediate binding affinity to FOLR1 was determined with an Octet BLI biosensor (Sartoris). 25 nM intermediates were loaded onto streptavidin sensors (Sartoris). Titrated concentrations of FOLR1 protein (Acro Biosystems) were then introduced, and protein association/dissociation were measured. Best fit lines were made and Kd values were calculated.

### Lentivirus Production and Tittering

Lentivirus was produced by co-transfecting HIV transfer plasmid and vectors encoding packaging proteins (Rev, Gag, Pol, VSVG) in HEK293T cells using PEIpro (PolyPlus). PEIpro/DNA mixture was incubated with HEK293T cells for 24 hours in a shaking incubator. After the first incubation, 1 uL sodium butyrate/mL of media (Sigma) was added for 48 hours. After this incubation (72 hours after transfection), viral cultures were harvested and centrifuged at 1000×g for 15 minutes. The supernatants were then harvested and filtered through a 45 um Sartopure PP3 filter (Sartorius Stedim). Filtered supernatants were then centrifuged at 10,000×g for 4 hours at 4° C. After the spin, the supernatant was removed, and virus pellets were resuspended in Opti-MEM reduced serum medium (ThermoFisher Scientific). The viruses were then stored at −80° C. until further use.

To titer our viruses, 1E5 H9 cells were transduced with 0, 0.05, 0.1, 0.25, 0.5, 1, and 3 μL of virus in 500 uL RPMI (Gibco) with a final concentration of 10% heat-inactivated and gamma irradiated FBS (VWR) and 2 mmol/L L-Glutamine (ThermoFisher Scientific) (Complete RPMI) with 40 ug/mL protamine sulfate(Fresenius Kabi). The next morning, 1 mL of complete RPMI was added to the cells to dilute the protamine sulfate. 72 hours later, cells were stained with Erbitux-APC (BD Biosciences) to measure the percentage of EGFRt+ cells. Titers were calculated from virus dilutions that produced positive cell populations in the linear tittering range (roughly 15-45%) using the following equation.

$$\frac{TU}{mL} = \frac{( \% EGFRt + ) ( 10 E 5 cells )}{mL of lentivirus} .$$

### T Cell Isolation and Characterization

For both donors described herein, Leukopak leukapheresis products were obtained from STEMCELL Technologies. Leukopak contents were drained and centrifuged at 500×g for 3 hours and 30 minutes at room temperature. Supernatants were then aspirated, and cell pellets were resuspended. Magnetic CD8+ sorting beads (Miltenyi) were added to the cells and incubated on a nutating rocker (ThermoFisher Scientific) at 4° C. for 30 minutes. The magnetically labeled cells were then added to sorting columns in a MultiMACS24 (Miltenyi). After draining, the columns were washed and eluted. Cells were counted, an aliquot was taken for FACS analysis, and the rest were frozen in CyroStor CS5 (StemCell) for future use. To get CD4+ T cells, the negative fraction from the CD8+ T cell sort was removed from the MultiMACS24 and mixed with magnetic CD4+ sorting beads (Miltenyi). The same process used with CD8+ cells was used to sort CD4+ cells. Once cells were counted, an aliquot was taken for FACS analysis, and the rest of the cells were frozen for future use. The cell aliquots set aside during isolations were then stained with anti-CD45RO BUV395 (DB Biosciences), anti-CD62L PE (BioLegend), anti-CD3 BV421 (BD Biosciences), anti-CD4 BV785 (BioLegend), and anti-CD8 APC (BioLegend). After the stain and subsequent washes, cells were fixed in 0.5% paraformaldehyde and kept in the dark at 4° C. until running on a LSRII Fortessa (BD Biosciences). Compensation was performed using tubes of UltraComp eBeads Compensation Beads (ThermoFisher Scientific) individually stained for each specific fluorophore used, and the compensation matrix was calculated using FlowJo Software (TreeStar). FlowJo software was also used for data analysis.

### T Cell Transduction and Expansion

Pure CD4 and CD8 thawed, washed, and resuspended at a 1:1 ratio at 2.5E6 cells/mL in X-VIVO-15 medium (Lonza) supplemented with 2% KnockOut serum replacement (Gibco), 4.6 ng/mL IL2 (STEMCELL Technologies), 5 ng/mL IL7 (Miltenyi), 0.5 ng/mL IL15 (Miltenyi), and 1 ng/uL IL21 (Miltenyi). 10E6 mixed CD4/8 cells in 4 mL were stimulated with CD3/CD28 CTS Dynabeadsrm (ThermoFisher Scientific) overnight in a 12-well plate (Day −1). The next day, 3 mL were removed from each well, and 40 μg/mL protamine sulfate (Fresenius Kabi) was added to each well. Viruses were thawed, vortexed, and added to each corresponding well in volumes to enable transduction at an MOI of 1 (Day 0). Plates were returned to an incubator for 24 hours. The next day, the 3 mL of media removed the day prior was added back to each well (Day 1). On Day 3 or 4, cultures were moved up to 6-well G-REX (Wilson Wolf Manufacturing Company) and cytokines were re-supplemented. 50 nM methotrexate (Medline) was also added to appropriate wells to select CAR+ T cells. A half media change with cytokine and methotrexate re-supplementation was also performed on day 7. On day 9, stimulation beads were removed, and an aliquot of cells were removed for characterization via flow cytometry. Characterization included CD4/8 splits, EGFRt expression, and a direct CAR stain using anti-CD4 FITC(BioLegend), anti-CD8 PE (Biolegend), erbitux-APC (BD Biosciences), and AF647-F(ab′)2 (Jackson ImmunoResearch) antibodies. Compensation was performed using UltraComp eBeads Compensation Beads and analyzed using FlowJo Software (TreeStar).

The remaining cells were either frozen for use in in vivo experiments or expanded with a rapid expansion protocol. Here, transduced T cells were expanded by stimulation with irradiated (8000 rad) TM-LCL and PBMC cells. Cells were cultured in Complete RPMI, 30 ng/mL OKT3 (Miltenyi) and cytokine concentrations listed above. Functional assays were performed starting on day 11 of this expansion protocol.

### Antigen Binding and Competition Assay

To confirm anti-biotin CAR T cell binding to conjugated biotin, 440 nM of a 1.1 kDa biotin-PEG-Cy5 (Click Chemistry Tools) conjugate was incubated with the cells for 30 min at room temperature. For the competition assay, 0.2 nM, 1 nM, 5 nM, or 20 nM of either 2 kDa biotin-folate (NanoCS) or unconjugated biotin (Sigma-Adrich) were incubated concurrently with the cells and biotin-PEG-Cy5. Cells were washed and fixed in a 0.5% paraformaldehyde solution. They were then kept in the dark at 4° C. until running on a LSRII Fortessa (BD Biosciences). FlowJo software was used for data analysis.

### Tumor Line Culturing

The erythroleukemia K562 cell line was obtained from the European Collection of Cell Cultures through Sigma-Aldrich. K562 cells transduced to express an anti-CD3 agonist OKT3scFv were made by lentiviral transduction of an OKT3scFv-CD4tm-T2A-Her2tG_epHIV7.2 vector into the K562 parental cell line. K562 cell lines were cultured in complete RPMI in 5% CO₂ at 37° C. MDA-MB-231 cells were cultured in low-glucose, folic acid-depleted DMEM (Sigma-Aldrich) with replenished glucose and supplemented with HEPES (Gibco), 10% heat-inactivated and gamma irradiated FBS (VWR), 2 mmol/L L-Glutamine (ThermoFisher Scientific), and 3.7 g/L sodium bicarbonate (ThermoFisher Scientific). All lines were tested for mycoplasma contamination.

Labeling Cell Lines with Intermediate Adaptors

For numerous assays, labeling targets with intermediate adaptors is necessary to stimulate the anti-biotin CAR T cells. In these studies, K562 P cells were labeled with FSL-biotin (KODE Biotech) at 10 μg/mL for 2 hours at 37° C. This intermediate was termed “intermediate 1.” MDA-MB-231 cells were labeled with either a 2 kDa, 5 kDa, or 10 kDa biotin-folate (NanoCS) at 1 uM for 30 minutes at room temperature. This intermediate was termed “intermediate 2.”

### Chromium Release Assay

Target cells were loaded with Cr⁵¹ (Perkin Elmer) overnight in 5% CO₂ at 37° C. The next day, the cells were washed and labeled with the appropriate intermediate adaptor. Some target cells were set aside to confirm labeling via flow cytometry. After another wash, labeled and unlabeled target cells were co-incubated in triplicate with T cells at various effector to target cell ratios (30:1, 10:1, 3:1 and 1:1). Supernatants were collected following the 4-hour incubation period for f-counting using Top Count NTX (Perkin Elmer). Specific lysis was calculated as previously described.⁵⁷

### Intracellular Cytokine Stain (ICCS) Assay

Cytokine expression and activation/exhaustion markers of anti-biotin CAR T cells stimulated with targets cells were examined by ICCS. Target cells were labeled with appropriate intermediate adaptors and washed. T cells were then co-incubated with labeled and unlabeled target cells at an E:T ratio of 1:1 for 4 hours in the presence of Protein Transport Inhibitor (ThermoFisher Scientific) to block cytokine secretion. Cells were incubated in the appropriate medium for each target line. 100× Cell Stimulation Cocktail (ThermoFisher Scientific) was used as a positive control. An anti-CD107a BV711 antibody (BioLegend) was also included with the cells during the incubation. Cells were then labeled for dead cells using FVS520 live/dead stain (BD Horizon), washed, and subsequently FcR Blocked (Miltenyi Biotec). Surface markers were then stained using anti-CD4 PacBlue(BioLegend), anti-CD8 PerCP/Cy5.5 (Biolegend), and erbitux-APC (BD Biosciences). Following surfacing staining, cells were fixed and permeabilized according to manufacturer instructions using BD Cytofix/Cytoperm Fixation/Permeabilization Solution Kit (BD Biosciences). Intracellular cytokine staining was then performed using anti-TNFα BUV395, anti-IFNγ BV786 (BD Biosciences), anti-IL-2 PE-Cy7, anti-Nur77 PE, and anti-CD137 (4-1BB) APC-fire (Biolegend). Cells were resuspended in PBS and kept in the dark at 4° C. until running on a LSRII Fortessa (BD Biosciences). Compensation was performed using UltraComp eBeads Compensation Beads (ThermoFisher Scientific) and FlowJo Software (TreeStar) to analyze data.

### Cytokine Release Assay Using a Meso Scale Delivery (MSD) Kit

Cytokine released into supernatant by anti-biotin CAR T cells stimulated with target cells for 24 hours was quantified with an MSD kit. To do this, target cells were labeled with appropriate intermediate adaptors and washed. T cells were then co-incubated with labeled and unlabeled target cells at an E:T ratio of 2:1 for 24 hours. After the incubation, supernatants were harvested and frozen for future analysis with an MSD kit. For the analysis, calibrator standards were made using provided reagents. Frozen supernatants were thawed, and an aliquot was diluted 1:50 or 1:40. Standards and diluted supernatants were added to rinsed MSD plates. Detection antibodies for IFNγ, TNFα, and IL2 were added to each well and incubated for 2 hours. Plates were then washed and run on a QuickPlex SQ 120 MSD instrument. Data was analyzed using the MSD DISCOVERY WORKBENCH immunoassay analysis software.

### In Vivo Experiments

MDA-MB-231 cells were modified for bioluminescence imaging (BLI) with the addition of transgenic mCherry:ffluc. Five million MDA-MB-231 mCherry:ffluc cells were injected subcutaneously on the left flank of 11-week-old male NOD/Scrid IL-2RC^{null}(NSG) mice (Jackson Laboratory). When tumors reached the targeted volume (200 mm³ in FIGS. 3 and 4 or 150 mm³ in FIG. 5), 500 nmol/kg of 2 kDa biotin-folate (Nanocs) or FITC-folate (EC17) was injected intravenously (IV) in accordance with previous literature.” The next day, 10E6 anti-biotin CAR T cells or corresponding mock cells were injected IV. Two days after T cell injection, another dose of 500 nmol/kg biotin-folate or EC17 was administered IV. Three more intermediate doses were given, spaced out every 7 days, for a total of 5 treatments (FIG. 3D). Starting the week before T cell injection and continuing twice per week until the completion of the study, tumor measurements and bioluminescence imaging were performed. For imaging, mice were anesthetized by isoflurane and imaged 15 minutes post-intraperitoneal (IP) injection of 4.29 mg/mouse D-luciferin (Xenogen) using the IVIS Spectrum Imaging system (Perkin Elmer). Luciferase activity (photon flux) was analyzed using Living Image Software (Perkin Elmer). All experiments were approved by the Institutional Animal Care and Use Committee.

### Statistics

For survival statistics on Kaplan-Meier Curves, a Mantel-Cox log-rank test was performed with a Bonferroni correction for multiple comparisons. “*” denotes significance of p<0.0167 for three comparisons and p<0.0125 for four comparisons (FIG. 5C).

### REFERENCES

1. Maude, S. L. et al. Chimeric antigen receptor T cells for sustained remissions in leukemia. N. Engl. J. Med. 371, 1507-1517 (2014).
2. Porter, D. L., Levine, B. L., Kalos, M., Bagg, A. & June, C. H. Chimeric antigen receptor-modified T cells in chronic lymphoid leukemia. N. Engl. J. Med. 365, 725-733 (2011).
3. Brentjens, R. J. et al. CD19-targeted T cells rapidly induce molecular remissions in adults with chemotherapy-refractory acute lymphoblastic leukemia. Sci. Transl. Med. 5, 177ra38 (2013).
4. Brentjens, R J. et al. Safety and persistence of adoptively transferred autologous CD19-targeted T cells in patients with relapsed or chemotherapy refractory B-cell leukemias. Blood 118, 4817-4828 (2011).
5. Lee, D. W. et al. T cells expressing CD19 chimeric antigen receptors for acute lymphoblastic leukaemia in children and young adults: a phase 1 dose-escalation trial. Lancet 385, 517-528 (2015).
6. Xu, X. et al. Mechanisms of relapse after CD19 CAR T-cell therapy for acute lymphoblastic leukemia and its prevention and treatment strategies. Front. Immunol. 10, 2664 (2019).
7. Orlando, E. J. et al. Genetic mechanisms of target antigen loss in CAR19 therapy of acute lymphoblastic leukemia. Nat. Med. 24, 1504-1506 (2018).
8. Sotillo, E. et al. Convergence of acquired mutations and alternative splicing of CD19 enables resistance to CART-19 immunotherapy. Cancer Discov. 5, 1282-1295 (2015).
9. Gardner, R. et al. Acquisition of a CD19-negative myeloid phenotype allows immune escape of MLL-rearranged B-ALL from CD19 CAR-T-cell therapy. Blood 127, 2406-2410(2016).
10. Wang, Z., Wu, Z., Liu, Y. & Han, W. New development in CAR-T cell therapy. J. Hematol. Oncol. 10, (2017).
11. Martinez, M. & Moon, E. K. CAR T cells for solid tumors: New strategies for finding, infiltrating, and surviving in the tumor microenvironment. Front. Immunol. 10, 128 (2019).
12. Bagley, S. J. & O'Rourke, D. M. Clinical investigation of CAR T cells for solid tumors: Lessons learned and future directions. Pharmacol. Ther. 205, 107419 (2020).
13. Bonifant, C. L., Jackson, H. J., Brentjens, R J. & Curran, K. J. Toxicity and management in CAR T-cell therapy. Mol. Ther. Oncolytics 3, 16011 (2016).
14. Neill, L., Rees, J. & Roddie, C. Neurotoxicity-CAR T-cell therapy: what the neurologist needs to know. Pract. Neurol. 20, 285-293 (2020).
15. Morgan, R. A. et al. Case report of a serious adverse event following the administration of T cells transduced with a chimeric antigen receptor recognizing ERBB2. Mol. Ther. 18, 843-851 (2010).
16. Parkhurst, M. R. et al. T cells targeting carcinoembryonic antigen can mediate regression of metastatic colorectal cancer but induce severe transient colitis. Mol. Ther. 19, 620-626 (2011).
17. Gardner, R. et al. Decreased rates of severe CRS seen with early intervention strategies for CD19 CAR-T cell toxicity management. Blood 128, 586-586 (2016).
18. Minutolo, N. G., Hollander, E. E. & Powell, D. J., Jr. The emergence of universal immune receptor T cell therapy for cancer. Front. Oncol. 9, 176 (2019).
19. Lee, Y. G. et al. Regulation of CAR T cell-mediated cytokine release syndrome-like toxicity using low molecular weight adapters. Nat. Commun. 10, 2681 (2019).
20. Cho, J. H., Collins, J. J. & Wong, W. W. Universal chimeric antigen receptors for multiplexed and logical control of T cell responses. Cell 173, 1426-1438.e11 (2018).
21. Cartellieri, M. et al. Switching CAR T cells on and off: a novel modular platform for retargeting of T cells to AML blasts. Blood Cancer J. 6, e458-e458 (2016).
22. Koristka, S. et al. Retargeting of regulatory T cells to surface-inducible autoantigen La/SS-B. J. Autoimmun. 42, 105-116 (2013).
23. Nardi, N. et al. Circulating auto-antibodies against nuclear and non-nuclear antigens in primary Sjögren's syndrome. Clin. Rheumatol. 25, 341-346 (2006).
24. Pan, Z.-J. et al. Neo-epitopes are required for immunogenicity of the La/SS-B nuclear antigen in the context of late apoptotic cells. Clin. Exp. Immunol. 143, 237-248 (2006).
25. Rodgers, D. T. et al. Switch-mediated activation and retargeting of CAR-T cells for B-cell malignancies. Proc. Natl. Acad. Sci. U.S.A 113, E459-68 (2016).
26. Morfill, J. et al. Affinity-matured recombinant antibody fragments analyzed by single-molecule force spectroscopy. Biophys. J. 93, 3583-3590 (2007).
27. Kim, M. S. et al. Redirection of genetically engineered CAR-T cells using bifunctional small molecules. Am. Chem. Soc. 137, 2832-2835 (2015).
28. Urbanska, K. et al. A universal strategy for adoptive immunotherapy of cancer through use of a novel T-cell antigen receptor. Cancer Res. 72, 1844-1852 (2012).
29. Lohmueller, J. J., Ham, J. D., Kvorjak, M. & Finn, O. J. mSA2 affinity-enhanced biotin-binding CAR T cells for universal tumor targeting. Oncoimmunology 7, e1368604 (2017).
30. Zhao, J., Lin, Q., Song, Y. & Liu, D. Universal CARs, universal T cells, and universal CAR T cells. J. Hematol. Oncol. 11, 132 (2018).
31. Seitz, C. M. et al. Novel adapter CAR-T cell technology for precisely controllable multiplex cancer targeting. Oncoimmunology 10, 2003532 (2021).
32. Brinkmann, U., Georges, G., Grote, M., Hoffmann, E. & Roche Diagnostics. WO2014006123—ANTI-BIOTIN ANTIBODIES AND METHODS OF USE. Patentscope https://patentscope.wipo.int/search/en/detail.jsfrdocId=WO2014006123 (2014).
33. Lu, D., Jimenez, X., Witte, L. & Zhu, Z. The effect of variable domain orientation and arrangement on the antigen-binding activity of a recombinant human bispecific diabody. Biochem. Biophys. Res. Commun. 318, 507-513 (2004).
34. Kim, Y.-J. et al. Improving the productivity of single-chain Fv antibody against c-Met by rearranging the order of its variable domains. J. Microbiol. Biotechnol. 18, 1186-1190 (2008).
35. Hudecek, M. et al. The non-signaling extracellular spacer domain of CD19-specific chimeric antigen receptors is decisive for in vivo anti-tumor activity. Blood 120, 951-951 (2012).
36. Kunkele, A. et al. Functional tuning of CARs reveals signaling threshold above which CD8+ CTL antitumor potency is attenuated due to cell Fas-FasL-dependent AICD. Cancer Immunol. Res. 3, 368-379 (2015).
37. Hudecek, M. et al. Receptor affinity and extracellular domain modifications affect tumor recognition by ROR1-specific chimeric antigen receptor T cells. Clin. Cancer Res. 19, 3153-3164 (2013).
38. Burns, W. R. et al. A high molecular weight melanoma-associated antigen-specific chimeric antigen receptor redirects lymphocytes to target human melanomas. Cancer Res. 70, 3027-3033 (2010).
39. Jonnalagadda, M. et al. Efficient selection of genetically modified human T cells using methotrexate-resistant human dihydrofolate reductase. Gene Ther. 20, 853-860 (2013).
40. Lu, Y. J. et al. Preclinical evaluation of bispecific adaptor molecule controlled folate receptor CAR-T cell therapy with special focus on pediatric malignancies. Front. Oncol. 9, 151 (2019).
41. Welsh, J. Animal models for studying prevention and treatment of breast cancer. in Animal Models for the Study of Human Disease 997-1018 (Elsevier, 2013).
42. Chen, B.-M., Cheng, T.-L. & Roffler, S. R. Polyethylene glycol immunogenicity: Theoretical, clinical, and practical aspects of anti-polyethylene glycol antibodies. ACS Nano 15, 14022-14048 (2021).
43. Ren, W. X. et al. Recent development of biotin conjugation in biological imaging, sensing, and target delivery. Chem. Commun. (Camb.) 51, 10403-10418 (2015).
44. Bongarzone, S. et al. Imaging biotin trafficking in vivo with positron emission tomography. J. Med. Chem. 63, 8265-8275 (2020).
45. Ma, J. S. Y. et al. Versatile strategy for controlling the specificity and activity of engineered T cells. Proc. Natl. Acad. Sci. U.S.A. 113, E450-8 (2016).
46. Martorell, A. et al. Position document: IgE-mediated allergy to egg protein. Allergol. Immunopathol. (Madr.) 41, 320-336 (2013).
47. Jain, A., Barve, A., Zhao, Z., Jin, W. & Cheng, K. Comparison of avidin, neutravidin, and streptavidin as nanocarriers for efficient siRNA delivery. Mol. Pharm. 14, 1517-1527 (2017).
48. Turtle, C. J. et al. CD19 CAR-T cells of defined CD4+:CD8+ composition in adult B cell ALL patients. J. Clin. Invest. 126, 2123-2138 (2016).
49. Wang J. et al. Activity comparison of humanized CD19 CAR-T cells with murine CD19 CAR-T on Nalm-6 cells and xenograft tumor model. Zhonghua Zhong Liu Za Zhi 43, 827-832 (2021).
50. Khan, A. N. et al. Immunogenicity of CAR-T cell therapeutics: Evidence, mechanism and mitigation. Front. Immunol. 13, 886546 (2022).
51. Hudecek, M. et al. The nonsignaling extracellular spacer domain of chimeric antigen receptors is decisive for in vivo antitumor activity. Cancer Immunol. Res. 3, 125-135 (2015).
52. Zhang, A. Q. et al. Universal redirection of CAR T cells against solid tumours via membrane-inserted ligands for the CAR. Nat. Biomed. Eng. (2023) doi:10.1038/s41551-023-01048-8.
53. Srivastava, S. et al. Logic-gated ROR1 chimeric antigen receptor expression rescues T cell-mediated toxicity to normal tissues and enables selective tumor targeting. Cancer Cell 35, 489-503.e8 (2019).
54. Moghimi, B. et al. Preclinical assessment of the efficacy and specificity of GD2-B7H3 SynNotch CAR-T in metastatic neuroblastoma. Nat. Commun. 12, 511 (2021).
55. Choe, J. H. et al. SynNotch-CAR T cells overcome challenges of specificity, heterogeneity, and persistence in treating glioblastoma. Sci. Transl. Med. 13, (2021).
56. Wei, J. & Jensen, M. C. GENERATING MAMMALIAN T CELL ACTIVATION INDUCIBLE SYNTHETIC PROMOTERS (SYN+PRO) TO IMPROVE T CELL THERAPY. World Intellectual Property Organization https://patentscope.wipo.int/search/en/detail.jsf9docId=WO2018213332 (2023).
57. Assay for cell death: Chromium release assay of cytotoxic ability. https.//www.jove.com/v/10505/assay-for-cell-death-chromium-release-assay-of-cytotoxic-ability.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. An isolated polynucleotide encoding a chimeric antigen receptor (CAR) capable of specifically binding to biotin conjugated to a molecule (conjugated biotin), wherein the CAR comprises:
(i) an scFv domain configured to bind to conjugated biotin;
(ii) a spacer domain;
(iii) a transmembrane domain; and
(iv) an intracellular signaling domain.

2-73. (canceled)
